SEQUENCE LISTING (20 sequences)

SEQ ID NO: 1 - protein, 314 aa, Escherichia coli
MPEVQTDHPETAELSKPQLRMVDLNLLTVFDAVMQEQNITRAAHVLGMSQPAVSNAVARLKVMFNDELFVRYGRGIQPTARAFQLFGSVRQALQLVQNELPGSGFEPASSERVFHLCVCSPLDSILTSQIYNHIEQIAPNIHVMFKSSLNQNTEHQLRYQETEFVISYEDFHRPEFTSVPLFKDEMVLVASKNHPTIKGPLLKHDVYNEQHAAVSLDRFASFSQPWYDTVDKQASIAYQGMAMMSVLSVVSQTHLVAIAPRWLAEEFAESLELQVLPLPLKQNSRTCYLSWHEAAGRDKGHQWMEEQLVSICKR

SEQ ID NO: 2 - protein, 305 aa, Clostridium difficile
MMILIQSRGKMKCKELSEELEVSERQIKSYKMYLEQAGIFINSTPGIYGGYEIDKCNSISLIKLLDSEVSILDMINSQLEYNNDIYKNEFNNIVEKIKAVLNTGEKSDTYMDYFTVQAQRNCDYESEKNKCNEIIRAYTTKHKFWIEYYSLNSGNSERIVHPYGLFNYKSDTYMVAFCEKRFKFIDFKLCRIKDYKVLEEKYNVDKSFSWDEYSKNSIGIYKGEEINVVIKISHPFSTIIKEKVWVNNQQIIEYDDKSIMFKAKMRGYEEIKSWILSMGAYVEVVEPDRLRNDILSEIEKMKKIY

SEQ ID NO: 3 - DNA, 32 bp, Escherichia coli
  source 1..32: mol_type = other DNA
attccggacgaagaagctgagaaaatcaccac

SEQ ID NO: 4 - DNA, 32 bp, Escherichia coli
  source 1..32: mol_type = other DNA
tatcaacggttttggccgtatcggtcgcattg

SEQ ID NO: 5 - DNA, 32 bp, Escherichia coli
  source 1..32: mol_type = other DNA
tgcaaactctgatgatgtccagtcagtatgag

SEQ ID NO: 6 - DNA, 32 bp, Escherichia coli
  source 1..32: mol_type = other DNA
aaaatggttgaaggccgcatgaagaaattcac

SEQ ID NO: 7 - DNA, 31 bp, Escherichia coli
  source 1..31: mol_type = other DNA
aggctgaaaaatcccagcagtccagttacta

SEQ ID NO: 8 - DNA, 31 bp, Escherichia coli
  source 1..31: mol_type = other DNA
gtattattcgacggcggtgggattgcttcac

SEQ ID NO: 9 - DNA, 31 bp, Escherichia coli
  source 1..31: mol_type = other DNA
aaagctgaccaggaaaaaatgggtctggctc

SEQ ID NO: 10 - DNA, 31 bp, Escherichia coli
  source 1..31: mol_type = other DNA
gctttatgtgttactctatgcgtctgcaatc

SEQ ID NO: 11 - DNA, 29 bp, synthetic construct
  misc_feature 1..29: note = Description of Artificial Sequence: Syntheticoligonucleotide
  source 1..29: mol_type = other DNA
gttttatattaactatatggaatgtaaat

SEQ ID NO: 12 - DNA, 29 bp, synthetic construct
  misc_feature 1..29: note = Description of Artificial Sequence: Syntheticoligonucleotide
  source 1..29: mol_type = other DNA
gttttagattaactatatggaatgtaaat

SEQ ID NO: 13 - DNA, 29 bp, synthetic construct
  misc_feature 1..29: note = Description of Artificial Sequence: Syntheticoligonucleotide
  source 1..29: mol_type = other DNA
gttttagattaactatatggaatgtaagt

SEQ ID NO: 14 - DNA, 29 bp, synthetic construct
  misc_feature 1..29: note = Description of Artificial Sequence: Syntheticoligonucleotide
  source 1..29: mol_type = other DNA
gttttagattaactatatggaatgtaagt

SEQ ID NO: 15 - DNA, 29 bp, synthetic construct
  misc_feature 1..29: note = Description of Artificial Sequence: Syntheticoligonucleotide
  source 1..29: mol_type = other DNA
gttttagattaactatgtggaatgtaaat

SEQ ID NO: 16 - DNA, 29 bp, synthetic construct
  misc_feature 1..29: note = Description of Artificial Sequence: Syntheticoligonucleotide
  source 1..29: mol_type = other DNA
gttttagattaactatatggaatgtaagt

SEQ ID NO: 17 - DNA, 29 bp, synthetic construct
  misc_feature 1..29: note = Description of Artificial Sequence: Syntheticoligonucleotide
  source 1..29: mol_type = other DNA
gttttagattaactatatggaatgtaagt

SEQ ID NO: 18 - DNA, 29 bp, synthetic construct
  misc_feature 1..29: note = Description of Artificial Sequence: Syntheticoligonucleotide
  source 1..29: mol_type = other DNA
gttttagattaactatatggaatgtaagt

SEQ ID NO: 19 - DNA, 29 bp, synthetic construct
  misc_feature 1..29: note = Description of Artificial Sequence: Syntheticoligonucleotide
  source 1..29: mol_type = other DNA
gttttagattaactatatggaatgtaagt

SEQ ID NO: 20 - DNA, 32 bp, Escherichia coli
  source 1..32: mol_type = other DNA
agggtctcaccaactcgacgagtcagaatcag

(19) United States
(12) Patent Application Publication
(10) Pub. No.: US 2022/0380736 A1
(43) Pub. Date: Dec. 1, 2022

(54) METHODS AND COMPOSITIONS FOR KILLING A TARGET BACTERIUM

(71) Applicant: Locus Biosciences, Inc., Morrisville, NC (US)

(72) Inventors: Paul M. GAROFOLO, Cary, NC (US); David G. OUSTEROUT, Raleigh, NC (US); Kurt SELLE, Raleigh, NC (US); Sandi WONG, South San Francisco, CA (US); Hannah Hewitt TUSON, Durham, NC (US)

(21) Appl. No.: 17/858,885

(22) Filed: Jul. 6, 2022

Related U.S. Application Data
(63) Continuation of application No. 17/469,648, filed on Sep. 8, 2021.
Application No. 17/469,648 is a division of application No. 16/899,436, filed on Jun. 11, 2020.
Application No. 16/899,436 is a continuation of application No. PCT/US2019/030695, filed on May 3, 2019.
(60) Provisional application No. 62/818,066, filed on Mar. 13, 2019.
(60) Provisional application No. 62/743,740, filed on Oct. 10, 2018.
(60) Provisional application No. 62/667,400, filed on May 4, 2018.

(51) Int. Cl. C12N 7/00; A61K 35/768; C12N 9/22

(52) U.S. Cl. CPC C12N 7/00; A61K 35/768; C12N 9/22; C12N 2795/00021; C12N 2795/00043; C12N 2795/00032; C12N 2310/20

(57) ABSTRACT

Provided herein are methods and compositions for killing a target bacterium. Also disclosed are engineered bacteriophages.

### CROSS REFERENCE

This application is a continuation of U.S. application Ser. No. 17/469,648, filed Sep. 8, 2021, which is a divisional of Ser. No. 16/899,436, filed Jun. 11, 2020, which is a continuation of International Application No. PCT/US2019/030695, filed May 3, 2019, which claims the benefit of U.S. Provisional Application No. 62/667,400, filed May 4, 2018, U.S. Provisional Application No. 62/743,740, filed Oct. 10, 2018, and U.S. Provisional Application No. 62/818,066, filed Mar. 13, 2019, all of which are incorporated herein by reference.

### SEQUENCE LISTING

The instant application contains a Sequence Listing which has been submitted electronically in XML format and is hereby incorporated by reference in its entirety. Said XML copy, created on Jul. 6, 2022, is named 53240708302_SL.xml and is 23,152 bytes in size.

### SUMMARY

Disclosed herein, in certain embodiments, are methods for killing a target bacterium. In some embodiments, the method for killing a target bacterium comprises introducing into a target bacterium a bacteriophage comprising: a first nucleic acid encoding a spacer sequence or a crRNA transcribed therefrom, wherein the spacer sequence is complementary to a target nucleotide sequence from a target gene in the target bacterium; and a gene that is capable of inducing lysis of the target bacterium. In some embodiments, the target bacterium is killed by lytic activity of the bacteriophage or activity of a CRISPR-Cas system using the spacer sequence or the crRNA transcribed therefrom. In some embodiments, the first nucleic acid sequence is a CRISPR array further comprising at least one repeat sequence. In some embodiments, the bacteriophage further comprises a second nucleic acid encoding a transcriptional activator for the CRISPR-Cas system. In some embodiments, the gene is endogenous or exogenous. In some embodiments, the transcriptional activator is regulated by quorum sensing (QS) signals. In some embodiments, the transcriptional activator is a protein involved in sensing stress of a bacterium membrane. In some embodiments, the protein involved in sensing stress is response regulator BaeSR. In some embodiments, the transcriptional activator is a protein that stabilizes Cas. In some embodiments, the protein that stabilizes Cas is heat shock protein G (HtpG). In some embodiments, the transcriptional activator is a metabolic sensing protein. In some embodiments, the metabolic sensing protein is cAMP receptor protein (CRP). In some embodiments, the CRP is sensitive to cyclic AMP (cAMP). In some embodiments, the metabolic sensing protein is a sigma factor. In some embodiments, the sigma factor is RpoN (σ⁵⁴). In some embodiments, the transcriptional activator disrupts the activity of an inhibitory element. In some embodiments, the inhibitory element comprises heat-stable nucleoid-structuring protein (H-NS), leucine responsive regulatory protein (LRP), or CodY. In some embodiments, the inhibitory element is a transcriptional repressor. In some embodiments, the transcriptional repressor is a global transcriptional repressor. In some embodiments, the transcriptional activator comprises LeuO or a polypeptide having at least 75% sequence homology with SEQ ID NO: 1. In some embodiments, the transcriptional activator comprises CD2983 or a polypeptide having at least 75% sequence homology with SEQ ID NO: 2. In some embodiments, the CRISPR-Cas system is endogenous to the target bacterium. In some embodiments, the CRISPR-Cas system is exogenous to the target bacterium. In some embodiments, the CRISPR-Cas system is a type I CRISPR-Cas system, a type II CRISPR-Cas system, or a type III CRISPR-Cas system. In some embodiments, the CRISPR-Cas system comprises the type I CRISPR-Cas system. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence is at least a portion of an essential gene that is needed for survival of the target bacterium. In some embodiments, the essential gene is Tsf, acpP, gapA, infA, secY, csrA, trmD, ftsA, fusA, glyQ, eno, or nusG. In some embodiments, the at least one repeat sequence is operably linked to the at least one spacer sequence at either its 5′ end or its 3′ end. In some embodiments, the target bacterium is killed solely by the lytic activity of the bacteriophage. In some embodiments, the target bacterium is killed solely by the activity of the CRISPR-Cas system. In some embodiments, the target bacterium is killed by both the lytic activity of the bacteriophage and the activity of the CRISPR-Cas system in combination. In some embodiments, the target bacterium is killed by the activity of the CRISPR-Cas system independently of the lytic activity of the bacteriophage. In some embodiments, the activity of the CRISPR-Cas system supplements or enhances the lytic activity of the bacteriophage. In some embodiments, the spacer nucleotide sequence overlaps with a second spacer sequence. In some embodiments, the lytic activity of the bacteriophage and the activity of the CRISPR-Cas system are synergistic. In some embodiments, the lytic activity of the bacteriophage, the activity of the CRISPR-Cas system, or both is modulated by a concentration of the bacteriophage. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the bacteriophage does not confer any new properties onto the target bacterium beyond cellular death caused by the lytic activity of the bacteriophage and/or the activity of the CRISPR-Cas array. In some embodiments, the target bacterium is C. difficile. In some embodiments, the bacteriophage is ϕCD146 or ϕCD24-2. In some embodiments, the target bacterium is E. coli. In some embodiments, the bacteriophage is T4, T7, or T7m. In some embodiments, the first nucleic acid encoding a spacer sequence or a crRNA is inserted into a non-essential bacteriophage gene. In some embodiments, the non-essential gene is gp49, gp75, or hoc. In some embodiments, the non-essential gene is gp0.7, gp4.3, gp4.5, or gp4.7. In some embodiments, the non-essential gene is gp0.6, gp0.65, gp0.7, gp4.3, or gp4.5.

In some embodiments, disclosed herein are methods for killing a plurality of target bacteria, such as in a mixed population of bacteria comprising the target bacteria and non-target bacteria (e.g., in therapeutic and/or environmental treatment processes, such as described herein). In specific embodiments, the target bacteria are treated according to any process described herein (e.g., for killing target bacterium), and a first population of the target bacteria is killed by lytic activity of the bacteriophage and a second population of the target bacteria is killed by activity of a CRISPR-Cas system using the spacer sequence or the crRNA transcribed therefrom (e.g., wherein the non-target bacteria is not killed (e.g., killed at a lesser rate than the target bacteria, such as at 50%, the rate, less than 25% the rate, less than 10% the rate, or less than 20% killed, less than 10% killed, less than 5% killed, or the like).

Disclosed herein, in certain embodiments, are methods for modulating the activity of a CRISPR-Cas system in a target bacterium. In some embodiments, the method comprises: introducing a bacteriophage comprising a nucleic acid encoding a transcriptional activator for the CRISPR-Cas system in the target bacterium. In some embodiments, the transcriptional activator is regulated by quorum sensing (QS) signals. In some embodiments, the transcriptional activator is a protein involved in sensing stress to a bacterium membrane. In some embodiments, the protein involved in sensing stress is response regulator BaeSR. In some embodiments, the transcriptional activator is a protein that stabilizes Cas. In some embodiments, the protein that stabilizes Cas is heat shock protein G (HtpG). In some embodiments, the transcriptional activator is a metabolic sensing protein. In some embodiments, the metabolic sensing protein is cAMP receptor protein (CRP). In some embodiments, the CRP is sensitive to cyclic AMP (cAMP). In some embodiments, the metabolic sensing protein is a sigma factor. In some embodiments, the sigma factor is RpoN (σ54). In some embodiments, the transcriptional activator disrupts the activity of an inhibitory element. In some embodiments, the inhibitory element is heat-stable nucleoid-structuring protein (H-NS), leucine responsive regulatory protein (LRP), or CodY. In some embodiments, the inhibitory element is a transcriptional repressor. In some embodiments, the transcriptional repressor is a global transcriptional repressor. In some embodiments, the transcriptional activator comprises LeuO or a polypeptide having at least 75% sequence homology with SEQ ID NO: 1. In some embodiments, the transcriptional activator comprises CD2983 or a polypeptide having at least 75% sequence homology with SEQ ID NO: 2. In some embodiments, the CRISPR-Cas system is endogenous. In some embodiments, the CRISPR-Cas system is exogenous. In some embodiments, the CRISPR-Cas system is a type I CRISPR-Cas system, a type II CRISPR-Cas system, or a type III CRISPR-Cas system. In some embodiments, the CRISPR-Cas system is a type I CRISPR-Cas system. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the target bacterium is C. difficile. In some embodiments, the bacteriophage is ϕCD146 or ϕCD24-2. In some embodiments, the target bacterium is E. coli. In some embodiments, the bacteriophage is T4, T7, or T7m. In some embodiments, the nucleic acid encoding a transcriptional activator is inserted into a non-essential bacteriophage gene. In some embodiments, the non-essential gene is gp49. In some embodiments, the non-essential gene is gp75. In some embodiments, the non-essential gene is hoc. In some embodiments, the non-essential gene is gp0.7, gp4.3, gp4.5, or gp4.7. In some embodiments, the non-essential gene is gp0.6, gp0.65, gp0.7, gp4.3, or gp4.5.

Disclosed herein, in certain embodiments, are methods of killing a target bacterium. The method comprises introducing into a target bacterium a bacteriophage comprising: lytic activity, and a first nucleic acid sequence encoding an anti-CRISPR polypeptide. In specific embodiments, the anti-CRISPR polypeptide enhances the lytic activity of the bacteriophage (e.g., as determined by how fast the target bacterium is killed). In some embodiments, the anti-CRISPR polypeptide inactivates a CRISPR-Cas system. In some embodiments, the anti-CRISPR polypeptide inactivates the CRISPR-Cas system using a process comprising gene regulation interference. In some embodiments, the anti-CRISPR polypeptide inactivates the CRISPR-Cas system using a process comprising nuclease recruitment interference. In some embodiments, the CRISPR-Cas system is a type I CRISPR-Cas system, a type II CRISPR-Cas system, or a type III CRISPR-Cas system. In some embodiments, the CRISPR-Cas system is a type I CRISPR-Cas system. In some embodiments, the anti-CRISPR polypeptide binds directly or indirectly to a Cascade or Cascade-like complex. In some embodiments, the anti-CRISPR polypeptide is a truncated protein, a fusion protein, a dimer protein, or mutated protein. In some embodiments, the bacteriophage further comprises a second nucleic acid encoding a CRISPR array. In some embodiments, the CRISPR array comprises at least one repeat sequence and at least one spacer sequence that is complementary to a target nucleotide sequence from a target gene in the target bacterium. In specific embodiments, provided herein are methods of killing target bacteria (e.g., in a mixed population of bacteria comprising target bacteria and non-target bacteria). The method comprises introducing into target bacteria a bacteriophage having lytic activity and comprising a first nucleic acid sequence encoding an anti-CRISPR polypeptide. In specific embodiments, the anti-CRISPR polypeptide enhances the lytic activity of the bacteriophage (e.g., as measured by number of target bacteria killed in a given amount of time).

Disclosed herein, in certain embodiments, are bacteriophages comprising: a first nucleic acid encoding a spacer sequence or a crRNA transcribed therefrom, wherein the spacer sequence is complementary to a (e.g., target) nucleotide sequence from a (e.g., target) gene in a (e.g., target) bacterium; and a gene that is capable of inducing lysis of the (e.g., target) bacterium. In specific embodiments, the target bacterium is killed by the lytic activity of the bacteriophage or activity of a CRISPR-Cas system using the spacer sequence or the crRNA transcribed therefrom. In some embodiments, the bacteriophage further comprises a second nucleic acid encoding a transcriptional activator for the CRISPR-Cas system. In some embodiments, the transcriptional activator is regulated by quorum sensing (QS) signals. In some embodiments, the transcriptional activator is a protein involved in sensing stress of a bacterium membrane. In some embodiments, the protein is response regulator BaeSR. In some embodiments, the transcriptional activator is a protein that stabilizes Cas. In some embodiments, the protein that stabilizes Cas is heat shock protein G (HtpG). In some embodiments, the transcriptional activator is a metabolic sensing protein. In some embodiments, the metabolic sensing protein is cAMP receptor protein (CRP). In some embodiments, the CRP is sensitive to cyclic AMP (cAMP). In some embodiments, the metabolic sensing protein is a sigma factor. In some embodiments, the sigma factor is RpoN (σ⁵⁴). In some embodiments, the transcriptional activator disrupts the activity of an inhibitory element of the target bacterium. In some embodiments, the inhibitory element is heat-stable nucleoid-structuring protein (H-NS), leucine responsive regulatory protein (LRP), or CodY. In some embodiments, the inhibitory element is a transcriptional repressor. In some embodiments, the transcriptional repressor is a global transcriptional repressor. In some embodiments, the transcriptional activator comprises LeuO or a polypeptide having at least 75% sequence homology with SEQ ID NO: 1. In some embodiments, the transcriptional activator comprises CD2983 or a polypeptide having at least 75% sequence homology with SEQ ID NO: 2. In some embodiments, the CRISPR-Cas system is endogenous. In some embodiments, the CRISPR-Cas system is exogenous. In some embodiments, the CRISPR-Cas system is a type I CRISPR-Cas system, a type II CRISPR-Cas system, or a type III CRISPR-Cas system. In some embodiments, the CRISPR-Cas system is a type I CRISPR-Cas system. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence is essential. In some embodiments, the essential gene is Tsf, acpP, gapA, infA, secY, csrA, trmD, ftsA, fusA, glyQ, eno, or nusG. In some embodiments, the target nucleotide sequence is a non-essential gene. In some embodiments, the first nucleic acid sequence is a CRISPR array comprising at least one repeat sequence. In some embodiments, the at least one repeat sequence is operably linked to the spacer sequence at either its 5′ end or its 3′ end. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the temperate bacteriophage is rendered lytic by the removal, replacement, or inactivation of one or more lysogeny genes. In some embodiments, the target bacterium is C. difficile. In some embodiments, the bacteriophage is ϕCD146 or ϕCD24-2. In some embodiments, the target bacterium is E. coli. In some embodiments, the bacteriophage is T4, T7, or T7m. In some embodiments, the first nucleic acid encoding a spacer sequence or a crRNA is inserted into a non-essential gene. In some embodiments, the non-essential gene is gp49. In some embodiments, the non-essential gene is gp75. In some embodiments, the non-essential gene is hoc. In some embodiments, the non-essential gene is gp0.7, gp4.3, gp4.5, or gp4.7. In some embodiments, non-essential gene is gp0.6, gp0.65, gp0.7, gp4.3, or gp4.5. Also disclosed herein, in some embodiments, are pharmaceutical compositions comprising the bacteriophage disclosed herein, and a pharmaceutically acceptable excipient. In some embodiments, the pharmaceutical compositions is in a form of a tablet, a liquid, a syrup, an oral formulation, an intravenous formulation, an intranasal formulation, an ocular formulation, an otic formulation, a subcutaneous formulation, an inhalable respiratory formulation, a suppository, or any combination thereof. Further disclosed herein, in some embodiments, are methods of treating a disease in a subject comprising administering the bacteriophage disclosed herein to the subject. In some embodiments, the subject is a mammal. In some embodiments, the disease is a bacterial infection. In some embodiments, a bacteria causing the bacterial infection is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratiurn, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter fells, Helicobacter pylori, Clostridium bolteae and any combination thereof. In some embodiments, the bacterium is a drug resistant bacterium that is resistant to at least one antibiotic. In some embodiments, the bacterium is a multi-drug resistant bacterium that is resistant to at least one antibiotic. In some embodiments, the bacterium is Pseudomonas. In some embodiments, the bacterium is staphylococcus. In some embodiments, the bacterium is Escherichia coli. In some embodiments, the bacterium is Clostridium difficile. In some embodiments, the bacterium is methicillin resistant. In some embodiments, the bacterium is methicillin resistant Staphylococcus aureus. In some embodiments, the bacterium is multidrug resistant Pseudomonas Aeruginosa. In some embodiments, the antibiotic comprises a cephalosporin, a fluoroquinolone, a carbapenem, a colistin, an aminoglycoside, vancomycin, streptomycin, or methicillin. In some embodiments, the administering is intra-arterial, intravenous, intramuscular, oral, subcutaneous, topical, inhalation, intravesical or any combination thereof.

Disclosed herein, in certain embodiments, are bacteriophages comprising a nucleic acid encoding a transcriptional activator for a CRISPR-Cas system in a (e.g., target) bacterium. In some embodiments, the transcriptional activator is regulated by quorum sensing (QS) signals. In some embodiments, the transcriptional activator is a protein involved in sensing stress to a bacterium membrane. In some embodiments, the protein involved in sensing stress is response regulator BaeSR. In some embodiments, the transcriptional activator is a protein that stabilizes Cas. In some embodiments, the protein that stabilizes Cas is heat shock protein G (HtpG). In some embodiments, the transcriptional activator is a metabolic sensing protein. In some embodiments, the metabolic sensing protein is cAMP receptor protein (CRP). In some embodiments, the CRP is sensitive to cyclic AMP (cAMP). In some embodiments, the metabolic sensing protein is a sigma factor. In some embodiments, the sigma factor is RpoN (σ54). In some embodiments, the transcriptional activator disrupts the activity of an inhibitory element. In some embodiments, the inhibitory element is heat-stable nucleoid-structuring protein (H-NS), leucine responsive regulatory protein (LRP), or CodY. In some embodiments, the inhibitory element is a transcriptional repressor. In some embodiments, the transcriptional repressor is a global transcriptional repressor. In some embodiments, the transcriptional activator comprises LeuO or a polypeptide having at least 75% sequence homology with SEQ ID NO: 1. In some embodiments, the transcriptional activator comprises CD2983 or a polypeptide having at least 75% sequence homology with SEQ ID NO: 2. In some embodiments, the CRISPR-Cas system is endogenous. In some embodiments, the CRISPR-Cas system is exogenous. In some embodiments, the CRISPR-Cas system is a type I CRISPR-Cas system, a type II CRISPR-Cas system, or a type III CRISPR-Cas system. In some embodiments, the CRISPR-Cas system is a type I CRISPR-Cas system. In some embodiments, the bacteriophage infects multiple bacterial strains. In some embodiments, the bacteriophage is an obligate lytic bacteriophage. In some embodiments, the bacteriophage is a temperate bacteriophage that is rendered lytic. In some embodiments, the target bacterium is C. difficile. In some embodiments, the bacteriophage is ϕCD146 or ϕCD24-2. In some embodiments, the target bacterium is E. coli. In some embodiments, the bacteriophage is T4, T7, or T7m. In some embodiments, the nucleic acid encoding a transcriptional activator is inserted into a non-essential bacteriophage gene. In some embodiments, the non-essential gene is gp49. In some embodiments, the non-essential gene is gp75. In some embodiments, the non-essential gene is hoc. In some embodiments, the non-essential gene is gp0.7, gp4.3, gp4.5, or gp4.7. In some embodiments, the non-essential gene is gp0.6, gp0.65, gp0.7, gp4.3, or gp4.5. Also disclosed herein, in some embodiments, are pharmaceutical compositions comprising the bacteriophage disclosed herein, and a pharmaceutically acceptable excipient. In some embodiments, the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof. Further disclosed herein are methods of treating a disease in a subject comprising administering the bacteriophage to the subject. In some embodiments, the subject is a mammal. In some embodiments, the disease is a bacterial infection. In some embodiments, a bacteria causing the bacterial infection is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratiurn, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter fells, Helicobacter pylori, Clostridium bolteae and any combination thereof. In some embodiments, the bacterium is a drug resistant bacterium that is resistant to at least one antibiotic. In some embodiments, the bacterium is a multi-drug resistant bacterium that is resistant to at least one antibiotic. In some embodiments, the bacterium is Pseudomonas. In some embodiments, the bacterium is staphylococcus. In some embodiments, the bacterium is Escherichia coli. In some embodiments, the bacterium is Clostridium difficile. In some embodiments, the bacterium is methicillin resistant. In some embodiments, the bacterium is methicillin resistant Staphylococcus aureus. In some embodiments, the bacterium is multidrug resistant Pseudomonas Aeruginosa. In some embodiments, the antibiotic comprises a cephalosporin, a fluoroquinolone, a carbapenem, a colistin, an aminoglycoside, vancomycin, streptomycin, or methicillin. In some embodiments, the administering is intra-arterial, intravenous, intramuscular, oral, subcutaneous, topical, inhalation, or any combination thereof.

Disclosed herein, in certain embodiments, are bacteriophages comprising: lytic activity, and a first nucleic acid sequence encoding an anti-CRISPR polypeptide. In specific embodiments, the anti-CRISPR polypeptide enhances the lytic activity of the bacteriophage. In some embodiments, the anti-CRISPR polypeptide inactivates a CRISPR-Cas system. In some embodiments, the anti-CRISPR polypeptide inactivates the CRISPR-Cas system using a process comprising gene regulation interference. In some embodiments, the anti-CRISPR polypeptide inactivates the CRISPR-Cas system using a process comprising nuclease recruitment interference. In some embodiments, the CRISPR-Cas system is a type I CRISPR-Cas system, a type II CRISPR-Cas system, or a type III CRISPR-Cas system. In some embodiments, the CRISPR-Cas system is a type I CRISPR-Cas system. In some embodiments, the anti-CRISPR polypeptide binds directly or indirectly to a Cascade or Cascade-like complex. In some embodiments, the anti-CRISPR polypeptide is a truncated protein, a fusion protein, a dimer protein, or mutated protein. In some embodiments, the bacteriophage further comprises a second nucleic acid encoding a CRISPR array. In some embodiments, CRISPR array comprises at least one repeat sequence and at least one spacer sequence that is complementary to a target nucleotide sequence from a target gene in the target bacterium. Also disclosed herein, in some embodiments, are pharmaceutical compositions comprising the bacteriophage disclosed herein, and a pharmaceutically acceptable excipient. In some embodiments, the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof. Further disclosed herein, in some embodiments, are methods of treating a disease in a subject comprising administering the bacteriophage disclosed herein to the subject. In some embodiments, the subject is a mammal. In some embodiments, the disease is a bacterial infection. In some embodiments, a bacterium causing the bacterial infection is an Acinetobacter species, an Actinomyces species, Burkholderia cepacia complex, a Campylobacter species, a Candida species, Clostridium difficile, Corynebacterium minutissium, Corynebacterium pseudodiphtheriae, Corynebacterium stratiurn, Corynebacterium group G1, Corynebacterium group G2, Enterobacteriaceae, an Enterococcus species, Escherichia coli, Haemophilus influenzae, Klebsiella pneumoniae, a Moraxella species, Mycobacterium tuberculosis complex, Neisseria gonorrhoeae, Neisseria meningitidis, a non-tuberculous mycobacteria species, a Porphyromonas species, Prevotella melaninogenicus, a Pseudomonas species, Salmonella typhimurium, Serratia marcescens Staphylococcus aureus, Streptococcus agalactiae, Staphylococcus epidermidis, Staphylococcus salivarius, Streptococcus mitis, Streptococcus sanguis, Streptococcus pneumoniae, Streptococcus pyogenes, Vibrio cholerae, a Coccidioides species, a Cryptococcus species, Helicobacter felis, Helicobacter pylori, Clostridium bolteae and any combination thereof. In some embodiments, the bacterium is a drug resistant bacterium that is resistant to at least one antibiotic. In some embodiments, the bacterium is a multi-drug resistant bacterium that is resistant to at least one antibiotic. In some embodiments, the bacterium is Pseudomonas. In some embodiments, the bacterium is staphylococcus. In some embodiments, the bacterium is Escherichia coli. In some embodiments, the bacterium is Clostridium difficile. In some embodiments, the bacterium is methicillin resistant. In some embodiments, the bacterium is methicillin resistant Staphylococcus aureus. In some embodiments, the bacterium is multidrug resistant Pseudomonas Aeruginosa. In some embodiments, the antibiotic comprises a cephalosporin, a fluoroquinolone, a carbapenem, a colistin, an aminoglycoside, vancomycin, streptomycin, or methicillin. In some embodiments, the administering is intra-arterial, intravenous, intramuscular, oral, subcutaneous, topical, inhalation, or any combination thereof.

Disclosed herein, in certain embodiments, are methods of killing a target bacterium, comprising introducing into a target bacterium a temperate bacteriophage comprising a removal, replacement, or inactivation of at least one lysogeny gene, wherein the temperate bacteriophage is rendered lytic thereby killing the target bacterium. In some embodiments, the lysogeny gene is a repressor gene. In some embodiments, the lysogeny gene is cI phage repressor gene. In some embodiments, the bacteriophage infects multiple bacterial strains. In some embodiments, the target bacterium is C. difficile. In some embodiments, the bacteriophage is ϕCD146 or ϕCD24-2. In some embodiments, the bacteriophage further comprises a first nucleic acid encoding a spacer sequence or a crRNA transcribed therefrom. In some embodiments, the spacer sequence is complementary to a target nucleotide sequence from a target gene in the target bacterium. In some embodiments, the first nucleic acid sequence is a CRISPR array further comprising at least one repeat sequence. In some embodiments, the at least one repeat sequence is operably linked to the spacer sequence at either its 5′ end or its 3′ end. In some embodiments, the bacteriophage further comprises a second nucleic acid encoding a transcriptional activator for a CRISPR-Cas system. In some embodiments, the CRISPR-Cas system is endogenous to the target bacterium. In some embodiments, the CRISPR-Cas system is exogenous to the target bacterium. In some embodiments, the CRISPR-Cas system is a type I CRISPR-Cas system, a type II CRISPR-Cas system, or a type III CRISPR-Cas system. In some embodiments, the CRISPR-Cas system comprises a type I CRISPR-Cas system. In some embodiments, the target nucleotide sequence comprises all or a part of a promoter sequence for the target gene. In some embodiments, the target nucleotide sequence comprises all or a part of a nucleotide sequence located on a coding strand of a transcribed region of the target gene. In some embodiments, the target nucleotide sequence comprises at least a portion of an essential gene that is needed for survival of the target bacterium. In some embodiments, the target bacterium is killed by both lytic activity of the bacteriophage and activity of the CRISPR-Cas system in combination. In some embodiments, activity of the CRISPR-Cas system supplements or enhances lytic activity of the bacteriophage. In some embodiments, the target bacterium is killed by activity of a CRISPR-Cas system independently of lytic activity of the bacteriophage. In some embodiments, lytic activity of the bacteriophage and activity of a CRISPR-Cas system are synergistic. In some embodiments, lytic activity of the bacteriophage, activity of a CRISPR-Cas system, or both is modulated by a concentration of the bacteriophage.

Disclosed herein, in certain embodiments, are pharmaceutical compositions comprising: (a) a temperate bacteriophage comprising a removal, replacement, or inactivation of at least one lysogeny gene; and (B) a pharmaceutically acceptable excipient. In some embodiments, the pharmaceutical composition is in a form of a tablet, a liquid, a syrup, an oral formulation, an intravenous formulation, an intranasal formulation, an ocular formulation, an otic formulation, a subcutaneous formulation, an inhalable respiratory formulation, a suppository, and any combination thereof.

Disclosed herein, in certain embodiments, are methods of tuning the microbiome of a subject, the method comprising: administering to the subject a pharmaceutical composition disclosed herein. In some embodiments, a pharmaceutical composition disclosed herein treats microbiome imbalance.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 illustrates the workflow process for engineering a CRISPR-enhanced bacteriophage.

FIG. 2A exemplifies a schematic diagram of the linear alignment of CRISPR-Cas systems from within five strains of C. difficile with identification of the various CRISPR-Cas constituent components.

FIG. 2B further exemplifies a schematic diagram of the CRISPR-Cas system operon structures for C. difficile strains 630 and R20291.

FIG. 3A exemplifies the reduction in cell population for E. coli strain BW25113 when treated with a native wild-type bacteriophage T7m or a corresponding engineered crPhage T7m. Native wild-type bacteriophage shows bacteria killing by lytic activity. Corresponding crT7m comprising LeuO and a CRISPR array for ftsA shows an additional 5-log improvement in bacterial killing activity over the wild-type bacteriophage T7m.

FIG. 3B exemplifies the lethality of the CRISPR array for ftsA (a 7-log reduction) when administered directly to the bacteria independent of phage delivery.

FIG. 4A exemplifies the reduction in cell population for C. difficile strain R20291 when treated with a native wild-type bacteriophage ϕCD146 or the corresponding engineered crPhage ϕCD146. Native wild-type bacteriophage shows bacteria killing by lytic activity. However, the corresponding crPhage ϕCD146 shows an additional 1-log improvement in bacterial killing activity over the wild-type bacteriophage.

FIG. 4B exemplifies the lethality of the CRISPR array for R20291-3 (a 3.5-log reduction) when administered directly to the bacteria independent of phage delivery.

FIG. 5 exemplifies a bacterial lawn of C. difficile strain 069 which are insensitive to lysis by wild-type phage ϕCD146. The lack of phage plaques on the left of the image indicates lack of killing by the wild-type phage ϕCD146. The presence of plaques due to crPhage ϕCD146 on the right of the image is indicative of bacterial death due to the activity of the CRISPR array targeting R20291-3.

FIG. 6A-FIG. 6V exemplify the enhanced lethality of crPhage ϕCD146 as compared to the wild-type bacteriophage against different strains of C. difficile. FIG. 6A exemplifies crPhage ϕCD146 lethality against C. difficile strain 043. Population growth of the various strains of C. difficile was monitored by optical density at 600 nm for up to 6 hours following treatment with either the wild-type or CRISPR-array containing crPhage ϕCD146. FIG. 6B exemplifies crPhage ϕCD146 lethality against C. difficile strain 051. FIG. 6C exemplifies crPhage ϕCD146 lethality against C. difficile strain 073. FIG. 6D exemplifies crPhage ϕCD146 lethality against C. difficile strain 093. FIG. 6E exemplifies crPhage ϕCD146 lethality against C. difficile strain 0180. FIG. 6F exemplifies crPhage ϕCD146 lethality against C. difficile strain 106. FIG. 6G exemplifies crPhage ϕCD146 lethality against C. difficile strain 128. FIG. 6H exemplifies crPhage ϕCD146 lethality against C. difficile strain 199. FIG. 6I exemplifies crPhage ϕCD146 lethality against C. difficile strain 111. FIG. 6J exemplifies crPhage ϕCD146 lethality against C. difficile strain 108. FIG. 6K exemplifies crPhage ϕCD146 lethality against C. difficile strain 25. FIG. 6L exemplifies crPhage ϕCD146 lethality against C. difficile strain 148. FIG. 6M exemplifies crPhage ϕCD146 lethality against C. difficile strain 154. FIG. 6N exemplifies crPhage ϕCD146 lethality against C. difficile strain 195. FIG. 6O exemplifies crPhage ϕCD146 lethality against C. difficile strain FOBT195. FIG. 6P exemplifies crPhage ϕCD146 lethality against C. difficile strain 038. FIG. 6Q exemplifies crPhage ϕCD146 lethality against C. difficile strain 112. FIG. 6R exemplifies crPhage ϕCD146 lethality against C. difficile strain 196. FIG. 6S exemplifies crPhage ϕCD146 lethality against C. difficile strain 105. FIG. 6T exemplifies crPhage ϕCD146 lethality against C. difficile strain UK1. FIG. 6U exemplifies crPhage ϕCD146 lethality against C. difficile strain UK6. FIG. 6V exemplifies crPhage ϕCD146 lethality against C. difficile strain BI-9.

FIG. 7A-FIG. 7C exemplify the enhanced lethality of crPhage ϕCD24-2 as compared to the wild-type bacteriophage against different strains of C. difficile. Population growth of the various strains of C. difficile was monitored by optical density at 600 nm for up to 6 hours following treatment with either the wild-type or CRISPR-array containing crPhage ϕCD24-2. FIG. 7A exemplifies crPhage ϕCD146 lethality against C. difficile strain 041. FIG. 7B exemplifies crPhage ϕCD146 lethality against C. difficile strain 042. FIG. 7C exemplifies crPhage ϕCD146 lethality against C. difficile strain 046.

FIG. 8A-FIG. 8E exemplify the enhanced lethality of crPhage ϕCD146 as compared to the wild-type bacteriophage against different strains of C. difficile. Population growth of the various strains of C. difficile was monitored using CFU reduction assay for up to 6 hours following treatment with either the wild-type or CRISPR-array containing crPhage ϕCD146. CFU reduction assays provide enhanced quantitative sensitivity over optical density measurements. FIG. 8A exemplifies crPhage ϕCD146 lethality against C. difficile strain 043. FIG. 8B exemplifies crPhage ϕCD146 lethality against C. difficile strain 051. FIG. 8C exemplifies crPhage ϕCD146 lethality against C. difficile strain 073. FIG. 8D exemplifies crPhage ϕCD146 lethality against C. difficile strain 093. FIG. 8E exemplifies crPhage ϕCD146 lethality against C. difficile strain R20291.

FIG. 9 exemplifies the enhanced lethality of crPhage ϕCD24-2 as compared to the wild-type bacteriophage against C. difficile strain F19. Population growth of C. difficile strain CD19 was monitored using CFU reduction assay for up to 6 hours following treatment with either the wild-type or CRISPR-array containing crPhage ϕCD24-2. The use of CFU reduction assay provides enhanced quantitative sensitivity over optical density measurements.

FIG. 10 exemplifies a combinatorial comparison of crPhage ϕCD146 and crPhage ϕCD24-2 tested against C. difficile strain 043. A CFU assay of crPhage ϕCD146 and crPhage ϕCD24-2 anti-bacterial activity was conducted for each crPhage individually as well as the when administered together. Co-administration showed improved killing efficacy as compared to treatment with a combination of both wild-type phages together.

FIG. 11 exemplifies that the killing activity of the crPhage ϕCD146 consistently outperforms the lethality of the wild-type phage over a wide range of viral titer MOIs with a CRISPR array targeting R20291-3.

FIG. 12A-FIG. 12B exemplify an in-silico model predicting the number of resistant clones that emerge over time due to target site mutation as a function of the number of independent genes targeted by crRNAs. These models assume highly conservative assumptions that (1) mutational rate is independent of gene target and (2) that all 32 bases of crRNA match for activity. Two types of infection were modeled: an acute infection rising to a total burden of 10¹⁰ CFU by doubling every 6 hours as seen in FIG. 12A or an aggressive infection to a total burden of 10¹⁴ CFU by doubling every 20 minutes as seen in FIG. 12B. Both models show that 3 independent gene targets are sufficient to prevent mutational escape up to 28 days of infection length.

FIG. 13 exemplifies the strain coverage for a series of individual Type I-E crRNAs targeted to conserved regions of the E. coli genome. crRNA array targets include the following genes in order of highest to lowest percentage of strain coverage: Tsf (100%), acpP (99%), gapA (99%), infA (99%), secY (99%), secY′2 (99%), csrA (99%), trmD (99%), ftsA (99%), nusG (99%), fusA′2 (99%), fusA (98%), glyQ (98%), eno (95%), gapA′2 (91%), eno′2 (89%), and nusG′2 (73%).

FIG. 14 exemplifies the functional lethality assessment for a series of individual type I-E crRNAs with spacers targeted to conserved regions of the E. coli genome. crRNA targets include the following genes: acpP, csrA, eno, fusA, gapA, glyQ, infA, nusG, secY, trmD, and Tsf.

FIG. 15 illustrates a schematic overview of three engineered CRISPR-enhanced bacteriophages against E. coli developed as a cocktail of three distinct obligate lytic bacteriophages that contain an identical DNA sequence encoding the transcriptional activator LeuO and a CRISPR-array. The three engineered LeuO enhanced bacteriophages include crT4, crT7, and crT7m.

FIG. 16A exemplifies the relative prevalence and distribution of canonical Type I-E or Type I-F CRISPR-Cas systems in E. coli. Six hundred and twenty-five publicly available E. coli genomes were analyzed, spanning a diversity of strains including: uropathogenic E. coli (UPEC), Shiga toxin producing E. coli, (STEC), O157:H7 serotype E. coli, diarrheagenic E. coli (DEC), non-157 O antigen type E. coli, and enteropathogenic E. coli (EPEC).

FIG. 16B shows that approximately 78% (487/625) of all tested strains in FIG. 16A have a complete CRISPR-Cas3 system, either type I-E or type I-F.

FIG. 17 exemplifies non-lytic M13-derived phagemid delivery of LeuO enhanced CRISPR array constructs using a validated ftsA spacer sequence designed to test the dependence on LeuO expression for CRISPR-mediated lethality. Lethality of LeuO enhanced phagemid vectors was tested via transduction of M13 bacteriophages into a range of strains including a parent EMG2 containing a wild-type H-NS repressed E. coli Type I-E CRISPR-Cas3 operon, a BW25113-derivative lacking the H-NS repression motifs in the CRISPR-Cas3 operon (Δhns), a BW25113-derivative containing an overexpressed CRISPR-Cas3 operon (BW+Cas) and a BW25113-derivative lacking Cas3 genes (BWΔCas).

FIG. 18A-FIG. 18C exemplify the improved lethality kinetics for the three LeuO enhanced crPhages (FIG. 18A—crT7m, FIG. 18B—crT4, and FIG. 18C—crT7) comprising the transcriptional activator LeuO along with a CRISPR array as compared to their wild-type variants. Target E. coli were incubated for 2 or 5 hours for crT7m, crT4 and crT7, respectively, in growth media at the indicated multiplicity-of-infection (ratio of phage to bacteria) for each phage. Significant differences were observed in CFU reduction across all three crPhages.

FIG. 19A-FIG. 19E exemplify dose-response in vitro kill curves for LeuO enhanced crPhages crT4, crT7, and crT7m against E. coli strain MG1655 for each crPhage or crPhage cocktail and resultant changes in population were measured by optical density. E. coli was grown to mid-log phase and treated with multiplicity-of-infection (MOI; ratio of phage to bacteria) as follows: FIG. 19A, crT7 was incubated at MOIs of 0.0001, 0.01, and 1.0; FIG. 19B, crT7m was incubated at MOIs of 0.0009, 0.09, and 9.0; and FIG. 19C, crT4 was incubated at MOIs of 0.0006, 0.06, and 6.0. Each phage was mixed in equal amounts to create a crPhage cocktail (‘Cocktail’) and was incubated at MOIs (for each crPhage) of 0.0006, 0.06, and 6.0, as seen in FIG. 19D. FIG. 19E is a zoomed in graph from FIG. 19D.

FIG. 20 exemplifies the dose-dependent relationship observed between concentrations of LeuO enhanced crPhages and the resultant time-to-lysis in E. coli MG1655. E. coli was grown to mid-log phase and treated with multiplicity-of-infection (MOI; ratio of phage to bacteria) as indicated for each crPhage. For all three crPhages tested, MOI in excess of 1.0 resulted in the fastest time-to-lysis, presumably being limited by the lytic period of each phage. The observed time-to-lysis for each phage was approximately 15-20 minutes for crT7m and crT7 and approximately 45-50 minutes for crT4.

FIG. 21A-FIG. 21G illustrate a schematic timeline representation of the dosing parameters for an in vivo tolerability of the three LeuO enhanced crPhages crT4, crT7, and crT7m. No overt toxicity was observed during veterinary observation and no measurable changes in body temperature or body weight were noted after dosing with each crPhage preparation as shown in FIG. 21B-FIG. 21G. FIG. 21B and FIG. 21E illustrates crT7 body temperature and body weight after dosing, respectively. FIG. 21C and FIG. 21F illustrates crT7M body temperature and body weight after dosing, respectively. FIG. 21D and FIG. 21G illustrates crT4 body temperature and body weight after dosing, respectively.

FIG. 22A-FIG. 22D illustrate a schematic timeline representation for the treatment parameters for a murine in-vivo peritonitis model with E. coli with the LeuO enhanced crPhages and the results. Female CD-1 mice were injected intraperitoneally with a lethal dose of E. coli (˜5×10⁷ CFU/mouse of ATCC 8739) followed within 30 minutes by intraperitoneal injections of saline or crPhages. Single-dose administration of crPhage (2.0×10¹¹ PFU/dose of crT7 (FIG. 22B), 3.7×10⁹ PFU/dose of crT7m (FIG. 22C) or 6.0×10⁸ PFU/dose of crT4 (FIG. 22D)) resulted in significant protection.

FIG. 23A-FIG. 23E illustrate a schematic timeline representation for the treatment parameters for a murine in-vivo thigh infection model with E. coli treated with the LeuO enhanced crPhages for monitoring the effect upon bacterial bioburden reduction and the results. Mice were inoculated with 10⁵ CFU of E. coli MG1655 by intramuscular injection into the thigh 30 minutes prior to intramuscular injection with the indicated crPhage or crPhage cocktail at corresponding doses of 4.0×10¹¹ PFU/dose of crT7, 2.0×10¹¹ PFU/dose of crT7M, 2.0×10¹⁰ PFU/dose of crT4 or the cocktail containing 1.0×10¹⁰ PFU/dose of each phage. After injection with each crPhage, whole thigh muscles were excised at the indicated time points, homogenized and immediately diluted and plated to count surviving bacterial colonies per gram of tissue. CFU reductions measured approximately 2-log for crT4 (FIG. 23C), 3-log for crT7M (FIG. 23D), and >5-log for both crT7 (FIG. 23B) and the combined crPhage cocktail (FIG. 23E).

FIG. 24 illustrates in-vivo persistence and distribution of LeuO enhanced crPhages. Female CD-1 mice were treated with approximately 1.0×10⁹ PFU/dose/phage of a crT7/crT7m cocktail by intraurethral instillation directly into the bladder. At time points of 0, 0.5, 1, 6, 12, 24 and 72 hours post-inoculation, 3 mice per time point were sacrificed and collected bladder, kidney, blood, liver and spleen whole tissue homogenates were diluted and subjected to phage titration analysis to quantify the total combined amount of crT7 and crT7m. Presence of active crPhage was detected up to 72 hours after dosing. crPhage levels decreased over time in bladder and were undetectable in kidney, liver, blood and spleen by 72 hours. Significant phage titers were observed in the kidneys suggesting that intraurethral route of administration, in some instances, results in exposure in the lower and upper urinary tract. Also, phage titers were detected in blood, liver and spleen tissues, showing that crPhages appear to enter circulation by crossing the urothelium.

FIG. 25A-FIG. 25C exemplify a small scale in-vivo persistence and distribution study of LeuO enhanced crPhages similar to that seen in FIG. 24 using quantitative PCR detection instead of PFU assay. A single dose of 2.7×10⁹ PFU total of each crT7, crT7m and crT4 was administered by oral gavage. Treated mice were sacrificed and total DNA from whole tissue homogenates was extracted and subjected to qPCR analysis to quantify the amount of crT7 (FIG. 25A), crT4 (FIG. 25B) or crT7m (FIG. 25C) present.

FIG. 26A-FIG. 26B exemplify reduction in lysogeny formation rate and reduction in viable CD19 cells treated with a cI-knockout bacteriophage. FIG. 26A exemplifies a reduction in viable CD19 cells when treated with ΔcI CD24-2 as compared to CD19 cells treated with WT CD24-2. FIG. 26B exemplifies a reduction in the percent lysogens in the surviving CD19 cells that were treated with ΔcI CD24-2 as compared to in CD19 cells treated with WT CD24-2.

FIG. 27A-FIG. 27B exemplify comparative CFU reduction in bladder via i.v. delivery (FIG. 27A) or local delivery (FIG. 27B) of wild type phage (wtPhage), crPhage and ciprofloxacin. Results exemplify improved CFU reduction with crPhage compared to wtPhage.

FIG. 28A-FIG. 28B exemplify dose response of crPhage treatment in bladder (FIG. 28A) and in kidney (FIG. 28B). crPhage were delivered intraurethrally.

FIG. 29 illustrates a schematic of an exemplary UTI efficacy study with research-grade material compared WT versus engineered cocktail via intravenous (IV), intra-urethral (IU), or concurrent IV and IU delivery.

FIG. 30A-FIG. 30D exemplify a UTI efficacy study demonstrating reduction in E. coli in bladder (FIG. 30A and FIG. 30B) and kidney (FIG. 30C and FIG. 30D) following intraurethral (IU) or intravenous (IV) administration. Measurements were taken 54 hour post infection in FIG. 30A and FIG. 30C. Measurements were taken 102 hour post infection in FIG. 30B and FIG. 30D. The results exemplify that crPhage cocktail has 1.5 to 3.5-log improved kill over wtPhage cocktail at 120 h in the bladder. The results also exemplify that regardless of delivery route, at 120 h crPhage cocktail performs comparable with ciprofloxacin in the bladder.

FIG. 31A-FIG. 31D illustrate route-dependent penetration of phage into different tissues, such as in urine 78 hour post infection (FIG. 31A), into kidney 102 hour post infection (FIG. 31B), into bladder 102 hour post infection (FIG. 31C), and into spleen 102 hour post infection (FIG. 31D).

FIG. 32 illustrates a schematic of an exemplary UTI efficacy study with research-grade material compared WT versus engineered cocktail via intravenous (IV), intra-urethral (IU), or concurrent IV and IU delivery.

FIG. 33A-FIG. 33D illustrate IV dosing of crPhage cocktail requires high doses for efficacy, while IU delivery is effective even at low doses and high dose crPhage outperforms ciprofloxacin. CFUs in FIG. 33A (bladder) and FIG. 33C (kidney) are analyzed 54 hour post infection. CFUs in FIG. 33B (bladder) and FIG. 33D (kidney) are analyzed 102 hour post infection.

FIG. 34A is a schematic of an exemplary human study conducted in adults with reoccurring and asymptomatic E. coli colonization of the urinary tract.

FIG. 34B is an exemplary study participant inclusion and exclusion criteria for the UTI Phase 1b study.

FIG. 35A-FIG. 35F illustrate engineered phage (p33s and p33s-6) show increased killing against both Type IE and Type IF E. coli strains.

FIG. 36A-FIG. 36F illustrate engineered phage (CRISPR phage crp0046) show increased killing against both Type IE and Type IF E. coli strains.

FIG. 37A-FIG. 37C illustrate switching phage cocktails overcomes target bacterial resistance in E. coli.

FIG. 38A-FIG. 38B illustrate a comparison of wildtype phage PB1 and CRISPR-enhanced PB1 (cr-PB1) against P. aeruginosa strains.

FIG. 39A-FIG. 39B illustrate plasmid based killing in E. coli and P. aeruginosa by Type I CRISPR-Cas systems.

### DETAILED DESCRIPTION

### Definitions

Unless otherwise defined, all technical and scientific terms used herein have the same meaning as commonly understood by one of ordinary skill in the art to which this disclosure belongs. The terminology used in the description of the disclosure herein is for the purpose of describing particular embodiments only and is not intended to be limiting of the disclosure.

Unless the context indicates otherwise, it is specifically intended that the various features of the disclosure described herein are able of being used in any combination. Moreover, the present disclosure also contemplates that in some embodiments, any feature or combination of features set forth herein are excluded or omitted. To illustrate, if the specification states that a composition comprises components A, B and C, it is specifically intended that any of A, B or C, or a combination thereof, are omitted and disclaimed singularly or in any combination.

One of skill in the art will understand the interchangeability of terms designating the various CRISPR-Cas systems and their components due to a lack of consistency in the literature and an ongoing effort in the art to unify such terminology. Likewise, one of skill in the art will also understand the interchangeability of terms designating the various anti-CRISPR proteins due to a lack of consistency in the literature and an ongoing effort in the art to unify such terminology.

As used in the description and the appended claims, the singular forms “a,” “an” and “the” are intended to include the plural forms as well, unless the context clearly indicates otherwise. Also as used herein, “and/or” refers to and encompasses any and all possible combinations of one or more of the associated listed items, as well as the lack of combinations when interpreted in the alternative (“or”).

The term “about” as used herein when referring to a measurable value such as a dosage or time period and the like refers to variations of ±20%, ±10%, ±5%, ±1%, +0.5%, or even ±0.1% of the specified amount. As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

The term “comprise”, “comprises”, and “comprising”, “includes”, “including”, “have” and “having”, as used herein, specify the presence of the stated features, steps, operations, elements, and/or components, but do not preclude the presence or addition of one or more other features, steps, operations, elements, components, and/or groups thereof.

As used herein, the transitional phrase “consisting essentially of” means that the scope of a claim is to be interpreted to encompass the specified materials or steps recited in the claim and those that do not materially affect the basic and novel characteristic(s) of the claimed disclosure. Thus, the term “consisting essentially of” when used in a claim of this disclosure is not intended to be interpreted to be equivalent to “comprising.”

The term “consists of” and “consisting of”, as used herein, excludes any features, steps, operations, elements, and/or components not otherwise directly stated. The use of “consisting of” limits only the features, steps, operations, elements, and/or components set forth in that clause and does exclude other features, steps, operations, elements, and/or components from the claim as a whole.

As used herein, “chimeric” refers to a nucleic acid molecule or a polypeptide in which at least two components are derived from different sources (e.g., different organisms, different coding regions).

“Complement” as used herein mean 100% complementarity or identity with the comparator nucleotide sequence or it mean less than 100% complementarity (e.g., about 80%, 81%, 82%, 83%, 84%, 85%, 86%, 87%, 88%, 89%, 90%, 91%, 92%, 93%, 94%, 95%, 96%, 97%, 98%, 99%, and the like, complementarity). Complement or complementable may also be used in terms of a “complement” to or “complementing” a mutation.

The terms “complementary” or “complementarity”, as used herein, refer to the natural binding of polynucleotides under permissive salt and temperature conditions by base-pairing. For example, the sequence “A-G-T” binds to the complementary sequence “T-C-A.” Complementarity between two single-stranded molecules is “partial,” in which only some of the nucleotides bind, or it is complete when total complementarity exists between the single stranded molecules. The degree of complementarity between nucleic acid strands has significant effects on the efficiency and strength of hybridization between nucleic acid strands.

As used herein, the term “gene” refers to a nucleic acid molecule capable of being used to produce mRNA, tRNA, rRNA, miRNA, anti-microRNA, regulatory RNA, and the like. Genes may or may not be capable of being used to produce a functional protein or gene product. Genes include both coding and non-coding regions (e.g., introns, regulatory elements, promoters, enhancers, termination sequences and/or 5′ and 3′ untranslated regions). A gene is “isolated” by which is meant a nucleic acid that is substantially or essentially free from components normally found in association with the nucleic acid in its natural state. Such components include other cellular material, culture medium from recombinant production, and/or various chemicals used in chemically synthesizing the nucleic acid.

As used herein, a “target nucleotide sequence” refers to the portion of a target gene that is complementary to the spacer sequence of the recombinant CRISPR array.

As used herein, a “target DNA,” “target nucleotide sequence,” “target region,” or a “target region in the genome” refers to a region of an organism's genome that is fully complementary or substantially complementary (e.g., at least 70% complementary (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a spacer sequence in a CRISPR array. In some embodiments, a target region is about 10 to about 40 consecutive nucleotides in length located immediately adjacent to a PAM sequence (PAM sequence located immediately 3′ of the target region) in the genome of the organism. In some embodiments, a target nucleotide sequence is located adjacent to or flanked by a PAM (protospacer adjacent motif). While PAMs are often specific to the particular CRISPR— Cas system, a PAM sequence is determined by a suitable method. Thus, for example, experimental approaches include targeting a sequence flanked by all possible nucleotides sequences and identifying sequence members that do not undergo targeting, such as through in vitro cleavage of target DNA or the transformation of target plasmid DNA. In some embodiments, a computational approach includes performing BLAST searches of natural spacers to identify the original target DNA sequences in bacteriophages or plasmids and aligning these sequences to determine conserved sequences adjacent to the target sequence.

As used herein, the term “protospacer adjacent motif” or “PAM” refers to a DNA sequence present on the target DNA molecule adjacent to the sequence matching the guide RNA spacer. This motif is found in the target gene next to the region to which a spacer sequence binds as a result of being complementary to that region and identifies the point at which base pairing with the spacer nucleotide sequence begins. For type I systems, the PAM is located immediately 5′ to the sequence that matches the spacer, and thus is 3′ to the sequence that base pairs with the spacer nucleotide sequence. Non-limiting examples of PAMs include CCA, CCT, CCG, CCT, CCA, TTC, AAG, AGG, ATG, GAG, and/or CC. For type I systems, PAM is directly recognized by Cascade. The exact PAM sequence that is required varies between each different CRISPR-Cas system and is identified through established bioinformatics and experimental procedures. Once a protospacer is recognized, Cascade generally recruits the endonuclease Cas3, which cleaves and degrades the target DNA.

For type II systems, the PAM is required for a Cas9/sgRNA to form an R-loop to interrogate a specific DNA sequence through Watson-Crick pairing of its guide RNA with the genome. The PAM specificity is a function of the DNA-binding specificity of the Cas9 protein (e.g., a—protospacer adjacent motif recognition domain at the C-terminus of Cas9).

As used herein, type I Clustered Regularly Interspaced Short Palindromic Repeats (CRISPR)-associated complex for antiviral defense (Cascade) refers to a complex of polypeptides involved in processing of pre-crRNAs and subsequent binding to the target DNA in type I CRISPR-Cas systems. These polypeptides include, but are not limited to, the Cascade polypeptides of type I subtypes 1-A, 1-B, 1-C, 1-D, 1-E and 1-F. Non-limiting examples of type I-A polypeptides include Cas7 (Csa2), Cas8a1 (Csx13), Cas8a2 (Csx9), Cas5, Csa5, Cas6a, Cas3′ and/or a Cas3″. Non-limiting examples of type 1-B polypeptides include Cas6b, Cas8b (Csh1), Cas7 (Csh2) and/or Cas5. Non-limiting examples of type-IC polypeptides include Cas5d, Cas8c (Csd1), and/or Cas7 (Csd2). Non-limiting examples of type-ID polypeptides include Cas10d (Csc3), Csc2, Csc1, and/or Cas6d. Non-limiting examples of type 1-E polypeptides include Cse1 (CasA), Cse2 (CasB), Cas7 (CasC), Cas5 (CasD) and/or Cas6e (CasE). Non-limiting examples of type I-F polypeptides include Cys1, Cys2, Cas7 (Cys3) and/or Cas6f (Csy4). In some embodiments, a recombinant nucleic acid described herein comprises, consists essentially of, or consists of, a nucleotide sequence encoding a subset of type-1 Cascade polypeptides that function to process a CRISPR array and subsequently bind to a target DNA using the spacer of the processed CRISPR RNA as a guide.

A “CRISPR array” as used herein means a nucleic acid molecule that comprises at least two repeat sequences, or a portion of each of said repeat sequences, and at least one spacer sequence. One of the two repeat sequences, or a portion thereof, is linked to the 5′ end of the spacer sequence and the other of the two repeat sequences, or portion thereof, is linked to the 3′ end of the spacer sequence. In a recombinant CRISPR array, the combination of repeat sequences and spacer sequences is synthetic, made by man and not found in nature. In some embodiments, a “CRISPR array” refers to a nucleic acid construct that comprises from 5′ to 3′ at least one repeat-spacer sequences (e.g., about 1, 2, 3, 4, 5, 6, 7, 8, 9, 10, 11, 12, 13, 14, 15, 16, 17, 18, 19, 20, 21, 22, 23, 24, 25 or more repeat-spacer sequences, and any range or value therein), wherein the 3′ end of the 3′ most repeat-spacer sequence of the array are linked to a repeat sequence, thereby all spacers in said array are flanked on both the 5′ end and the 3′ end by a repeat sequence.

As used herein, “spacer sequence” or “spacer refers to a nucleotide sequence that is complementary to a target DNA (i.e., target region in the genome or the “protospacer sequence,” which is adjacent to a protospacer adjacent motif (PAM) sequence). The spacer sequence is fully complementary or substantially complementary (e.g., at least about 70% complementary (e.g., about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to a target DNA.

A “repeat sequence” as used herein, refers to, for example, any repeat sequence of a wild-type CRISPR locus or a repeat sequence of a synthetic CRISPR array that are separated by “spacer sequences” (e.g., a repeat-spacer-repeat sequence). A repeat sequence useful with this disclosure is any known or later identified repeat sequence of a CRISPR locus or it is a synthetic repeat designed to function in a CRISPR system, for example CRISPR Type I system.

As used herein, the term “CRISPR phage”, “CRISPR enhanced phage”, and “crPhage” refers to a bacteriophage particle comprising bacteriophage DNA comprising at least one heterologous polynucleotide. In some embodiments, the polynucleotide encodes at least one component of a CRISPR-Cas system (e.g., CRISPR array, crRNA; e.g., PI bacteriophage comprising an insertion of crRNA targeting). In some embodiments, the polynucleotide encodes at least one transcriptional activator of a CRISPR-Cas system. In some embodiments, the polynucleotide encodes at least one component of an anti-CRISPR polypeptide of a CRISPR-Cas system.

As used herein, the phrase “substantially identical,” or “substantial identity” in the context of two nucleic acid molecules, nucleotide sequences or protein sequences, refers to two or more sequences or subsequences that have at least about 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and/or 100% nucleotide or amino acid residue identity, when compared and aligned for maximum correspondence, as measured using one of the following sequence comparison algorithms or by visual inspection. In some embodiments, substantial identity refer to two or more sequences or subsequences that have at least about 70%, at least about 75%, at least about 80%, at least about 85%, at least about 90%, at least about 95, 96, 96, 97, 98, or 99% identity. For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for aligning a comparison window are conducted by tools such as the local homology algorithm of Smith and Waterman, the homology alignment algorithm of Needleman and Wunsch, the search for similarity method of Pearson and Lipman, and optionally by computerized implementations of these algorithms such as GAP, BESTFIT, FASTA, and TFASTA available as part of the GCG® Wisconsin Package® (Accelrys Inc., San Diego, Calif.). An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence. Percent sequence identity is represented as the identity fraction multiplied by 100. The comparison of one or more polynucleotide sequences is to a full-length polynucleotide sequence or to a portion thereof, or to a longer polynucleotide sequence. In some instances, “Percent identity” is determined using BLASTX version 2.0 for translated nucleotide sequences and BLASTN version 2.0 for polynucleotide sequences.

In some embodiments, the recombinant nucleic acids molecules, nucleotide sequences and polypeptides disclosed herein are “isolated.” An “isolated” nucleic acid molecule, an “isolated” nucleotide sequence or an “isolated” polypeptide is a nucleic acid molecule, nucleotide sequence or polypeptide that exists apart from its native environment. In some instances, an isolated nucleic acid molecule, nucleotide sequence or polypeptide exists in a purified form that is at least partially separated from at least some of the other components of the naturally occurring organism or virus, for example, the cell or viral structural components or other polypeptides or nucleic acids commonly found associated with the polynucleotide. In representative embodiments, the isolated nucleic acid molecule, the isolated nucleotide sequence and/or the isolated polypeptide is at least about 1%, 5%, 10%, 20%, 30%, 40%, 50%, 60%, 70%, 80%, 90%, 95%, or more pure.

As used herein, the terms “anti-CRISPR” or “Acr” refers to any protein or gene product with functional anti-CRISPR activity. Due to a lack of consistency in the literature, one of skill in the art will understand the interchangeability of terms designating the various anti-CRISPR proteins. For example, as used herein the designation of Acr1-Bo is interchangeable with AcrIIC1Boe and the designation of Acr2-Nm is interchangeable with AcrIIC2Nme. Also, as used herein, the designation of Acr88a-32 is interchangeable with AcrE2. An anti-CRISPR protein is any bacteriophage protein with activity that prevents the function of a bacterial CRISPR-Cas system. Activity of an anti-CRISPR protein prevents a host bacterium from mounting a CRISPR-Cas system based defense against the invading bacteriophage.

By the terms “treat,” “treating,” or “treatment,” it is intended that the severity of the subject's condition is reduced or at least partially improved or modified and that some alleviation, mitigation or decrease in at least one clinical symptom is achieved, and/or there is a delay in the progression of the disease or condition, and/or delay of the onset of a disease or illness. With respect to an infection, a disease or a condition, the term refers to a decrease in the symptoms or other manifestations of the infection, disease or condition. In some embodiments, treatment provides a reduction in symptoms or other manifestations of the infection, disease or condition by at least about 5%, e.g., about 10%, 15%, 20%, 25%, 30%, 35%, 40%, 45%, 50% or more.

The terms with respect to an “infection”, “a disease”, or “a condition”, used herein, refer to any adverse, negative, or harmful physiological condition in a subject. In some embodiments, the source of an “infection”, “a disease”, or “a condition”, is the presence of a target bacterial population in and/or a subject. In some embodiments, the bacterial population comprises one or more target bacterial species. In some embodiments, the one or more bacteria in the bacterial population comprise one or more strains of one or more bacteria. In some embodiments, the target bacterial population causing an “infection”, “a disease”, or “a condition” is acute or chronic. In some embodiments, the target bacterial population causing an “infection”, “a disease”, or “a condition” is localized or systemic. In some embodiments, the target bacterial population causing an “infection”, “a disease”, or “a condition” is idiopathic. In some embodiments, the target bacterial population causing an “infection”, “a disease”, or “a condition” is acquired through means, including but not limited to, respiratory inhalation, ingestion, skin and wound infections, blood stream infections, middle-ear infections, gastrointestinal tract infections, peritoneal membrane infections, urinary tract infections, urogenital tract infections, oral soft tissue infections, intra-abdominal infections, epidermal or mucosal absorption, eye infections (including contact lens contamination), endocarditis, infections in cystic fibrosis, infections of indwelling medical devices such as joint prostheses, dental implants, catheters and cardiac implants, sexual contact, and/or hospital-acquired and ventilator-associated bacterial pneumonias.

As used herein the term “biofilm” means an accumulation of microorganisms embedded in a matrix of polysaccharide. Biofilms form on solid biological or non-biological surfaces and are medically important, accounting for over 80 percent of microbial infections in the body.

The terms “prevent,” “preventing,” and “prevention” (and grammatical variations thereof) refer to prevention and/or delay of the onset of an infection, disease, condition and/or a clinical symptom(s) in a subject and/or a reduction in the severity of the onset of the infection, disease, condition and/or clinical symptom(s) relative to what would occur in the absence of carrying out the methods disclosed herein prior to the onset of the disease, disorder and/or clinical symptom(s). Thus, in some embodiments, to prevent infection, food, surfaces, medical tools and devices are treated with compositions and by methods disclosed herein.

A “subject” disclosed herein includes any animal that has or is susceptible to an infection, disease or condition involving bacteria. Thus, in some embodiments, subjects are mammals, avians, reptiles, amphibians, or fish. Mammalian subjects include but are not limited to humans, non-human primates (e.g., gorilla, monkey, baboon, and chimpanzee, etc.), dogs, cats, goats, horses, pigs, cattle, sheep, and the like, and laboratory animals (e.g., rats, guinea pigs, mice, gerbils, hamsters, and the like). Avian subjects include but are not limited to chickens, ducks, turkeys, geese, quail, pheasants, and birds kept as pets (e.g., parakeets, parrots, macaws, cockatoos, canaries, and the like). In some embodiments, suitable subjects include both males and females and subjects of any age, including embryonic (e.g., in-utero or in-ovo), infant, juvenile, adolescent, adult and geriatric subjects. In some embodiments, a subject is a human.

By “pharmaceutically acceptable” it is meant a material that is not biologically or otherwise undesirable, i.e., the material are administered to a subject without causing any undesirable biological effects such as toxicity.

Provided in various embodiments are bacteriophages, characterized and/or comprising any nucleic acid described herein. In some embodiments, provided herein is a temperate bacteriophage (e.g., a bacteriophage having lytic activity, such as described herein). In specific embodiments, the temperate bacteriophage comprises a removal, replacement, or inactivation of at least one lysogeny gene. In certain embodiments, provided herein are bacteriophages having lytic activity and comprising (a) a nucleic acid encoding a spacer sequence and/or (b) a crRNA transcribed therefrom. In some specific embodiments, provided in certain embodiments herein are bacteriophages comprising (i) a first nucleic acid encoding a spacer sequence and/or a crRNA transcribed therefrom, and (ii) a gene that is capable of inducing lysis of a bacterium (e.g., a target bacterium). In more specific embodiments, the spacer sequence is complementary to a nucleic acid sequence of a target gene of or in a bacterium (e.g., target bacterium). Provided in some embodiments herein are bacteriophages comprising a nucleic acid encoding a transcriptional activator for the CRISPR-Cas system in a bacterium (e.g., a target bacterium). In certain embodiments, provided herein are bacteriophages having lytic activity and a first nucleic acid sequence encoding an anti-CRISPR polypeptide (and/or comprising an anti-CRISPR polypeptide). In various embodiments, a bacteriophage provided herein has any one or more of the above references characteristics and/or activities. Moreover, in various embodiments herein, such bacteriophages comprise any one or more characteristic or activity described in the summary or detailed description herein.

In some embodiments, such bacteriophages are utilized and various compositions (e.g., pharmaceutical compositions) and methods, such as described herein. In certain embodiments, such bacteriophages are useful in any number of applications and methods (e.g., of tuning the microbiome of an individual or subject, such as one in need thereof), such as those described herein. In some embodiments, such bacteriophages are utilized in methods of or that involve killing (e.g., selectively killing) a bacterium (e.g., a target bacterium). In specific embodiments, the target bacterium is in a mixed population of bacteria, such as in an individual, environment, or other suitable location, such as described herein.

In certain embodiments, a bacteriophage provided herein selectively kills a target bacteria or bacterium, e.g., such that the bacteria that is not the target bacterium or bacteria is killed at a lesser rate than the target bacteria, such as at less than 50% the rate, less than 25% the rate, less than 10% the rate, or about 0% the rate (i.e., not at all) relative to the target bacterium or bacteria. In some instances, such as in certain methods provided herein, less than 50% of the non-target bacterium is killed, less than 25%, less than 20%, less than 10%, less than 5% killed, or the like is killed.

### CRISPR Array

Disclosed herein are CRISPR arrays. In some embodiments, a nucleic acid encoding a CRISPR array comprises at least one repeat sequence and at least one spacer sequences complementary to a target nucleotide sequence from a target gene in the target bacterium. In some embodiments, a CRISPR array is of any length and comprises any number of spacer nucleotide sequences alternating with repeat nucleotide sequences necessary to achieve the desired level of killing of the target bacterium by use of one or more target genes. In some embodiments, the CRISPR array comprise, consist essentially of, or consist of 1 to about 100 spacer nucleotide sequences, each linked on its 5′ end and its 3′ end to a repeat nucleotide sequence. In some embodiments, a recombinant CRISPR array of disclosed herein, consist essentially of, or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

Spacer

In some embodiments, the spacer sequence described herein comprises one, two, three, four, or five mismatches as compared to the target DNA. In some embodiments, mismatches are contiguous. In some embodiments, mismatches are noncontiguous. In some embodiments, the spacer sequence has 70% complementarity to a target DNA. In some embodiments, the spacer nucleotide sequence has 80% complementarity to a target DNA. In some embodiments, the spacer nucleotide sequence is 85%, 90%, 95%, 96%, 97%, 98%, 99% or 100% complementarity to a target nucleotide sequence of a target gene. In some embodiments, the spacer sequence has 100% complementarity to the target DNA. In some embodiments, a spacer sequence has complete complementarity or substantial complementarity over a region of a target nucleotide sequence that are at least about 8 nucleotides to about 150 nucleotides in length. In some embodiments, a spacer sequence have complete complementarity or substantial complementarity over a region of a target nucleotide sequence that is at least about 20 nucleotides to about 100 nucleotides in length. In some embodiments, the 5′ region of a spacer sequence is 100% complementary to a target DNA while the 3′ region of the spacer is substantially complementary to the target DNA and therefore the overall complementarity of the spacer sequence to the target DNA is less than 100%. For example, in some embodiments, the first 7, 8, 9, 10, 11, 12, 13, 14, 15, 16 nucleotides in the 3′ region of a 20 nucleotide spacer sequence (seed region) is 100% complementary to the target DNA, while the remaining nucleotides in the 5′ region of the spacer sequence are substantially complementary (e.g., at least about 70% complementary) to the target DNA. In some embodiments, the first 7 to 12 nucleotides of the 3′ end of the spacer sequence is 100% complementary to the target DNA, while the remaining nucleotides in the 5′ region of the spacer sequence are substantially complementary (e.g., at least about 50% complementary (e.g., 50%, 55%, 60%, 65%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or more)) to the target DNA. In some embodiments, the first 7 to 10 nucleotides in the 3′ end of the spacer sequence is 75%-99% complementary to the target DNA, while the remaining nucleotides in the 5′ region of the spacer sequence are at least about 50% to about 99% complementary to the target DNA. In some embodiments, the first 7 to 10 nucleotides in the 3′ end of the spacer sequence is 100% complementary to the target DNA, while the remaining nucleotides in the 5′ region of the spacer sequence are substantially complementary (e.g., at least about 70% complementary) to the target DNA. In some embodiments, the first 10 nucleotides (within the seed region) of the spacer sequence is 100% complementary to the target DNA, while the remaining nucleotides in the 5′ region of the spacer sequence are substantially complementary (e.g., at least about 70% complementary) to the target DNA. In some embodiment, the 5′ region of a spacer sequence (e.g., the first 8 nucleotides at the 5′ end, the first 10 nucleotides at the 5′ end, the first 15 nucleotides at the 5′ end, the first 20 nucleotides at the 5′ end) have about 75% complementarity or more (75% to about 100% complementarity) to a target DNA, while the remainder of the spacer sequence have about 50% or more complementarity to the target DNA. In some embodiments, the first 8 nucleotides at the 5′ end of a spacer sequence have 100% complementarity to the target nucleotide sequence or have one or two mutations and therefore is about 88% complementary or about 75% complementary to a target DNA, respectively, while the remainder of the spacer nucleotide sequence is at least about 50% or more complementary to the target DNA.

In some embodiments, a spacer sequence described herein is about 15 nucleotides to about 150 nucleotides in length. In some embodiments, a spacer nucleotide sequence is about 15 nucleotides to about 100 nucleotides in length (e.g., about 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 nucleotides or more). In some embodiments, a spacer nucleotide sequence is a length of about 8 to about 150 nucleotides, about 8 to about 100 nucleotides, about 8 to about 50 nucleotides, about 8 to about 40 nucleotides, about 8 to about 30 nucleotides, about 8 to about 25 nucleotides, about 8 to about 20 nucleotides, about 10 to about 150 nucleotides, about 10 to about 100 nucleotides, about 10 to about 80 nucleotides, about 10 to about 50 nucleotides, about 10 to about 40, about 10 to about 30, about 10 to about 25, about 10 to about 20, about 15 to about 150, about 15 to about 100, about 15 to about 50, about 15 to about 40, about 15 to about 30, about 20 to about 150 nucleotides, about 20 to about 100 nucleotides, about 20 to about 80 nucleotides, about 20 to about 50 nucleotides, about 20 to about 40, about 20 to about 30, about 20 to about 25, at least about 8, at least about 10, at least about 15, at least about 20, at least about 25, at least about 30, at least about 32, at least about 35, at least about 40, at least about 44, at least about 50, at least about 60, at least about 70, at least about 80, at least about 90, at least about 100, at least about 110, at least about 120, at least about 130, at least about 140, at least about 150 nucleotides in length, or more, and any value or range therein.

In some embodiments, the identity of two or more spacer nucleotide sequences of a CRISPR array disclosed herein is the same. In some embodiments, the identity of two or more spacer nucleotide sequences of a CRISPR array disclosed herein is different. In some embodiments, the identity of two or more spacer nucleotide sequences of a CRISPR array is different but are complementary to one or more target nucleotide sequences. In some embodiments, the identity of two or more spacer nucleotide sequences of a CRISPR array is different and are complementary to one or more target nucleotide sequences that are overlapping sequences. In some embodiments, the identity of two or more spacer nucleotide sequences of a CRISPR array is different and are complementary to one or more target nucleotide sequences that are not overlapping sequences.

Codon Optimization

In some embodiments, a polynucleotide, nucleotide sequence and/or recombinant nucleic acid molecule described herein (e.g., polynucleotides comprising a CRISPR array, Cascade polypeptides, Cas9 polypeptides, Cas3 polypeptides, Cas3′ polypeptides, Cas3″ polypeptides, recombinant Type I or Type II, Type III, Type IV, Type V, Type VI CRISPR-Cas systems of the disclosure, polynucleotides encoding transcriptional activators, and the like) is codon optimized for expression in any species of interest. Codon optimization involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. Codon optimization of a nucleotide sequence results in a nucleotide sequence having less than 100% identity (e.g., 50%, 60%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to the native nucleotide sequence but which still encodes a polypeptide having the same function as that encoded by the original nucleotide sequence. In some embodiments, the nucleotide sequence and/or recombinant nucleic acid molecule of this disclosure are codon optimized for expression in the organism/species of interest.

Repeat Nucleotide Sequences

In some embodiments, a repeat nucleotide sequence of a CRISPR array comprises a nucleotide sequence of any known repeat nucleotide sequence of a CRISPR-Cas system. In some embodiment, the CRISPR-Cas system is a type-I CRISPR-Cas system. In some embodiment, a repeat nucleotide sequence is of a synthetic sequence comprising the secondary structure of a native repeat from a type-I CRISPR-Cas system (e.g., an internal hairpin).

In some embodiments, a spacer nucleotide sequence of a CRISPR array described herein is linked at its 5′ end to the 3′ end of a repeat sequence. In some embodiments, the spacer nucleotide sequence is linked at its 5′ end to about 1 to about 8, about 1 to about 10, or about 1 to about 15 nucleotides of the 3′ end of a repeat nucleotide sequence. In some embodiments, the about 1 to about 8, about 1 to about 10, about 1 to about 15 nucleotides of the repeat nucleotide sequence are a portion of the 3′ end of a repeat nucleotide sequence. In some embodiments, spacer nucleotide sequence is linked at its 3′ end to the 5′ end of a repeat nucleotide sequence. In some embodiments, the spacer is linked at its 3′ end to about 1 to about 8, about 1 to about 10, or about 1 to about 15 nucleotides of the 5′ end of a repeat nucleotide sequence. In some embodiments, the about 1 to about 8, about 1 to about 10, about 1 to about 15 nucleotides of the repeat nucleotide sequence are a portion of the 5′ end of a repeat nucleotide sequence.

In some embodiments, a spacer nucleotide sequence described herein is linked at its 5′ end to a first repeat nucleotide sequence and linked at its 3′ end to a second repeat nucleotide sequence to form a repeat-spacer-repeat sequence. In some embodiments, a spacer described herein is linked at its 5′ end to about 1 to about 8, about 1 to about 10, or about 1 to about 15 nucleotides of the 3′ end of a first repeat sequence and is linked at its 3′ end to about 1 to about 8, about 1 to about 10, or about 1 to about 15 nucleotides of the 5′ end of a second repeat sequence. In some embodiments, the about 1 to about 8, about 1 to about 10, about 1 to about 15 nucleotides of the first repeat sequence are a portion of the 3′ end of the first repeat nucleotide sequence. In some embodiments, the about 1 to about 8, about 1 to about 10, about 1 to about 15 nucleotides of the first second sequence are a portion of the 3′ end of the second repeat nucleotide sequence. In some embodiments, a spacer nucleotide sequence disclosed herein is linked at its 5′ end to the 3′ end of a first repeat nucleotide sequence and is linked at its 3′ end to the 5′ of a second repeat nucleotide sequence where the spacer nucleotide sequence and the second repeat nucleotide sequence are repeated to form a repeat-(spacer-repeat)n sequence such that n is any integer from 1 to 100. Thus, in some embodiments, a repeat-(spacer-repeat)n sequence disclosed herein comprise, consist essentially of, or consist of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more, spacer nucleotide sequences.

Thus, in some embodiments, a repeat sequence is identical to or substantially identical to a repeat sequence from a wild-type CRISPR Type I, II, or III loci. In some embodiments, a repeat sequence comprises a portion of a wild type repeat sequence (e.g., 1, 2, 3, 4, 5, 6, 7, 8, 9, 10, 11, 12, 13, 14, 15 or more contiguous nucleotides of a wild type repeat sequence). In some embodiments, a repeat sequence comprises, consists essentially of, or consists of at least one nucleotide (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 45, 50, 55, 60, 65, 70, 75, 80, 85, 90, 95, 100, or more nucleotides, or any range therein).

Regulatory Elements

In some embodiments, recombinant CRISPR arrays, nucleotide sequences, and/or nucleic acid molecules disclosed herein are operatively associated with a variety of promoters, terminators and other regulatory elements for expression in various organisms or cells. In some embodiments, at least one promoter and/or terminator is operably linked to a recombinant nucleic acid molecule and/or a recombinant CRISPR array disclosed herein. Any promoter useful with this disclosure is used and includes, for example, promoters functional with the organism of interest as well as constitutive, inducible, developmental regulated, tissue-specific/preferred-promoters, and the like, as described herein. A regulatory element as used herein is endogenous or heterologous. In some embodiments, an endogenous regulatory element derived from the subject organism is inserted into a genetic context in which it does not naturally occur (e.g. a different position in the genome than as found in nature), thereby producing a recombinant or non-native nucleic acid.

In some embodiments, expression of a construct disclosed herein is constitutive, inducible, temporally regulated, developmentally regulated, or chemically regulated. In some embodiments, a construct is made constitutive, inducible, temporally regulated, developmentally regulated, or chemically regulated by operatively linking the construct to a promoter functional in an organism of interest. In some embodiments, repression is made reversible by operatively linking a recombinant nucleic acid construct disclosed herein to an inducible promoter that is functional in an organism of interest. The choice of promoter described herein will vary depending on the quantitative, temporal and spatial requirements for expression, and also depending on the host cell to be transformed.

Exemplary promoters for use with the methods, bacteriophage and composition disclosed herein include promoters that are functional in bacteria. For example, L-arabinose inducible (araBAD, P_{BAD}) promoter, any lac promoter, L-rhamnose inducible (rhaPBAD) promoter, T7 RNA polymerase promoter, trc promoter, tac promoter, lambda phage promoter (p_{L}p_{L}-9G-50), anhydrotetracycline-inducible (tetA) promoter, trp, Ipp, phoA, recA, proU, cst-1, cadA, nar, Ipp-lac, cspA, 11-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, α-amylase (Pamy), Ptms, P43 (comprised of two overlapping RNA polymerase G factor recognition sites, σA, σB), Ptms, P43, rplK-rplA, ferredoxin promoter, and/or xylose promoter.

In some embodiments, inducible promoters are used. In some embodiment, chemical-regulated promoters are used to modulate the expression of a gene in an organism through the application of an exogenous chemical regulator. The use of chemically regulated promoters enables RNAs and/or the polypeptides disclosed herein to be synthesized only when, for example, an organism is treated with the inducing chemicals. In some embodiments where a chemical-inducible promoter is used, the application of a chemical induces gene expression. In some embodiments wherein a chemical-repressible promoter is used, the application of the chemical represses gene expression. In some embodiments, the promoter is a light-inducible promoter, where application of specific wavelengths of light induces gene expression. In some embodiments, a promoter is a light-repressible promoter, where application of specific wavelengths of light represses gene expression.

Transformation

In some embodiments, the nucleotide sequences, constructs, and expression cassettes disclosed herein are expressed transiently and/or stably incorporated into the genome of a host organism. In some embodiments, a polynucleotide disclosed herein is introduced into a cell by any method known to those of skill in the art. Exemplary methods of transformation include transformation via electroporation of competent cells, passive uptake by competent cells, chemical transformation of competent cells, as well as any other electrical, chemical, physical (mechanical) and/or biological mechanism that results in the introduction of nucleic acid into a cell, including any combination thereof. In some embodiments, transformation of a cell comprises nuclear transformation. In some embodiments, transformation of a cell comprises plasmid transformation and conjugation.

In some embodiments, when more than one nucleotide sequence is introduced, the nucleotide sequences are assembled as part of a single nucleic acid construct, or as separate nucleic acid constructs, and are located on the same or different nucleic acid constructs. In some embodiments, nucleotide sequences are introduced into the cell of interest in a single transformation event, or in separate transformation events.

Expression Cassette

In some embodiments, a nucleic acid construct is an “expression cassette” or in an expression cassette. As used herein, “expression cassette” means a recombinant nucleic acid molecule comprising a nucleotide sequence of interest (e.g., the recombinant nucleic acid molecules and CRISPR arrays disclosed herein), wherein the nucleotide sequence is operably associated with at least a control sequence (e.g., a promoter). In some embodiments, the expression cassettes are designed to express the recombinant nucleic acid molecules and/or the recombinant CRISPR arrays disclosed herein.

In some embodiments, an expression cassette comprising a nucleotide sequence of interest is chimeric, meaning that at least one of its components is heterologous with respect to at least one of its other components. In some embodiments, an expression cassette is naturally occurring but has been obtained in a recombinant form useful for heterologous expression.

In some embodiments, an expression cassette includes a transcriptional and/or translational termination region (i.e. termination region) that is functional in the selected host cell. In some embodiments, termination regions are responsible for the termination of transcription beyond the heterologous nucleotide sequence of interest and for correct mRNA polyadenylation. In some embodiments, the termination region is native to the transcriptional initiation region, is native to the operably linked nucleotide sequence of interest, is native to the host cell, or is derived from another source (i.e., foreign or heterologous to the promoter, to the nucleotide sequence of interest, to the host, or any combination thereof). In some embodiments, terminators are operably linked to the recombinant nucleic acid molecule and CRISPR array disclosed herein.

In some embodiments, an expression cassette includes a nucleotide sequence for a selectable marker. As used herein, “selectable marker” means a nucleotide sequence that when expressed imparts a distinct phenotype to the host cell expressing the marker and thus allows such transformed cells to be distinguished from those that do not have the marker. In some embodiments, a nucleotide sequence encode either a selectable or screenable marker, depending on whether the marker confers a trait that is selected for by chemical means, such as by using a selective agent (e.g. an antibiotic), or on whether the marker is simply a trait that one identifies through observation or testing, such as by screening (e.g., fluorescence).

Vectors

In addition to expression cassettes, the nucleic acid molecules and nucleotide sequences described herein (e.g. polynucleotides comprising a CRISPR array, polynucleotides encoding a transcriptional activator, or anti-CRISPR polypeptides) are used in connection with vectors. The term “vector” refers to a composition for transferring, delivering or introducing a nucleic acid (or nucleic acids) into a cell. A vector comprises a nucleic acid molecule comprising the nucleotide sequence(s) to be transferred, delivered or introduced. Non-limiting examples of general classes of vectors include but are not limited to a viral vector, a plasmid vector, a phage vector, a phagemid vector, a cosmid vector, a fosmid vector, a bacteriophage, an artificial chromosome, or an agrobacterium binary vector in double or single stranded linear or circular form which may or may not be self-transmissible or mobilizable. A vector as defined herein transforms prokaryotic or eukaryotic host either by integration into the cellular genome or exist extrachromosomally (e.g. autonomous replicating plasmid with an origin of replication). Additionally included are shuttle vectors by which is meant a DNA vehicle capable, naturally or by design, of replication in two different host organisms. In some embodiments, a shuttle vector replicates in actinomycetes and bacteria and/or eukaryotes. In some embodiments, the nucleic acid in the vector are under the control of, and operably linked to, an appropriate promoter or other regulatory elements for transcription in a host cell. In some embodiments, the vector is a bi-functional expression vector which functions in multiple hosts.

### CRISPR/CAS Systems

CRISPR-Cas systems are naturally adaptive immune systems found in bacteria and archaea. The CRISPR system is a nuclease system involved in defense against invading phages and plasmids that provides a form of acquired immunity. There is a diversity of CRISPR-Cas systems based on the set of cas genes and their phylogenetic relationship. There are at least six different types (I through VI) where Type I represents over 50% of all identified systems in both bacteria and archaea. In some embodiments, a Type I, Type II, Type II, Type IV, Type V, or Type VI CRISPR-Cas system is used herein.

In some embodiments, processing of a CRISPR-array disclosed herein includes, but is not limited to, the following processes: 1) transcription of the nucleic acid encoding a CRISPR array into a pre-crRNA and optional tracrRNA; 2) pre-crRNA processing by either Cas6 or Cas9/Rnase III into mature crRNAs; 3) mature crRNA complexation Cas9 or Cascade; 4) target recognition by the complexed mature crRNA/Cas9 or crRNA/Cascade complexes; and 5) nuclease activity at the target leading to double or single stranded DNA breakage.

With regard to Type I systems, Type I systems are divided into seven subtypes including: type I-A, type I-B, type I-C, type I-D, type I-E, type I-F, and type I-U. Type I CRISPR-Cas systems include a multi-subunit complex called Cascade (for complex associated with antiviral defense), Cas3 (a protein with nuclease, helicase, and exonuclease activity that is responsible for degradation of the target DNA), and crRNA (stabilizes Cascade complex and directs Cascade and Cas3 to DNA target). Cascade forms a complex with the crRNA, and the protein-RNA pair recognizes its genomic target by complementary base pairing between the 5′ end of the crRNA sequence and a predefined protospacer. This complex is directed to homologous loci of pathogen DNA via regions encoded within the crRNA and protospacer-adjacent motifs (PAMs) within the pathogen genome. Base pairing occurs between the crRNA and the target DNA sequence leading to a conformational change. In the Type I-E system, the PAM is recognized by the CasA protein within Cascade, which then unwinds the flanking DNA to evaluate the extent of base pairing between the target and the spacer portion of the crRNA. Sufficient recognition leads Cascade to recruit and activate Cas3. Cas3 then nicks the non-target strand and begins degrading the strand in a 3′-to-5′ direction.

In some embodiments, a CRISPR array disclosed herein comprises a nucleic acid that encodes a processed, mature crRNA. In some embodiments, a mature crRNA is introduced into a phage or a target bacterium described herein. In some embodiments, a phage comprises a nucleic acid that encodes a processed, mature crRNA. In some embodiments, an endogenous or exogenous Cas6 processes a CRISPR array into mature crRNA. In some embodiments, an exogenous Cas6 is introduced into a phage. In some embodiments, a phage comprises an exogenous Cas6. In some embodiments, an exogenous Cas6 is introduced into a target bacterium.

In some embodiments, the CRISPR-Cas system is endogenous to the target bacterium. In some embodiments, the CRISPR-Cas system is exogenous to the target bacterium. In some embodiments, the CRISPR-Cas system is a type II CRISPR-Cas system. In some embodiments, the CRISPR-Cas system is a type III CRISPR-Cas system.

In some embodiments, a Type I CRISPR-Cas system is used herein. As described above, Type-I Cascade polypeptides process CRISPR arrays to produce a processed RNA that is then used to bind the complex to a DNA that is complementary to a spacer in the processed RNA. In some embodiments, a first nucleic acid that is introduced into the bacteriophage encodes the Cascade polypeptides that are involved in processing of the first nucleic acid disclosed herein.

In some embodiments, a type-I Cascade polypeptide disclosed herein have an amino acid sequence having substantial identity to a wild-type type-I Cascade polypeptide. In some embodiments, a Cascade polypeptide described herein is a functional fragment of any full length type-1 Cascade polypeptides. In some embodiments, the type I Cascade complex is a type I-A Cascade polypeptides, a type I-B Cascade polypeptides, a type I-C Cascade polypeptides, a type I-D Cascade polypeptides, a type I-E Cascade polypeptides, a type I-F Cascade polypeptides, or a type I-U Cascade polypeptides.

In some embodiments, the type I Cascade complex comprises: (a) a nucleotide sequence encoding a Cas6b polypeptide, a nucleotide sequence encoding a Cas8b (Csh1) polypeptide, a nucleotide sequence encoding a Cas7 (Csh2) polypeptide, and a nucleotide sequence encoding a Cas5 polypeptide (Type I-B); (b) a nucleotide sequence encoding a Cas5d polypeptide, a nucleotide sequence encoding a Cas8c (Csd1) polypeptide, and a nucleotide sequence encoding a Cas7 (Csd2) polypeptide (Type I-C); (c) a nucleotide sequence encoding a Cse1 (CasA) polypeptide, a nucleotide sequence encoding a Cse2 (CasB) polypeptide, a nucleotide sequence encoding a Cas7 (CasC) polypeptide, a nucleotide sequence encoding a Cas5 (CasD) polypeptide, and a nucleotide sequence encoding a Cas6e (CasE) polypeptide (Type I-E); (d) a nucleotide sequence encoding a Cys1 polypeptide, a nucleotide sequence encoding a Cys2 polypeptide, a nucleotide sequence encoding a Cas7 (Cys3) polypeptide, and a nucleotide sequence encoding a Cas6f polypeptide (Type 1-F); (e) a nucleotide sequence encoding a Cas7 (Csa2) polypeptide, a nucleotide sequence encoding a Cas8a1 (Csx13) polypeptide or a Cas8a2 (Csx9) polypeptide, a nucleotide sequence encoding a Cas5 polypeptide, a nucleotide sequence encoding a Csa5 polypeptide, a nucleotide sequence encoding a Cas6a polypeptide, a nucleotide sequence encoding a Cas3′ polypeptide, and a nucleotide sequence encoding a Cas3″ polypeptide having no nuclease activity (Type I-A); and/or (f) a nucleotide sequence encoding a Cas1 Od (Csc3) polypeptide, a nucleotide sequence encoding a Csc2 polypeptide, a nucleotide sequence encoding a Csc1 polypeptide, and a nucleotide sequence encoding a Cas6d polypeptide (Type 1-D).

### Bacteriophages

Bacteriophages or “phages” represent a group of bacterial viruses and are engineered or sourced from environmental sources. Individual bacteriophage host ranges are usually narrow, meaning, phages are highly specific to one strain or few strains of a bacterial species and this specificity makes them unique in their antibacterial action. Bacteriophages are bacterial viruses that rely on the host's cellular machinery to replicate. Generally, phages generally fall into three categories: lytic, lysogenic, and temperate. Lytic bacteriophages infect a host cell, undergo numerous rounds of replication, and trigger cell lysis to release newly made bacteriophage particles. In some embodiments, the lytic bacteriophages disclosed herein retain their replicative ability. In some embodiments, the lytic bacteriophages disclosed herein retain their ability to trigger cell lysis. In some embodiments, the lytic bacteriophages disclosed herein retain both they replicative ability and the ability to trigger cell lysis. In some embodiments, the bacteriophages disclosed herein comprise a CRISPR array. In some embodiments, the CRISPR array does not affect the bacteriophages ability to replicate and/or trigger cell lysis. Lysogenic bacteriophages permanently reside within the host cell, either within the bacterial genome or as an extrachromosomal plasmid. Temperate bacteriophages are capable of being lytic or lysogenic, and choose one versus the other depending on growth conditions and the physiological state of the cell. Anytime a lysogenic bacterium is exposed to adverse conditions, the lysogenic state is terminated. This process is called induction. Adverse conditions which favor the termination of the lysogenic state include desiccation, exposure to UV or ionizing radiation, and exposure to mutagenic chemicals. This leads to the expression of the phage genes, reversal of the integration process, and lytic multiplication.

Bacteriophages package and deliver synthetic DNA using three general approaches. Under the first approach, the synthetic DNA is randomly recombined into the bacteriophage genome, which usually involves a selectable marker. Under the second approach, restriction sites within the phage are used to introduce synthetic DNA in-vitro. Under the third approach, a plasmid generally encoding the phage packaging sites and lytic origin of replication is packaged as part of the assembly of the bacteriophage particle. The resulting plasmids have been coined “phagemids.”

Phages are limited to a given bacterial strain for evolutionary reasons. In some cases, injecting their genetic material into an incompatible strain is counterproductive. Phages have therefore evolved to specifically infect a limited cross-section of strains. However, some phages have been discovered that inject their genetic material into a wide range of bacteria. The classic example is the PI phage, which has been shown to inject DNA in a range of gram-negative bacteria.

In some embodiments, the bacteriophage or phagemid DNA is from a lysogenic or temperate bacteriophage. In some embodiments, the bacteriophage or phagemid DNA is from an obligate lytic bacteriophage. In some embodiments, the bacteriophages or phagemids include but are not limited to PI phage, a M1 3 phage, a λ phage, a T4 phage, a ϕC2 phage, a ϕCD27 phage, a ϕNM1 phage, Bc431 v3 phage, ϕ10 phage, ϕ25 phage, ϕ151 phage, A511-like phages, B054, 0176-like phages, or Campylobacter phages (such as NCTC 12676 and NCTC 12677). In some embodiments, the bacteriophage is ϕCD146 C. difficile bacteriophage. In some embodiments, the bacteriophage is ϕCD24-2 C. difficile bacteriophage. In some embodiments, the bacteriophage is T4 E. coli bacteriophage. In some embodiments, the bacteriophage is T7 E. coli bacteriophage. In some embodiments, the bacteriophage is T7m E. coli bacteriophage.

In some embodiments, a plurality of bacteriophages are used together. In some embodiments, the plurality of bacteriophages used together targets the same or different bacteria within a sample or subject. In some embodiments, the bacteriophages used together comprises T4 phage, T7 phage, T7m phage, or any combination of bacteriophages described herein.

In some embodiments, bacteriophages of interest are obtained from environmental sources. or commercial research vendors. In some embodiments, obtained bacteriophages are screened for lytic activity against a library of bacteria and their associated strains. In some embodiments, the bacteriophages are screened against a library of bacteria and their associated strains for their ability to generate primary resistance in the screened bacteria.

In some embodiments, disclosed herein are method for killing a target bacterium comprising introducing into a target bacterium a bacteriophage comprising: a nucleic acid encoding a spacer sequence or a crRNA transcribed therefrom, wherein the spacer sequence is complementary to a target nucleotide sequence from a target gene in the target bacterium; and a gene that is capable of inducing lysis of the target bacterium, wherein the target bacterium is killed by lytic activity of the bacteriophage or activity of a CRISPR-Cas system using the spacer sequence or the crRNA transcribed therefrom. In some embodiments, disclosed herein are bacteriophages comprising: a nucleic acid encoding a spacer sequence or a crRNA transcribed therefrom, wherein the spacer sequence is complementary to a target nucleotide sequence from a target gene in a target bacterium; and a gene that is capable of inducing lysis of the target bacterium, wherein the target bacterium is killed by the lytic activity of the bacteriophage or activity of a CRISPR-Cas system using the spacer sequence or the crRNA transcribed therefrom.

Insertion Sites

In some embodiments, the introduction of a nucleic acid encoding a CRISPR array into a bacteriophage does not disrupt the lytic activity of the bacteriophage. In some embodiments, the introduction of a nucleic acid encoding a CRISPR array into a bacteriophage preserves the lytic activity of the bacteriophage. In some embodiments, the nucleic acid is inserted into the bacteriophage genome. In some embodiments, the nucleic acid is inserted into the bacteriophage genome at a transcription terminator site at the end of an operon of interest. In some embodiments, the nucleic acid is inserted into the bacteriophage genome as a replacement for one or more removed non-essential genes. In some embodiments, the nucleic acid is inserted into the bacteriophage genome as a replacement for one or more removed lysogenic genes. In some embodiments, the replacement of non-essential and/or lysogenic genes with the nucleic acid does not affect the lytic activity of the bacteriophage. In some embodiments, the replacement of non-essential and/or lysogenic genes with the nucleic acid preserves the lytic activity of the bacteriophage. In some embodiments, the replacement of non-essential and/or lysogenic genes with the nucleic acid enhances the lytic activity of the bacteriophage. In some embodiments, the replacement of non-essential and/or lysogenic genes with the nucleic acid renders a lysogenic bacteriophage lytic.

In some embodiments, the nucleic acid is introduced into the bacteriophage genome at a first location while one or more non-essential and/or lysogenic genes are separately removed and/or inactivated from the bacteriophage genome at a separate location. In some embodiments, the removal and/or inactivation of one or more non-essential and/or lysogenic genes does not affect the lytic activity of the bacteriophage. In some embodiments, the removal and/or inactivation of one or more non-essential and/or lysogenic genes preserves the lytic activity of the bacteriophage. In some embodiments, the removal of one or more non-essential and/or lysogenic genes renders a lysogenic bacteriophage into a lytic bacteriophage. Similarly, in some embodiments, one or more lytic genes are introduced into the bacteriophage so as to render a non-lytic, lysogenic bacteriophage into a lytic bacteriophage.

In some embodiments, the bacteriophage is a temperate bacteriophage which has been rendered lytic by any of the aforementioned means. In some embodiments, a temperate bacteriophage is rendered lytic by the removal, replacement, or inactivation of one or more lysogenic genes. In some embodiments, the lytic activity of the bacteriophage is due to the removal, replacement, or inactivation of at least one lysogeny gene. In some embodiments, a temperate bacteriophage is rendered lytic by the removal, replacement, or inactivation of one or more lysogenic gene and comprises a CRISPR array comprising at least one spacer that is complementary to a target nucleotide sequence in a target gene in a target bacterium. In some embodiments, a temperate bacteriophage is rendered lytic by the removal, replacement, or inactivation of one or more lysogenic gene via a CRISPR array comprising a spacer directed to the one or more lysogenic gene and comprises a CRISPR array comprising at least one spacer that is complementary to a target nucleotide sequence in a target gene in a target bacterium. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, two or more lysogeny genes are removed, replaced, or inactivated to cause arrest of a bacteriophage lysogeny cycle and/or induction of a lytic cycle. In some embodiments, a temperate bacteriophage is rendered lytic by the insertion of one or more lytic genes. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way of introducing an additions CRIPSR array. In some embodiments, the bacteriophage does not confer any new properties onto the target bacterium beyond cellular death cause by lytic activity of the bacteriophage and/or the activity of the CRISPR array.

In some embodiments, the replacement, removal, inactivation, or any combination thereof, of one or more non-essential and/or lysogenic genes is achieved by chemical, biochemical, and/or any suitable method. In some embodiments, the insertion of one or more lytic genes is achieved by any suitable chemical, biochemical, and/or physical method by homologous recombination.

In some embodiments, the bacteriophage is an obligate lytic bacteriophage. In some embodiments, the bacteriophage is ϕCD146 C. difficile bacteriophage. In some embodiments, the bacteriophage is ϕCD24-2 C. difficile bacteriophage. In some embodiments, the bacteriophage is T4 E. coli bacteriophage. In some embodiments, the bacteriophage is T7 E. coli bacteriophage. In some embodiments, the bacteriophage is T7m E. coli bacteriophage.

Non-Essential Gene

In some embodiments, the non-essential gene to be removed and/or replaced from the bacteriophage is a gene that is non-essential for the survival of the bacteriophage. In some embodiments, the non-essential gene to be removed and/or replaced from the bacteriophage is a gene that is non-essential for the induction and/or maintenance of lytic cycle. In some embodiments, the non-essential gene to be removed and/or replaced from the bacteriophage is gp49 from ϕCD146 C. difficile bacteriophage. In some embodiments, the non-essential gene to be removed and/or replaced from the bacteriophage is gp75 from ϕCD24-2C. difficile bacteriophage. In some embodiments, the non-essential gene to be removed and/or replaced from the bacteriophage is the hoc gene from a T4 E. coli bacteriophage. In some embodiments, the non-essential gene to be removed and/or replaced include gp0.7, gp4.3, gp4.5, gp4.7, or any combination thereof from a T7 E. coli bacteriophage. In some embodiments, the non-essential gene to be removed and/or replaced is gp0.6, gp0.65, gp0.7, gp4.3, gp4.5, or any combination thereof from a T7m E. coli bacteriophage.

Transcriptional Activators

In some embodiments, a bacteriophage disclosed herein further comprises a transcriptional activator. In some embodiments, the transcriptional activator encoded regulates the expression of genes of interest within the target bacterium. In some embodiments, the transcriptional activator activates the expression of genes of interest within the target bacterium whether exogenous or endogenous. In some embodiments, the transcriptional activator activates the expression genes of interest within the target bacterium by disrupting the activity of one or more inhibitory elements within the target bacterium. In some embodiments, the inhibitory element comprises a transcriptional repressor. In some embodiments, the inhibitory element comprises a global transcriptional repressor. In some embodiments the inhibitory element is a histone-like nucleoid-structuring (H-NS) protein or homologue or functional fragment thereof. In some embodiments, the inhibitory element is a leucine responsive regulatory protein (LRP). In some embodiments, the inhibitory element is a CodY protein.

In some bacteria, the CRISPR-Cas system is poorly expressed and considered silent under most environmental conditions. In these bacteria, the regulation of the CRISPR-Cas system is the result of the activity of transcriptional regulators, for example histone-like nucleoid-structuring (H-NS) protein which is widely involved in transcriptional regulation of the host genome. H-NS exerts control over host transcriptional regulation by multimerization along AT-rich sites resulting in DNA bending. In some bacteria, such as E. coli, the regulation of the type-I CRISPR-Cas3 operon is regulated by H-NS.

Similarly, in some bacteria, the repression of the CRISPR-Cas system is controlled by an inhibitory element, for example the leucine responsive regulatory protein (LRP). LRP has been implicated in binding to upstream and downstream regions of the transcriptional start sites. Notably, the activity of LRP in regulating expression of the CRISPR-Cas system varies from bacteria to bacteria. Unlike, H-NS which has broad inter-species repression activity, LRP has been shown to differentially regulate the expression of the host CRISPR-Cas system. As such, in some instances, LRP reflects a host-specific means of regulating CRISPR-Cas system expression in different bacteria.

In some instances, the repression of CRISPR-Cas system is also controlled by inhibitory element CodY. CodY is a GTP-sensing transcriptional repressor that acts through DNA binding. The intracellular concentration of GTP acts as an indicator for the environmental nutritional status. Under normal culture conditions, GTP is abundant and binds with CodY to repress transcriptional activity. However, as GTP concentrations decreases, CodY becomes less active in binding DNA, thereby allowing transcription of the formerly repressed genes to occur. As such, CodY acts as a stringent global transcriptional repressor.

In some embodiments, the transcriptional activator is a LeuO polypeptide, any homolog or functional fragment thereof, a nucleic acid sequence encoding the same, or an agent that upregulates LeuO. In some embodiments, the transcriptional activator comprises any ortholog or functional equivalent of LeuO. In some bacteria, LeuO acts in opposition to H-NS by acting as a global transcriptional regulator that responds to environmental nutritional status of a bacterium. Under normal conditions, LeuO is poorly expressed. However, under amino acid starvation and/or reaching of the stationary phase in the bacterial life cycle, LeuO is upregulated. Increased expression of LeuO leads to it antagonizing H-NS at overlapping promoter regions to effect gene expression. Overexpression of LeuO upregulates the expression of the CRISPR-Cas system. In E. coli and S. typhimurium, LeuO drives increased expression of the casABCDE operon which has predicted LeuO and H-NS binding sequences upstream of CasA.

In some embodiments, the expression of LeuO leads to disruption of an inhibitory element. In some embodiments, the disruption of an inhibitory element due to expression of LeuO removes the transcriptional repression of a CRISPR-Cas system. In some embodiments, the expression of LeuO removes transcriptional repression of a CRISPR-Cas system due to activity of H-NS. In some embodiments, the expression of LeuO removes transcriptional repression of a CRISPR-Cas system due to activity of LRP. In some embodiments, the disruption of an inhibitory element due to the expression of LeuO causes an increase in the expression of a CRISPR-Cas system. In some embodiments, the increase in the expression of a CRISPR-Cas system due to the disruption of an inhibitory element caused by the expression of LeuO causes an increase in the CRISPR-Cas processing of a nucleic acid encoding a CRISPR array. In some embodiments, the increase in the expression of a CRISPR-Cas system due to the disruption of an inhibitory element by the expression of LeuO causes an increase in the CRISPR-Cas processing of a nucleic acid encoding a CRISPR array so as to increase the level of lethality of the CRISPR array against a bacterium. In some embodiments, transcriptional activator described herein, causes increase activity of a bacteriophage and/or CRISPR-Cas system described herein.

In some embodiments, the sequence for LeuO or any homolog or functional fragments thereof from E. coli strain K12 includes but is not limited to GenBank accession number: AP0090408.1. The protein sequence for LeuO is listed below in FASTA format as SEQ ID NO. 1:

MPEVQTDHPETAELSKPQLRMVDLNLLTVFDAVMQEQNITRAAHVLGMS
QPAVSNAVARLKVMFNDELFVRYGRGIQPTARAFQLFGSVRQALQLVQN
ELPGSGFEPASSERVFHLCVCSPLDSILTSQIYNHIEQIAPNIHVMFKS
SLNQNTEHQLRYQETEFVISYEDFHRPEFTSVPLFKDEMVLVASKNHPT
IKGPLLKHDVYNEQHAAVSLDRFASFSQPWYDTVDKQASIAYQGMAMMS
VLSVVSQTHLVAIAPRWLAEEFAESLELQVLPLPLKQNSRTCYLSWHEA
AGRDKGHQWMEEQLVSICKR

In some embodiments, the transcriptional activator is a CD2983 polypeptide or any homolog or functional fragment thereof, or a nucleic acid encoding the same. In some embodiments, the transcriptional activator is any ortholog or functional equivalent of CD29883. In some bacteria, CD2983 act as a specific transcriptional regulator that responds to environmental nutritional status of a bacterium. In some bacteria, the CRISPR-Cas system is regulated by the environmental nutritional status of glucose in a ccpA dependent manner. However, under normal conditions CD2983 is suppressed by CodY. Under nutritional stress, CodY becomes less active to allow expression of CD2983. Upregulation of CD2983 is associated with CRISPR-Cas system upregulation.

In some embodiments, the expression of CD2983 leads to disruption of an inhibitory element. In some embodiments, the disruption of an inhibitory element due to expression of CD2983 removes the transcriptional repression of a CRISPR-Cas system. In some embodiments, the expression of CD2983 removes transcriptional repression of a CRISPR-Cas system due to activity of CodY. In some embodiments, the disruption of an inhibitory element due to the expression of CD2983 causes an increase in the expression of a CRISPR-Cas system. In some embodiments, the increase in the expression of a CRISPR-Cas system due to the disruption of an inhibitory element caused by the expression of CD2983 causes an increase in the CRISPR-Cas processing of the first nucleic acid encoding a CRISPR array. In some embodiments, the increase in the expression of a CRISPR-Cas system due to the disruption of an inhibitory element by the expression of CD2983 causes an increase in the CRISPR-Cas processing of the first nucleic acid encoding a CRISPR array so as to increase the level of lethality of the CRISPR array against a bacterium.

In some embodiments, the sequence for CD2983 or any homolog or functional fragments thereof from C. difficile strain 630 includes but is not limited to GenBank accession number: CAJ69877.1. The protein sequence for CD2983 is listed below in FASTA format as SEQ ID NO. 2:

MMILIQSRGKMKCKELSEELEVSERQIKSYKMYLEQAGIFINSTPGIYG
GYEIDKCNSISLIKLLDSEVSILDMINSQLEYNNDIYKNEFNNIVEKIK
AVLNTGEKSDTYMDYFTVQAQRNCDYESEKNKCNEIIRAYTTKHKFWIE
YYSLNSGNSERIVHPYGLFNYKSDTYMVAFCEKRFKFIDFKLCRIKDYK
VLEEKYNVDKSFSWDEYSKNSIGIYKGEEINVVIKISHPFSTIIKEKVW
VNNQQIIEYDDKSIMFKAKMRGYEEIKSWILSMGAYVEVVEPDRLRNDI
LSEIEKMKKIY

In some embodiments, killing of the target bacterium is achieved by the lytic activity of the bacteriophage. In some embodiments, killing of the target bacterium is achieved by the activity of the first nucleic acid encoding a CRISPR array comprising at least one spacer that is complementary to a target nucleotide sequence in a target gene in the target bacterium. In some embodiments, killing of the bacterium is achieved by the processing of the CRISPR array by a type I, type II, or a type III CRISPR-Cas system to produce a processed crRNA capable of directing CRISPR-Cas based endonuclease activity and/or cleavage at the target nucleotide sequence in the target gene of the bacterium. In some embodiments, the killing of the bacterium is achieved by the enhanced processing of the CRISPR array due to the expression of a second nucleic acid encoding a CRISPR-Cas system transcriptional activator.

In some embodiments, killing of the bacterium is achieved by the lytic activity of the bacteriophage and by the activity of a nucleic acid encoding a CRISPR array comprising at least one spacer that is complementary to a target nucleotide sequence in a target gene in the target bacterium in combination. In some embodiments, killing of the bacterium is achieved by the lytic activity of the bacteriophage and by the enhanced activity of the nucleic acid encoding a CRISPR array due to the activity of the expressed transcriptional activator. In some embodiments, the killing of the bacterium by a combination of the lytic activity of the bacteriophage and by the activity of the nucleic acid encoding a CRISPR array is synergistic. In some embodiments, the killing of the bacterium by a combination of the lytic activity of the bacteriophage and by the activity of the nucleic acid encoding a CRISPR array is synergistic due to the expression of the CRISPR-Cas transcriptional activator encoded by the second nucleic acid.

Enhanced Lytic Bacteriophages

Disclosed herein, are methods of producing a bacteriophage that comprises a nucleic acid encoding a transcriptional activator for a CRISPR-Cas system. Also, disclosed herein, are bacteriophages that comprise a nucleic acid encoding a transcriptional activator for a CRISPR-Cas system.

In some embodiments, the introduction of a nucleic acid encoding a transcriptional activator for a CRISPR-Cas into a bacteriophage is used to modulate the activity of a CRISPR-Cas system in the target bacterium. In some embodiments, the transcriptional activator introduced by the bacteriophage increases the expression of a CRISPR-Cas system in the target bacterium. In some embodiments, the increased expression of a CRISPR-Cas system in the target bacterium due to the introduction of a transcriptional activator by a first bacteriophage, enhances the lethality of a second different bacteriophage comprising a CRISPR array as described by previous embodiments. In some embodiments, the increased expression of a CRISPR-Cas system in the target bacterium due to the introduction of a transcriptional activator by a first bacteriophage, enhances the lethality of a second different bacteriophage comprising a pre-processed immature or a processed mature crRNA as described by previous embodiments.

In addition to regulation by transcriptional activators, the CRISPR-Cas system is tightly controlled by other means and mechanisms of regulation. Quorum sensing (QS) is the chemical communication between bacteria within a bacterial population which permits the coordination of gene expression with respect to the population density. QS relies upon chemical signals that are produced and accumulate during bacterial growth. Upon hitting a threshold level, QS signals bind to transcriptional regulators to influence bacterial gene expression. In some bacteria, QS signaling enhances the CRISPR-Cas system for bacterial defense by de-repressing its expression. In addition to QS signaling, the regulation of CRISPR-Cas system expression is believed to be sensitive to perturbations in the host bacterium's membrane integrity. BaeSR is a two component response regulator system that links host membrane envelope stress to the activation of Cas genes. Likewise, heat shock protein G (HtpG) has been shown to stabilize Cas upon induction by phage infection. Additionally, metabolic sensing proteins such as the cAMP metabolite sensing cAMP receptor protein (CRP) are able to activate CRISPR-Cas expression. Other metabolic sensing proteins which regulate Cas expression includes sigma factor RpoN (σ⁵⁴) which responds to nitrogen starvation.

In some embodiments, the transcriptional activator comprises a QS signal. In some embodiments, the transcriptional activator comprises a protein involved in sensing stress to the membrane of the host bacterium. In some embodiments, this protein comprises BaeSR. In some embodiments, the transcriptional activator comprises a protein which stabilizes Cas. In some embodiments, this protein comprises HtpG. In some embodiments, the transcriptional activator is a metabolic sensing protein. In some embodiments, the metabolic sensing protein comprises CRP or RpoN (σ⁵⁴). In some embodiments, a nucleic acid encoding a transcriptional activator or a functional fragment thereof is introduced into the target bacteria. In some embodiments, a nucleic acid encoding a transcriptional activator or a functional fragment thereof is introduced into the target bacteria via a CRISPR array described herein. In some embodiments, the methods disclosed herein comprises: introducing a bacteriophage comprising a nucleic acid encoding a transcriptional activator for the CRISPR-Cas system in the target bacterium. In some embodiments, disclosed herein are bacteriophages comprising a nucleic acid encoding a transcriptional activator for a CRISPR-Cas system in a target bacterium.

### Anti-CRISPR Array

In some embodiments, a bacteriophage disclosed herein further comprises an Anti-CRISPR.

In some embodiments, a method disclosed herein comprises introducing into a target bacterium a bacteriophage comprising: lytic activity, and a first nucleic acid sequence encoding an anti-CRISPR polypeptide, wherein the anti-CRISPR polypeptide enhances the lytic activity of the bacteriophage. In some embodiments, disclosed herein are bacteriophages comprising: lytic activity, and a first nucleic acid sequence encoding an anti-CRISPR polypeptide, wherein the anti-CRISPR polypeptide enhances the lytic activity of the bacteriophage.

In some embodiments, the nucleic acid encoding an anti-CRISPR polypeptide directly enhances the lytic activity of the bacteriophage or another bacteriophage. In some embodiments, enhancement of the lytic activity of the bacteriophage is due to the anti-CRISPR polypeptide inhibiting, inactivating, and/or repressing the activity of a CRISPR-Cas system in the host target bacterium. An anti-CRISPR polypeptide is any bacteriophage protein with activity that prevents the function of a bacterial CRISPR-Cas system. Activity of an anti-CRISPR protein prevents a host bacterium from mounting a CRISPR-Cas system based defense against the invading bacteriophage. In some embodiments, the anti-CRISPR polypeptide inactivates the host bacterium's CRISPR-Cas system using a process comprising gene regulation interference. In some embodiments, the anti-CRISPR polypeptide inactivates the host bacterium's CRISPR-Cas system using a process comprising nuclease recruitment interference. In some embodiments, the anti-CRISPR polypeptide inhibits, inactivates, and/or represses the activity of a type I CRISPR-Cas system, type II CRISPR-Cas system, or a type III CRISPR-Cas system, Type IV CRISPR-Cas system, Type V CRISPR-Cas system, or Type VI CRISPR-Cas system. In some embodiments, the protein product of a nucleic acid encoding an anti-CRISPR polypeptide or the introduced anti-CRISPR polypeptide binds directly or indirectly to a Cascade or a Cascade-like complex.

In some embodiments, the anti-CRISPR polypeptide is a truncated, mutated, or fused to another protein of interest. In some embodiments, the anti-CRISPR polypeptide is a dimer protein. In some embodiments, the anti-CRISPR polypeptide is a homodimer or heterodimer protein. In one embodiment, the anti-CRISPR polypeptide comprises AcrIIC1Boe, AcrIIC1Nme, AcrIIC2Nme, AcrIIC3Nme, AcrIIC4Hpa, AcrIIC5Smu, or any functional fragments thereof. In one embodiment, the anti-CRISPR polypeptide binds with specific affinity to a specific binding site upon the CRISPR-Cas system.

In some embodiments, the anti-CRISPR polypeptide inhibits, inactivates, or represses the activity of a CRISPR-Cas system in the target bacterium, wherein said CRISPR-Cas system targets the bacteriophage comprising the nucleic acid encoding the anti-CRISPR polypeptide. In some embodiments, the anti-CRISPR polypeptide inhibits, inactivates, or represses the activity of a CRISPR-Cas system in the target bacterium, wherein said CRISPR-Cas system targets a second orthogonal bacteriophage different than a first bacteriophage. In some embodiments, the second orthogonal bacteriophage is different than the first bacteriophage. In some embodiments, the inhibition, inactivation, or repression of the CRISPR-Cas system activity in the target bacterium by the anti-CRISPR polypeptide from a first bacteriophage enhances the activity of the first bacteriophage or a second orthogonal bacteriophage. In some embodiments, the second orthogonal bacteriophage has lytic activity. In some embodiments, the second orthogonal bacteriophage comprises a bacteriophage of any of the embodiments disclosed herein.

### Methods of Killing a Target Bacterium

Disclosed herein, in certain embodiments, are methods of killing bacteria. In some embodiments, killing of the target bacterium is achieved by the lytic activity of the bacteriophage. In some embodiments, the lytic activity of the bacteriophage is due to the removal, replacement, or inactivation of at least one lysogeny gene. In some embodiments, the lysogenic gene plays a role in the maintenance of lysogenic cycle in the bacteriophage. In some embodiments, the lysogenic gene plays a role in establishing the lysogenic cycle in the bacteriophage. In some embodiments, the lysogenic gene plays a role in both establishing the lysogenic cycle and in the maintenance of the lysogenic cycle in the bacteriophage. In some embodiments, the lysogenic gene is a repressor gene. In some embodiments, the lysogenic gene is cI repressor gene. In some embodiments, the lysogenic gene is an activator gene. In some embodiments, the lysogenic gene is cII gene. In some embodiments, the lysogenic gene is lexA gene. In some embodiments, the lysogenic gene is int (integrase) gene. In some embodiments, a temperate bacteriophage is rendered lytic by the insertion of one or more genes that contribute to the induction of a lytic cycle. In some embodiments, a temperate bacteriophage is rendered lytic by altering the expression of one or more genes that contribute to the induction of a lytic cycle. In some embodiments, a temperate bacteriophage phenotypically changes from a lysogenic bacteriophage to a lytic bacteriophage. In some embodiments, a temperate bacteriophage is rendered lytic by environmental alterations. In some embodiments, environmental alterations include, but are not limited to, alterations in temperature, pH, or nutrients, exposure to antibiotics, hydrogen peroxide, foreign DNA, or DNA damaging agents, presence of organic carbon, and presence of heavy metal (e.g. in the form of chromium (VI). In some embodiments, a temperate bacteriophage that is rendered lytic is prevented from reverting to lysogenic state. In some embodiments, a temperate bacteriophage that is rendered lytic is prevented from reverting back to lysogenic state by way of introducing an additions CRIPSR array. In some embodiments, killing of a target bacterium is achieved by the activity of a CRISPR array comprising at least one spacer that is complementary to a target nucleotide sequence in a target gene in the target bacterium. In some embodiments, killing of the target bacterium is achieved by the activity of a mature crRNA. In some embodiments, killing of the bacterium is achieved by the processing of the CRISPR array by a Type I, Type II, Type III, Type IV, Type V, or a Type VI CRISPR-Cas system to produce a processed crRNA capable of directing CRISPR-Cas based endonuclease activity and/or cleavage at the target nucleotide sequence in the target gene of the bacterium. In some embodiments, killing of a target bacterium is achieved by the activity of the CRISPR array independent to the lytic and/or non-lytic activity of the bacteriophage. In some embodiments, the killing of a target bacterium is by any method or combination of methods disclosed herein.

In some embodiments, killing of the bacterium are achieved solely by the lytic activity of the bacteriophage. In some embodiments, killing of the bacterium is achieved solely by the activity of the nucleic acid encoding a CRISPR array comprising at least one spacer. In some embodiments, killing of the bacterium is achieved solely by the activity of the nucleic acid encoding a mature crRNA. In some embodiments, killing of the bacterium is achieved by a combination of the lytic activity of the bacteriophage and the activity of the CRISPR array or mature crRNA. In some embodiments, killing of the bacterium by a combination of the lytic activity of the bacteriophage and by the activity of the first nucleic acid encoding a CRISPR array is synergistic. In some embodiments, the killing activity of the CRISPR array or mature crRNA supplements or enhances the lytic activity of the bacteriophage. In some embodiments, killing of a target bacterium is a synergistic effect of two or more systems.

In some embodiments, the synergistic killing of the bacterium is modulated by the concentration of the bacteriophage and/or the design of the CRISPR array. In some embodiments, the synergistic killing of the bacterium is modulated to favor killing by the lytic activity of the bacteriophage over the activity of the CRISPR array by increasing the concentration of bacteriophage administered to the bacterium. In some embodiments, the synergistic killing of the bacterium is modulated to disfavor killing by the lytic activity of the bacteriophage over the activity of the CRISPR array by decreasing the concentration of bacteriophage administered to the bacterium. In some embodiments, at low concentrations, lytic replication allows for amplification and killing of the target bacteria. In some embodiments, at high concentrations, amplification of a phage is not required.

In some embodiments, the synergistic killing of the bacterium is modulated to favor killing by the activity of the CRISPR array over the lytic activity of the bacteriophage by altering the number, the length, the composition, the identity, or any combination thereof, of the spacers so as to increase the lethality of the CRISPR array. In some embodiments, the synergistic killing of the bacterium is modulated to disfavor killing by the activity of the CRISPR array over the lytic activity of the bacteriophage by altering the number, the length, the composition, the identity, or any combination thereof, of the spacers so as to decrease the lethality of the CRISPR array.

In some embodiments, the target nucleotide sequence in the bacterium to be killed is any essential target nucleotide sequence of interest. In some embodiments, the target nucleotide sequence is a non-essential sequence. In some embodiments, a target nucleotide sequence comprises, consists essentially of or consist of all or a part of a nucleotide sequence encoding a promoter, or a complement thereof, of a target gene. In some embodiments, the spacer nucleotide sequence is complementary to a promoter, or a part thereof, of a target gene.

In some embodiments, the target nucleotide sequence comprises all or a part of a nucleotide sequence located on a coding or a non-coding strand of DNA. In some embodiments, the target nucleotide sequence comprises all or a part of a nucleotide sequence located on a coding of a transcribed region of a target gene.

An essential gene is any gene of an organism that is critical for its survival. However, being essential is highly dependent on the circumstances in which an organism lives. For instance, a gene required to digest starch is only essential if starch is the only source of energy. In some embodiments, the essential gene includes but is not limited to: acpP, csrA, eno, fusA, gapA, glyQ, infA, nusG, secY, trmD, Tsf, and ftsA. In some embodiments, a non-essential gene is any gene of an organism that is not critical for survival. However, being non-essential is highly dependent on the circumstances in which an organism lives.

In some embodiments, non-limiting examples of a target gene of interest includes a gene encoding a transcriptional regulator, a translational regulator, a polymerase gene, a metabolic enzyme, a transporter, an RNase, a protease, a DNA replication enzyme, a DNA modifying or degrading enzyme, a regulatory RNA, a transfer RNA, or a ribosomal RNA. In some embodiments, a target gene is a gene involved in cell-division, cell structure, metabolism, motility, pathogenicity or virulence. In some embodiments, a target gene includes a hypothetical gene whose function is not yet characterized. Thus, for example, the target genes are any gene from any bacterium.

Antimicrobial Agents and Peptides

In some embodiments, a bacteriophage disclosed herein is further genetically modified to express an antibacterial peptide, a functional fragment of an antibacterial peptide or a lytic gene. In some embodiments, a bacteriophage disclosed herein express at least one antimicrobial agent or peptide disclosed herein. In some embodiments, a bacteriophage disclosed herein comprises a nucleic acid sequence that encodes an enzybiotic where the protein product of the nucleic acid sequence targets phage resistant bacteria. In some embodiments, the bacteriophage comprises nucleic acids which encode enzymes which assist in breaking down or degrading biofilm matrix. In some embodiments, a bacteriophage disclosed herein comprises nucleic acids encoding Dispersin D aminopeptidase, amylase, carbohydrase, carboxypeptidase, catalase, cellulase, chitinase, cutinase, cyclodextrin glycosyltransferase, deoxyribonuclease, esterase, alpha-galactosidase, beta-galactosidase, glucoamylase, alpha-glucosidase, beta-glucosidase, haloperoxidase, invertase, laccase, lipase, mannosidase, oxidase, pectinolytic enzyme, peptidoglutaminase, peroxidase, phytase, polyphenoloxidase, proteolytic enzyme, ribonuclease, transglutaminase, xylanase or lyase. In some embodiments, the enzyme is selected from the group consisting of cellulases, such as glycosyl hydroxylase family of cellulases, such as glycosyl hydroxylase 5 family of enzymes also called cellulase A; polyglucosamine (PGA) depolymerases; and colonic acid depolymerases, such as 1,4-L-fucodise hydrolase Characterisation of a 1,4-beta-fucoside hydrolase degrading colanic acid, depolymerazing alginase, DNase I, or combinations thereof. In some embodiments, a bacteriophage disclosed herein secretes an enzyme disclosed herein.

In some embodiments, an antimicrobial agent or peptide is expressed and/or secreted by a bacteriophage disclosed herein. In some embodiments, a bacteriophage disclosed herein secretes and expresses an antibiotic such as ampicillin, penicillin, penicillin derivatives, cephalosporins, monobactams, carbapenems, ofloxacin, ciproflaxacin, levofloxacin, gatifloxacin, norfloxacin, lomefloxacin, trovafloxacin, moxifloxacin, sparfloxacin, gemifloxacin, pazufloxacin or any antibiotic disclosed herein. In some embodiments, a bacteriophage disclosed herein comprises a nucleic acid sequence encoding an antibacterial peptide, expresses an antibacterial peptide, or secretes a peptide that aids or enhances killing of a target bacterium. In some embodiments, a bacteriophage disclosed herein comprises a nucleic acid sequence encoding a peptide, a nucleic acid sequence encoding an antibacterial peptide, expresses an antibacterial peptide, or secretes a peptide that aids or enhances the activity of a CRISPR-Cas system.

### Uses

Bacterial Infections

Disclosed herein, are methods of treating bacterial infections. In some embodiments, the bacteriophages disclosed herein treat or prevent diseases or conditions mediated or caused by bacteria as disclosed herein in a human or animal subjects. Such bacteria are typically in contact with tissue of the subject including: gut, oral cavity, lung, armpit, ocular, vaginal, anal, ear, nose or throat tissue. In some embodiments, a bacterial infection is treated by modulating the activity of the bacteria and/or by directly killing of the bacteria.

In some embodiments, one or more target bacteria present in a bacterial population are pathogenic. In some embodiments, the pathogenic bacteria are uropathogenic. In some embodiments, the pathogenic bacterium is uropathogenic E. Coli (UPEC). In some embodiments, the pathogenic bacteria are diarreagenic. In some embodiments, the pathogenic bacteria are diarreagenic E. coli (DEC). In some embodiments, the pathogenic bacteria are Shiga-toxin producing. In some embodiments, the pathogenic bacterium is Shiga-toxin producing E. coli (STEC). In some embodiments, the pathogenic bacteria are Shiga-toxin producing. In some embodiments, the pathogenic bacterium is Shiga-toxin producing E. coli (STEC). In some embodiments, the pathogenic bacterium is Shiga-toxin producing E. coli (STEC). In some embodiments, the pathogenic bacteria are various O-antigen:H-antigen serotype E. coli. In some embodiments, the pathogenic bacteria are entereopathogenic. In some embodiments, the pathogenic bacterium is entereopathogenic E. coli (EPEC).

In some embodiments, the pathogenic bacteria are various strains of C. difficile including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or 820291.

In some embodiments, the bacteriophages disclosed herein are used to treat an infection, a disease, or a condition, in the gastrointestinal tract of a subject. In some embodiments, the bacteriophages are used to modulate and/or kill target bacteria within the microbiome or gut flora of a subject. In some embodiments, the bacteriophages are used to selectively modulate and/or kill one or more target bacteria from a plurality of bacteria within the microbiome or gut flora of a subject. In some embodiments, the bacteriophages are used to selectively modulate and/or kill one or more target entereopathogenic bacteria from a plurality of bacteria within the microbiome or gut flora of a subject. In some embodiments, the target entereopathogenic bacterium is entereopathogenic E. Coli (EPEC). In some embodiments, the bacteriophages are used to selectively modulate and/or kill one or more target diarreagenic bacteria from a plurality of bacteria within the microbiome or gut flora of a subject. In some embodiments, the target diarreagenic bacterium is diarreagenic E. coli (DEC). In some embodiments, the bacteriophages are used to selectively modulate and/or kill one or more target Shiga-toxin producing bacteria from a plurality of bacteria within the microbiome or gut flora of a subject. In some embodiments, the target Shiga-toxin producing bacterium is Shiga-toxin producing E. coli (STEC).

In some embodiments, the bacteriophages are used to selectively modulate and/or kill one or more target enteropathogenic C. difficile bacteria strains within the microbiome or gut flora of a subject including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or R20291.

In some embodiments, the bacteriophages disclosed herein are used to treat an infection, a disease, or a condition, in the urinary tract of a subject. In some embodiments, the bacteriophages are used to modulate and/or kill target bacteria within the urinary tract flora of a subject. The urinary tract flora includes, but is not limited, to Staphylococcus epidermidis, Enterococcus faecalis, and some alpha-hemolytic Streptococci. In some embodiments, the bacteriophages are used to selectively modulate and/or kill one or more target uropathogenic bacteria from a plurality of bacteria within the urinary tract flora of a subject. In some embodiments, the target bacterium is uropathogenic E. Coli (UPEC).

In some embodiments, the bacteriophages disclosed herein are used to treat an infection, a disease, or a condition, on the skin of a subject. In some embodiments, the bacteriophages are used to modulate and/or kill target bacteria on the skin of a subject.

In some embodiments, the bacteriophages disclosed herein are used to treat an infection, a disease, or a condition, on a mucosal membrane of a subject. In some embodiments, the bacteriophages are used to modulate and/or kill target bacteria on the mucosal membrane of a subject.

In some embodiments, the pathogenic bacteria are antibiotic resistant. In one embodiment, the pathogenic bacterium is methicillin-resistant Staphylococcus aureus (MRSA).

In some embodiments, the one or more target bacteria present in the bacterial population form a biofilm. In some embodiments, the biofilm comprises pathogenic bacteria. In some embodiments, the bacteriophage disclosed herein is used to treat a biofilm.

In some embodiments, non-limiting examples of target bacteria includes Escherichia spp., Salmonella spp., Bacillus spp., Corynebacterium Clostridium spp., Clostridium spp., Psuedomonas spp., Clostridium spp., Lactococcus spp., Acinetoibacter spp., Mycobacterium spp., Myxococcus spp., Staphylococcus spp., Streptococcus spp., or cyanobacteria. In some embodiments, non-limiting examples of bacteria include Escherichia coli, Salmonella enterica, Bacillus subtilis, Clostridium acetobutylicum, Clostridium ljungdahlii, Clostridium difficile, Acinetobacter baumannii, Mycobacterium tuberculosis, Myxococcus xanthus, Staphylococcus aureus, Streptococcus pyogenes, or cyanobacteria. In some embodiments, non-limiting examples of bacteria include Staphylococcus aureus, methicillin resistant Staphylococcus aureus, Streptococcus pneumonia, carbapenem-resistant Enteroacteriaceae, Staphylococcus epidermidis, Staphylococcus salivarius, Corynebacterium minutissium, Corynebacterium pseudodiphtheriae, Corynebacterium stratium, Corynebacterium group G1, Corynebacterium group G2, Streptococcus pneumonia, Streptococcus mitis, Streptococcus sanguis, Escherichia coli, Klebsiella pneumoniae, Pseudomonas aeruginosa, Burkholderia cepacia, Serratia marcescens, Haemophilus influenzae, Moraxella sp., Neisseria meningitidis, Neisseria gonorrhoeae, Salmonella typhimurium, Actinomyces spp., Porphyromonas spp., Prevotella melaninogenicus, Helicobacter pylori, Helicobacter fells, or Campylobacter jejuni. Further non-limiting examples of bacteria include lactic acid bacteria including but not limited to Lactobacillus spp. and Bifidobacterium spp.; electrofuel bacterial strains including but not limited to Geobacter spp., Clostridium spp., or Ralstonia eutropha; or bacteria pathogenic on, for example, plants and mammals. In some embodiments, the bacterium is Escherichia coli. In some embodiments, the bacterium is Clostridium difficile.

In some embodiments, the bacteriophage treats acne and other related skin infections.

In some embodiments, a target bacterium is a multiple drug resistant (MDR) bacteria strain. An MDR strain is a bacteria strain that is resistant to at least one antibiotic. In some embodiments, a bacteria strain is resistant to an antibiotic class such as a cephalosporin, a fluoroquinolone, a carbapenem, a colistin, an aminoglycoside, vancomycin, streptomycin, and methicillin. In some embodiments, a bacteria strain is resistant to an antibiotic such as a Ceftobiprole, Ceftaroline, Clindamycin, Dalbavancin, Daptomycin, Linezolid, Mupirocin, Oritavancin, Tedizolid, Telavancin, Tigecycline, Vancomycin, an Aminoglycoside, a Carbapenem, Ceftazidime, Cefepime, Ceftobiprole, a Fluoroquinolone, Piperacillin, Ticarcillin, Linezolid, a Streptogramin, Tigecycline, Daptomycin, or any combination thereof. Examples of MDR strains include: Vancomycin-Resistant Enterococci (VRE), Methicillin-Resistant Staphylococcus aureus (MRSA), Extended-spectrum β-lactamase (ESBLs) producing Gram-negative bacteria, Klebsiella pneumoniae carbapenemase (KPC) producing Gram-negatives, and Multidrug-Resistant gram negative rods (MDR GNR) MDRGN bacteria such as Enterobacter species E. coli, Klebsiella pneumoniae, Acinetobacter baumannii, or Pseudomonas aeruginosa.

In some embodiments the target bacterium is Klebsiella pneumoniae. In some embodiments, the target bacterium is Staphylococcus aureus. In some embodiments, the target bacterium is Enterococci. In some embodiments, the target bacterium is Acinetobacter. In some embodiments, the target bacterium is Pseudomonas. In some embodiments, the target bacterium is Enterobacter. In some embodiments, the target bacterium is Clostridium difficile. In some embodiments, the target bacterium is E. coli. In some embodiments, the target bacterium is Clostridium bolteae. In some embodiments, the methods and compositions disclosed herein are for use in veterinary and medical applications as well as research applications

Microbiome

“Microbiome”, “microbiota”, and “microbial habitat” are used interchangeably hereinafter and refer to the ecological community of microorganisms that live on or in a subject's bodily surfaces, cavities, and fluids. Non-limiting examples of habitats of microbiome include: gut, colon, skin, skin surfaces, skin pores, vaginal cavity, umbilical regions, conjunctival regions, intestinal regions, stomach, nasal cavities and passages, gastrointestinal tract, urogenital tracts, saliva, mucus, and feces. In some embodiments, the microbiome comprises microbial material including, but not limited to, bacteria, archaea, protists, fungi, and viruses. In some embodiments, the microbial material comprises a gram-negative bacterium. In some embodiments, the microbial material comprises a gram-positive bacterium. In some embodiments, the microbial material comprises Proteobacteria, Actinobacteria, Bacteriodetes, or Firmicutes.

In some embodiments, the bacteriophages as disclosed herein are used to modulate or kill target bacteria within the microbiome of a subject. In some embodiments, the bacteriophages are used to modulate and/or kill target bacteria within the microbiome by the CRISPR-Cas system, lytic activity, or a combination thereof. In some embodiments, the bacteriophages are used to modulate and/or kill target bacteria within the microbiome of a subject. In some embodiments, the bacteriophages are used to selectively modulate and/or kill one or more target bacteria from a plurality of bacteria within the microbiome of a subject. In some embodiments, the bacteriophages are used to selectively modulate and/or kill one or more target entereopathogenic bacteria from a plurality of bacteria within the microbiome of a subject. In some embodiments, the target entereopathogenic bacterium is entereopathogenic E. Coli (EPEC). In some embodiments, the bacteriophages are used to selectively modulate and/or kill one or more target diarreagenic bacteria from a plurality of bacteria within the microbiome of a subject. In some embodiments, the target diarreagenic bacterium is diarreagenic E. coli (DEC). In some embodiments, the bacteriophages are used to selectively modulate and/or kill one or more target Shiga-toxin producing bacteria from a plurality of bacteria within the microbiome of a subject. In some embodiments, the target Shiga-toxin producing bacterium is Shiga-toxin producing E. coli (STEC).

In some embodiments, the bacteriophages are used to selectively modulate and/or kill one or more target enteropathogenic C. difficile bacteria strains within the microbiome of a subject including: CD043, CD05, CD073, CD093, CD180, CD106, CD128, CD199, CD111, CD108, CD25, CD148, CD154, FOBT195, CD03, CD038, CD112, CD196, CD105, UK1, UK6, BI-9, CD041, CD042, CD046, CD19, or 820291.

In some embodiments, the bacteriophages are used to modulate or kill target single or plurality of bacteria within the microbiome or gut flora of the gastrointestinal tract of a subject. Modification (e.g., dysbiosis) of the microbiome or gut flora increases the risk for health conditions such as diabetes, mental disorders, ulcerative colitis, colorectal cancer, autoimmune disorders, obesity, diabetes, diseases of the central nervous system and inflammatory bowel disease. An exemplary list of the bacteria associated with diseases and conditions of gastrointestinal tract and are being modulated or killed by the bacteriophages include strains, sub-strains, and enterotypes of enterobacteriaceae, pasteurellaceae, fusobacteriaceae, neisseriaceae, veillonellaceae, gemellaceae, bacteriodales, clostridiales, erysipelotrichaceae, bifidobacteriaceae bacteroides, faecalibacterium, roseburia, blautia, ruminococcus, coprococcus, streptococcus, dorea, blautia, ruminococcus, lactobacillus, enterococcus, streptococcus, Escherichia coli, Fusobacterium nucleatum, haemophilus parainfluenzae (pasteurellaceae), veillonella parvula, eikenella corrodens (neisseriaceae), gemella moribillum, bacteroides vulgatus, bacteroides caccae, Bifidobacterium bifidum, Bifidobacterium longum, bifidobacterium adolescentis, bifidobacterium dentum, blautia hansenii, ruminococcus gnavus, clostridium nexile, faecalibacterium prausnitzii, ruminoccus torques, clostridium bolteae, eubacterium rectale, roseburia intestinalis, coprococcus comes, actinomyces, lactococcus, roseburia, streptococcus, blautia, dialister, desulfovibrio, escherichia, lactobacillus, coprococcus, clostridium, bifidobacterium, klebsiella, granulicatella, eubacterium, anaerostipes, parabacteroides, coprobacillus, gordonibacter, collinsella, bacteroides, faecalibacterium, anaerotruncus, alistipes, haemophilus, anaerococcus, veillonella, arevotella, akkermansia, bilophila, sutterella, eggerthella, holdemania, gemella, peptoniphilus, rothia, enterococcus, pediococcus, citrobacter, odoribacter, enterobacteria, fusobacterium, and proteus.

In some embodiments, a bacteriophage disclosed herein is administered to a subject to promote a healthy microbiome. In some embodiments, a bacteriophage disclosed herein is administered to a subject to restore a subject's microbiome to a microbiome composition that promotes health. In some embodiments, a composition comprising a bacteriophage disclosed herein comprises a prebiotic or a third agent. In some embodiment, microbiome related disease or disorder is treated by a bacteriophage disclosed herein.

Environmental Therapy

In some embodiments, bacteriophages disclosed herein are further used for food and agriculture sanitation (including meats, fruits and vegetable sanitation), hospital sanitation, home sanitation, vehicle and equipment sanitation, industrial sanitation, etc. In some embodiments, bacteriophages disclosed herein are used for the removal of antibiotic-resistant or other undesirable pathogens from medical, veterinary, animal husbandry, or any additional environments bacteria are passed to humans or animals.

Environmental applications of phage in health care institutions are for equipment such as endoscopes and environments such as ICUs which are potential sources of nosocomial infection due to pathogens that are difficult or impossible to disinfect. In some embodiments, a phage disclosed herein is used to treat equipment or environments inhabited by bacterial genera such as Pseudomonas which become resistant to commonly used disinfectants. In some embodiments, phage compositions disclosed herein are used to disinfect inanimate objects. In some embodiments, an environment disclosed herein is sprayed, painted, or poured onto with aqueous solutions with phage titers. In some embodiment a solution described herein comprises between 10¹-10²⁰ plaque forming units (PFU)/ml. In some embodiments, a bacteriophage disclosed herein is applied by aerosolizing agents that include dry dispersants to facilitate distribution of the bacteriophage into the environment. In some embodiments, objects are immersed in a solution containing bacteriophage disclosed herein.

Sanitation

In some embodiments, bacteriophages disclosed herein are used as sanitation agents in a variety of fields. Although the terms “phage” or “bacteriophage” may be used, it should be noted that, where appropriate, this term should be broadly construed to include a single bacteriophage, multiple bacteriophages, such as a bacteriophage mixtures and mixtures of a bacteriophage with an agent, such as a disinfectant, a detergent, a surfactant, water, etc.

In some embodiments, bacteriophages are used to sanitize hospital facilities, including operating rooms, patient rooms, waiting rooms, lab rooms, or other miscellaneous hospital equipment. In some embodiments, this equipment includes electrocardiographs, respirators, cardiovascular assist devices, intraaortic balloon pumps, infusion devices, other patient care devices, televisions, monitors, remote controls, telephones, beds, etc. In some situations, the bacteriophage is applied through an aerosol canister. In some embodiments, bacteriophage is applied by wiping the phage on the object with a transfer vehicle.

In some embodiments, a bacteriophage described herein is used in conjunction with patient care devices. In some embodiment, bacteriophage is used in conjunction with a conventional ventilator or respiratory therapy device to clean the internal and external surfaces between patients. Examples of ventilators include devices to support ventilation during surgery, devices to support ventilation of incapacitated patients, and similar equipment. In some embodiments, the conventional therapy includes automatic or motorized devices, or manual bag-type devices such as are commonly found in emergency rooms and ambulances. In some embodiments, respiratory therapy includes inhalers to introduce medications such as bronchodilators as commonly used with chronic obstructive pulmonary disease or asthma, or devices to maintain airway patency such as continuous positive airway pressure devices.

In some embodiment, a bacteriophage described herein is used to cleanse surfaces and treat colonized people in an area where highly-contagious bacterial diseases, such as meningitis or enteric infections are present.

In some embodiments, water supplies are treated with a composition disclosed herein. In some embodiments, bacteriophage disclosed herein is used to treat contaminated water, water found in cisterns, wells, reservoirs, holding tanks, aqueducts, conduits, and similar water distribution devices. In some embodiments, the bacteriophage is applied to industrial holding tanks where water, oil, cooling fluids, and other liquids accumulate in collection pools. In some embodiments, a bacteriophage disclosed herein is periodically introduced to the industrial holding tanks in order to reduce bacterial growth.

In some embodiments, bacteriophages disclosed herein are used to sanitize a living area, such as a house, apartment, condominium, dormitory, or any living area. In some embodiments, the bacteriophage is used to sanitize public areas, such as theaters, concert halls, museums, train stations, airports, pet areas, such as pet beds, or litter boxes. In this capacity, the bacteriophage is dispensed from conventional devices, including pump sprayers, aerosol containers, squirt bottles, pre-moistened towelettes, etc, applied directly to (e.g., sprayed onto) the area to be sanitized, or be transferred to the area via a transfer vehicle, such as a towel, sponge, etc. In some embodiments, a phage disclosed herein is applied to various rooms of a house, including the kitchen, bedrooms, bathrooms, garage, basement, etc. In some embodiments, a phage disclosed herein is in the same manner as conventional cleaners. In some embodiments, the phage is applied in conjunction with (before, after, or simultaneously with) conventional cleaners provided that the conventional cleaner is formulated so as to preserve adequate bacteriophage biologic activity.

In some embodiments, a bacteriophage disclosed herein is added to a component of paper products, either during processing or after completion of processing of the paper products. Paper products to which a bacteriophage disclosed herein is added include, but are not limited to, paper towels, toilet paper, moist paper wipes.

Food Safety

In some embodiments, a bacteriophage described herein is used in any food product or nutritional supplement, for preventing contamination. Examples for food or pharmaceuticals products are milk, yoghurt, curd, cheese, fermented milks, milk based fermented products, ice-creams, fermented cereal based products, milk based powders, infant formulae or tablets, liquid suspensions, dried oral supplement, wet oral supplement, or dry-tube-feeding.

The broad concept of bacteriophage sanitation is applicable to other agricultural applications and organisms. Produce, including fruits and vegetables, dairy products, and other agricultural products. For example, freshly-cut produce frequently arrive at the processing plant contaminated with pathogenic bacteria. This has led to outbreaks of food-borne illness traceable to produce. In some embodiments, the application of bacteriophage preparations to agricultural produce substantially reduce or eliminate the possibility of food-borne illness through application of a single phage or phage mixture with specificity toward species of bacteria associated with food-borne illness. In some embodiments, bacteriophages are applied at various stages of production and processing to reduce bacterial contamination at that point or to protect against contamination at subsequent points.

In some embodiments, specific bacteriophages are applied to produce in restaurants, grocery stores, produce distribution centers. In some embodiments, bacteriophages disclosed herein are periodically or continuously applied to the fruit and vegetable contents of a salad bar. In some embodiments, the application of bacteriophages to a salad bar or to sanitize the exterior of a food item is a misting or spraying process or a washing process.

In some embodiments, a bacteriophage described herein is used in matrices or support media containing with packaging containing meat, produce, cut fruits and vegetables, and other foodstuffs. In some embodiments, polymers that are suitable for packaging are impregnated with a bacteriophage preparation.

In some embodiments, a bacteriophage described herein is used in farm houses and livestock feed. In some embodiments, on a farm raising livestock, the livestock is provided with bacteriophage in their drinking water, food, or both. In some embodiments, a bacteriophage described herein is sprayed onto the carcasses and used to disinfect the slaughter area.

The use of specific bacteriophages as biocontrol agents on produce provides many advantages. For example, bacteriophages are natural, non-toxic products that will not disturb the ecological balance of the natural microflora in the way the common chemical sanitizers do, but will specifically lyse the targeted food-borne pathogens. Because bacteriophages, unlike chemical sanitizers, are natural products that evolve along with their host bacteria, new phages that are active against recently emerged, resistant bacteria are rapidly identified when required, whereas identification of a new effective sanitizer is a much longer process, several years.

### Pharmaceutical Compositions

In some embodiments, the disclosure provides pharmaceutical compositions and methods of administering the same to treat bacterial, archaeal infections or to disinfect an area. In some embodiments, the pharmaceutical composition comprises any of the reagents discussed above in a pharmaceutically acceptable carrier. In some embodiments, a pharmaceutical composition or method disclosed herein treats Lung infections (CFP, NCFB, HAP/VAP) systemic infections (bacteremia, SSSI) GI microbiome dysbiosis (CDI) and/or urinary tract infections (cUTI).

In some embodiments, compositions disclosed herein comprise medicinal agents, pharmaceutical agents, carriers, adjuvants, dispersing agents, diluents, and the like.

In some embodiments, the bacteriophages disclosed herein are formulated for administration in a pharmaceutical carrier in accordance with suitable methods. In some embodiments, the manufacture of a pharmaceutical composition according to the disclosure, the bacteriophage is admixed with, inter alia, an acceptable carrier. In some embodiments, the carrier is a solid (including a powder) or a liquid, or both, and is preferably formulated as a unit-dose composition. In some embodiments, one or more bacteriophages are incorporated in the compositions disclosed herein, which are prepared by any suitable method of a pharmacy.

In some embodiment, a method of treating subject's in-vivo, comprising administering to a subject a pharmaceutical composition comprising a bacteriophage disclosed herein in a pharmaceutically acceptable carrier, wherein the pharmaceutical composition is administered in a therapeutically effective amount. In some embodiments, the administration of the bacteriophage to a human subject or an animal in need thereof are by any means known in the art.

In some embodiments, bacteriophages disclosed herein are for oral administration. In some embodiments, the bacteriophages are administered in solid dosage forms, such as capsules, tablets, and powders, or in liquid dosage forms, such as elixirs, syrups, and suspensions. In some embodiments, compositions and methods suitable for buccal (sub-lingual) administration include lozenges comprising the bacteriophages in a flavored base, usually sucrose and acacia or tragacanth; and pastilles comprising the bacteriophages in an inert base such as gelatin and glycerin or sucrose and acacia.

In some embodiments, methods and compositions of the present disclosure are suitable for parenteral administration comprising sterile aqueous and non-aqueous injection solutions of the bacteriophage. In some embodiments, these preparations are isotonic with the blood of the intended recipient. In some embodiments, these preparations comprise antioxidants, buffers, bacteriostals and solutes which render the composition isotonic with the blood of the intended recipient. In some embodiments, aqueous and non-aqueous sterile suspensions include suspending agents and thickening agents. In some embodiments, compositions disclosed herein are presented in unit\dose or multi-dose containers, for example sealed ampoules and vials, and are stored in a freeze-dried(lyophilized) condition requiring only the addition of the sterile liquid carrier, for example, saline or water for injection on immediately prior to use.

In some embodiment, methods and compositions suitable for rectal administration are presented as unit dose suppositories. In some embodiments, these are prepared by admixing the bacteriophage with one or more conventional solid carriers, for example, cocoa butter, and then shaping the resulting mixture. In some embodiments, methods and compositions suitable for topical application to the skin are in the form of an ointment, cream, lotion, paste, gel, spray, aerosol, or oil. In some embodiments, carriers which are used include petroleum jelly, lanoline, polyethylene glycols, alcohols, transdermal enhancers, and combinations of two or more thereof.

In some embodiments, methods and compositions suitable for transdermal administration are presented as discrete patches adapted to remain in intimate contact with the epidermis of the recipient for a prolonged period of time.

In some embodiments, methods and compositions suitable for nasal administration or otherwise administered to the lungs of a subject include any suitable means, e.g., administered by an aerosol suspension of respirable particles comprising the bacteriophage compositions, which the subject inhales. In some embodiments, the respirable particles are liquid or solid. As used herein, “aerosol” includes any gas-borne suspended phase, which is capable of being inhaled into the bronchioles or nasal passages. In some embodiments, aerosols of liquid particles are produced by any suitable means, such as with a pressure-driven aerosol nebulizer or an ultrasonic nebulizer. In some embodiments, aerosols of solid particles comprising the composition is produced with any solid particulate medicament aerosol generator, by techniques known in the pharmaceutical art.

In some embodiment, methods and compositions suitable for administering bacteriophages disclosed herein to a surface of an object or subject includes aqueous solutions. In some embodiments, such aqueous solutions are sprayed onto the surface of an object or subject. In some embodiment, the aqueous solutions are used to irrigate and clean a physical wound of a subject form foreign debris including bacteria.

In some embodiments, the bacteriophages disclosed herein are administered to the subject in a therapeutically effective amount. In some embodiments, at least one bacteriophage composition disclosed herein is formulated as a pharmaceutical formulation. In some embodiments, a pharmaceutical formulation comprises 1, 2, 3, 4, 5, 6, 7, 8, 9, 10, 11, 12, 13, 14, 15, 16, 17, 18, 19, 20 or more bacteriophage disclosed herein. In some instances, a pharmaceutical formulation comprises a bacteriophage described herein and at least one of: an excipient, a diluent, or a carrier.

In some embodiments, a pharmaceutical formulation comprises an excipient. Excipients are described in the Handbook of Pharmaceutical Excipients, American Pharmaceutical Association (1986) and includes but are not limited to solvents, dispersion media, diluents, or other liquid vehicles, dispersion or suspension aids, surface active agents, isotonic agents, thickening or emulsifying agents, preservatives, solid binders, and lubricants.

Non-limiting examples of suitable excipients include but is not limited to a buffering agent, a preservative, a stabilizer, a binder, a compaction agent, a lubricant, a chelator, a dispersion enhancer, a disintegration agent, a flavoring agent, a sweetener, a coloring agent.

In some embodiments, an excipient is a buffering agent. Non-limiting examples of suitable buffering agents include but is not limited to sodium citrate, magnesium carbonate, magnesium bicarbonate, calcium carbonate, and calcium bicarbonate. In some embodiments, a pharmaceutical formulation comprises any one or more buffering agent listed: sodium bicarbonate, potassium bicarbonate, magnesium hydroxide, magnesium lactate, magnesium glucomate, aluminum hydroxide, sodium citrate, sodium tartrate, sodium acetate, sodium carbonate, sodium polyphosphate, potassium polyphosphate, sodium pyrophosphate, potassium pyrophosphate, disodium hydrogen phosphate, dipotassium hydrogen phosphate, trisodium phosphate, tripotassium phosphate, potassium metaphosphate, magnesium oxide, magnesium hydroxide, magnesium carbonate, magnesium silicate, calcium acetate, calcium glycerophosphate, calcium chloride, calcium hydroxide and other calcium salts.

In some embodiments an excipient is a preservative. Non-limiting examples of suitable preservatives include but is not limited to antioxidants, such as alpha-tocopherol and ascorbate, and antimicrobials, such as parabens, chlorobutanol, and phenol. In some embodiments, antioxidants include but not limited to EDTA, citric acid, ascorbic acid, butylated hydroxytoluene (BHT), butylated hydroxy anisole (BHA), sodium sulfite, p-amino benzoic acid, glutathione, propyl gallate, cysteine, methionine, ethanol and N-acetyl cysteine. In some embodiments, preservatives include validamycin A, TL-3, sodium ortho vanadate, sodium fluoride, N-a-tosyl-Phe-chloromethylketone, N-a-tosyl-Lys-chloromethylketone, aprotinin, phenylmethylsulfonyl fluoride, diisopropylfluorophosphate, protease inhibitor, reducing agent, alkylating agent, antimicrobial agent, oxidase inhibitor, or other inhibitor.

In some embodiments, a pharmaceutical formulation comprises a binder as an excipient. Non-limiting examples of suitable binders include starches, pregelatinized starches, gelatin, polyvinylpyrolidone, cellulose, methylcellulose, sodium carboxymethylcellulose, ethylcellulose, polyacrylamides, polyvinyloxoazolidone, polyvinylalcohols, C₁₂-C₁₈ fatty acid alcohol, polyethylene glycol, polyols, saccharides, oligosaccharides, and combinations thereof.

In some embodiments, the binders that are used in a pharmaceutical formulation are selected from starches such as potato starch, corn starch, wheat starch; sugars such as sucrose, glucose, dextrose, lactose, maltodextrin; natural and synthetic gums; gelatine; cellulose derivatives such as microcrystalline cellulose, hydroxypropyl cellulose, hydroxyethyl cellulose, hydroxypropyl methyl cellulose, carboxymethyl cellulose, methyl cellulose, ethyl cellulose; polyvinylpyrrolidone (povidone); polyethylene glycol (PEG); waxes; calcium carbonate; calcium phosphate; alcohols such as sorbitol, xylitol, mannitol and water or a combination thereof.

In some embodiments, a pharmaceutical formulation comprises a lubricant as an excipient. Non-limiting examples of suitable lubricants include magnesium stearate, calcium stearate, zinc stearate, hydrogenated vegetable oils, sterotex, polyoxyethylene monostearate, talc, polyethylene glycol, sodium benzoate, sodium lauryl sulfate, magnesium lauryl sulfate, and light mineral oil. In some embodiments, lubricants that are in a pharmaceutical formulation are selected from metallic stearates (such as magnesium stearate, calcium stearate, aluminum stearate), fatty acid esters (such as sodium stearyl fumarate), fatty acids (such as stearic acid), fatty alcohols, glyceryl behenate, mineral oil, paraffins, hydrogenated vegetable oils, leucine, polyethylene glycols (PEG), metallic lauryl sulphates (such as sodium lauryl sulphate, magnesium lauryl sulphate), sodium chloride, sodium benzoate, sodium acetate and talc or a combination thereof.

In some embodiments, an excipient comprises a flavoring agent. In some embodiments, flavoring agents includes natural oils; extracts from plants, leaves, flowers, and fruits; and combinations thereof.

In some embodiments, an excipient comprises a sweetener. Non-limiting examples of suitable sweeteners include glucose (corn syrup), dextrose, invert sugar, fructose, and mixtures thereof (when not used as a carrier); saccharin and its various salts such as a sodium salt; dipeptide sweeteners such as aspartame; dihydrochalcone compounds, glycyrrhizin; Stevia Rebaudiana (Stevioside); chloro derivatives of sucrose such as sucralose; and sugar alcohols such as sorbitol, mannitol, sylitol, and the like.

In some instances, a pharmaceutical formulation comprises a coloring agent. Non-limiting examples of suitable color agents include food, drug and cosmetic colors (FD&C), drug and cosmetic colors (D&C), and external drug and cosmetic colors (Ext. D&C).

In some embodiments, the pharmaceutical formulation disclosed herein comprises a chelator. In some embodiments, a chelator includes ethylenediamine-N,N,N′,N′-tetraacetic acid (EDTA); a disodium, trisodium, tetrasodium, dipotassium, tripotassium, dilithium and diammonium salt of EDTA; a barium, calcium, cobalt, copper, dysprosium, europium, iron, indium, lanthanum, magnesium, manganese, nickel, samarium, strontium, or zinc chelate of EDTA.

In some instances, a pharmaceutical formulation comprises a diluent. Non-limiting examples of diluents include water, glycerol, methanol, ethanol, and other similar biocompatible diluents. In some embodiments, a diluent is an aqueous acid such as acetic acid, citric acid, maleic acid, hydrochloric acid, phosphoric acid, nitric acid, sulfuric acid, or similar.

In some embodiments, a pharmaceutical formulation comprises a surfactant. In some embodiments, surfactants are be selected from, but not limited to, polyoxyethylene sorbitan fatty acid esters (polysorbates), sodium lauryl sulphate, sodium stearyl fumarate, polyoxyethylene alkyl ethers, sorbitan fatty acid esters, polyethylene glycols (PEG), polyoxyethylene castor oil derivatives, docusate sodium, quaternary ammonium compounds, aminoacids such as L-leucine, sugar esters of fatty acids, glycerides of fatty acids or a combination thereof.

In some instances, a pharmaceutical formulation comprises an additional pharmaceutical agent. In some embodiments, an additional pharmaceutical agent is an antibiotic agent. In some embodiments, an antibiotic agent is of the group consisting of aminoglycosides, ansamycins, carbacephem, carbapenems, cephalosporins (including first, second, third, fourth and fifth generation cephalosporins), lincosamides, macrolides, monobactams, nitrofurans, quinolones, penicillin, sulfonamides, polypeptides or tetracycline.

In some embodiments, an antibiotic agent described herein is an aminoglycoside such as Amikacin, Gentamicin, Kanamycin, Neomycin, Netilmicin, Tobramycin or Paromomycin. In some embodiments, an antibiotic agent described herein is an Ansamycin such as Geldanamycin or Herbimycin

In some embodiments, an antibiotic agent described herein is a carbacephem such as Loracarbef. In some embodiments, an antibiotic agent described herein is a carbapenem such as Ertapenem, Doripenem, Imipenem/Cilastatin or Meropenem.

In some embodiments, an antibiotic agent described herein is a cephalosporins (first generation) such as Cefadroxil, Cefazolin, Cefalexin, Cefalotin or Cefalothin, or alternatively a Cephalosporins (second generation) such as Cefaclor, Cefamandole, Cefoxitin, Cefprozil or Cefuroxime. In some embodiments, an antibiotic agent is a Cephalosporins (third generation) such as Cefixime, Cefdinir, Cefditoren, Cefoperazone, Cefotaxime, Cefpodoxime, Ceftibuten, Ceftizoxime and Ceftriaxone or a Cephalosporins (fourth generation) such as Cefepime or Ceftobiprole.

In some embodiments, an antibiotic agent described herein is a lincosamide such as Clindamycin and Azithromycin, or a macrolide such as Azithromycin, Clarithromycin, Dirithromycin, Erythromycin, Roxithromycin, Troleandomycin, Telithromycin and Spectinomycin.

In some embodiments, an antibiotic agent described herein is a monobactams such as Aztreonam, or a nitrofuran such as Furazolidone or Nitrofurantoin.

In some embodiments, an antibiotic agent described herein is a penicillin such as Amoxicillin, Ampicillin, Azlocillin, Carbenicillin, Cloxacillin, Dicloxacillin, Flucloxacillin, Mezlocillin, Nafcillin, Oxacillin, Penicillin G or V, Piperacillin, Temocillin and Ticarcillin.

In some embodiments, an antibiotic agent described herein is a sulfonamide such as Mafenide, Sulfonamidochrysoidine, Sulfacetamide, Sulfadiazine, Silver sulfadiazine, Sulfamethizole, Sulfamethoxazole, Sulfanilimide, Sulfasalazine, Sulfisoxazole, Trimethoprim, or Trimethoprim-Sulfamethoxazole (Co-trimoxazole) (TMP-SMX).

In some embodiments, an antibiotic agent described herein is a quinolone such as Ciprofloxacin, Enoxacin, Gatifloxacin, Levofloxacin, Lomefloxacin, Moxifloxacin, Nalidixic acid, Norfloxacin, Ofloxacin, Trovafloxacin, Grepafloxacin, Sparfloxacin and Temafloxacin.

In some embodiments, an antibiotic agent described herein is a polypeptide such as Bacitracin, Colistin or Polymyxin B.

In some embodiments, an antibiotic agent described herein is a tetracycline such as Demeclocycline, Doxycycline, Minocycline or Oxytetracycline.

Dose

Dose and duration of the administration of a composition disclosed herein will depend on a variety of factors, including the subject's age, subject's weight, and tolerance of the phage. In some embodiments, a bacteriophage disclosed herein is administered to patients by oral administration. In some embodiments, a dose of phage between 10³ and 10²⁰ PFU is given. For example, in some embodiments, the bacteriophage is present in a composition in an amount between 10³ and 10¹¹ PFU. In some embodiments, the bacteriophage is present in a composition in an amount about 10³, 10⁴, 10⁵, 10⁶, 10⁷, 10⁸, 10⁹, 10¹⁰, 10¹¹, 10¹², 10¹³, 10¹⁴, 10¹⁵, 10¹⁶, 10¹⁷, 10¹⁸, 10¹⁹, 10²⁰, 10²¹, 10²², 10²³, 10²⁴ PFU or more. In some embodiments, the bacteriophage is present in a composition in an amount of less than 10¹ PFU. In some embodiments, the bacteriophage is present in a composition in an amount between 10¹ and 10⁸, 10⁴ and 10⁹, 10⁵ and 10¹⁰, or 10⁷ and 10¹¹ PFU. In some embodiments, a bacteriophage or a mixture is administered to a subject in need thereof 1, 2, 3, 4, 5, 6, 7, 8, 9, 10, 11, 12, 13, 14, 15, 16, 17, 18, 19, 20, 21, 22, 23, or 24 times a day. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, or 21 times a week. In some embodiments, a bacteriophage or a mixture is administered to a subject in need thereof at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month.

In some embodiments, the compositions (bacteriophage) disclosed herein are administered before, during, or after the occurrence of a disease or condition. In some embodiment, the timing of administering the composition containing the bacteriophage varies. In some embodiments, the pharmaceutical compositions are used as a prophylactic and are administered continuously to subjects with a propensity to conditions or diseases in order to prevent the occurrence of the disease or condition. In some embodiments, pharmaceutical compositions are administered to a subject during or as soon as possible after the onset of the symptoms. In some embodiments, the administration of the compositions is initiated within the first 48 hours of the onset of the symptoms, within the first 24 hours of the onset of the symptoms, within the first 6 hours of the onset of the symptoms, or within 3 hours of the onset of the symptoms. In some embodiments, the initial administration of the composition is via any route practical, such as by any route described herein using any formulation described herein. In some embodiments, the compositions is administered as soon as is practicable after the onset of a disease or condition is detected or suspected, and for a length of time necessary for the treatment of the disease, such as, for example, from about 1 month to about 3 months. In some embodiments, the length of treatment will vary for each subject.

### Kits

Disclosed herein are kits for use. In some embodiments, the kit comprises the nucleic acid constructs for the CRISPR arrays, transcriptional activators, and/or anti-CRISPR polypeptides, as well as the bacteriophages and/or any other vectors/expression cassettes disclosed herein in a form suitable for introduction into a cell and/or administration to a subject. In some embodiments, the kit comprises other therapeutic agents, carriers, buffers, containers, devices for administration, and the like. In some embodiments, the kit comprises labels and/or instructions for repression of expression a target gene and/or modulation of repression of expression of a target gene. In some embodiments, labeling and/or instructions includes, for example, information concerning the amount, frequency and method of introduction and/or administration of the nucleic acid constructs for the CRISPR arrays, transcriptional activators, and anti-CRISPR polypeptides, as well as the bacteriophages and/or any other vectors/expression cassettes.

In some embodiments, a kit for the killing of one target bacterium is provided, said kit comprising, consisting essentially of, consisting of nucleic acid constructs for the CRISPR arrays, transcriptional activators, and/or anti-CRISPR polypeptides, as well as the bacteriophages and/or any other vectors/expression cassettes necessary to achieve killing of the target bacteria by any embodiment disclosed herein.

In some embodiments, a kit is provided for modulating the activity of a CRISPR-Cas system in a target bacterium is provided, the kit comprising, consisting essentially of, consisting of nucleic acid constructs for the CRISPR arrays, transcriptional activators, and anti-CRISPR polypeptides, as well as the bacteriophages and/or any other vectors/expression cassettes necessary to achieve modulation of a CRISPR-Cas system in a target bacteria by any embodiment disclosed herein.

In some embodiments, the nucleic acid constructs for the CRISPR arrays, transcriptional activators, and/or anti-CRISPR polypeptides of said kits are comprised on a single vector or expression cassette or on separate vectors or expression cassettes or within a single bacteriophage or a plurality of bacteriophages. In some embodiments, a kit comprises one or more bacteriophage disclosed herein. In some embodiments, the kits comprise instructions for use. In some embodiments, the instructions for practicing the methods are recorded on a suitable recording medium. In some embodiments, the instructions are printed on a substrate, such as paper or plastic, etc. In some embodiments, the instructions are present in the kits as a package insert, in the labeling of the container of the kit or components thereof (i.e., associated with the packaging or subpackaging) etc. In some embodiments, the instructions are present as an electronic storage data file present on a suitable computer readable storage medium, e.g. CD-ROM, diskette, flash drive, etc. In some embodiments, the actual instructions are not present in the kit, but means for obtaining the instructions from a remote source (e.g. via the Internet), are provided. In some embodiments, the kit includes a web address where the instructions are viewed and/or from which the instructions are downloaded.

Certain embodiments disclosed herein, both in their methods and compositions, will now are described with reference to the following examples. It should be appreciated that these examples are not intended to limit the scope of the claims to the disclosure, but are rather intended to be exemplary of certain embodiments. Any variations in the exemplified methods that occur to the skilled artisan are intended to fall within the scope of the disclosure.

### EXAMPLES

### Example 1: Overview for Generating CRISPR Enhanced Bacteriophages

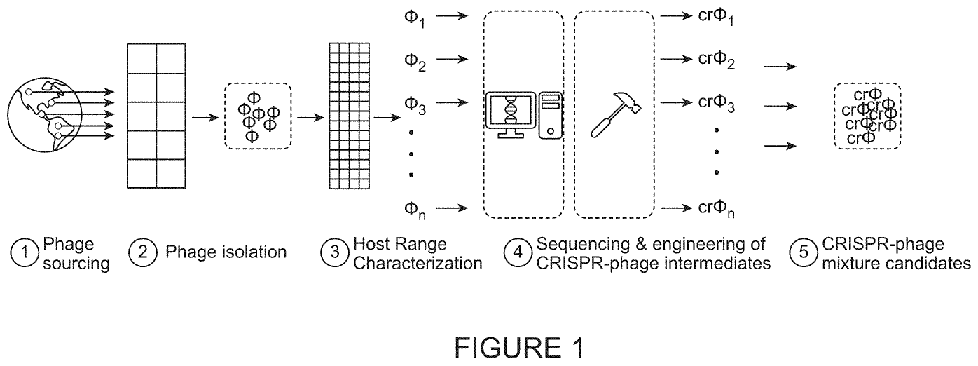

CRISPR-enhanced bacteriophages are phages that have been engineered to express CRISPR RNA constructs from a bacteriophage genome that maintains the essential genes for lytic lifestyle. The steps involved are sourcing, isolating and identifying bacteriophages and cocktails of bacteriophages with broad host ranges against bacteria followed by engineering each phage to carry an expression construct (for example, crRNA) that targets the bacterium's genome and validating optimized combinations of crPhages to be used as a clinical lead candidate. In some embodiments, the general processes are as schematically shown in steps 1-5 of FIG. 1. Steps 1-5 are designed to identify a suitable number of wild-type bacteriophages such that they:

1. Meet minimum quality standards (absence of lysogeny, virulence genes or antibiotic resistance genes) proposed in Table 1 below:

TABLE 1
Summary of phage characterizations

| Test/characteristic | Method |
|---|---|
| Genome size (kb) | Genome sequencing |
| Family of Caudovirales | Transmission electron microscopy |
| Host range activity | Host range analysis against uropathogenic |
| | E. coli clinical isolates and representative |
| | E. coli strains |
| Genome sequence | Genome sequencing |
| DNA restriction profile | Restriction enzyme digestion/ |
| | electrophoresis |
| Typing | PCR specific to engineered insert |
| Lifestyle (lytic, temperate) | DNA analysis |
| Absence of generalized | Microbiological transduction assay |
| transduction | |
| Absence of virulence genes | Genome sequence analysis |
| Absence of antibiotic | Genome sequence analysis |
| resistance genes | |

2. Have collective activity against approximately 90% or greater of the clinical isolate panel.

3. Result in infection of each strain by at least 2 phages within the cocktail (mixture of two or more phages), intended to ensure strain sensitivity to the cocktail in the event of resistance to any single bacteriophage. In some embodiments, a cocktail described herein in comprises at least 2, 3, 4, 5, 6, 7, 8, 9, 10, 20, 30, 50, 100 or more bacteriophages.

4. Include genetic engineering of each candidate bacteriophage to express a crRNA construct from the wild-type genome. Each engineered crPhage is intended to retain lytic activity. crPhages will then be subjected to in vitro analyses to assess host range and in vitro efficacy. These studies are intended to confirm that crPhages retain broad host range, if not expanded host range by ability to transduce lethal crRNA constructs in the absence of productive lytic infection, and improved lethality for each crPhage to the cognate wild-type bacteriophage.

5. Identifying crPhages for use in a cocktail that is optimized to improve host range, manufacturing limitations or nonclinical efficacy.

### Example 2: Phage Isolation

Bacteriophage vectors were obtained from environmental sources in North Carolina. Phages were directly isolated from these samples by co-incubating with uropathgenic E. coli s ECOR 14, 62, 64 and 71. Each phage was then subjected to clonal purification by single plaque isolation across a total of three rounds of double agar overlays prior to amplification, filtration and long-term storage at 4 degrees Celsius. Each phage was amplified, filtered, subjected to cesium chloride gradient purification and dialyzed into 1× tris-buffered saline. Phage genomic DNA was extracted from purified phage stocks using a column-based phage DNA preparation kit (Norgen) and submitted for genome sequencing by MiSeq or PacBio sequencing, as appropriate, and assembly at a third party vendor (Genewiz). A previously isolated phage, K1F was obtained that has been previously shown to infect uropathogenic E. coli isolates and have known genomes (K1F: NC_007456).

### Example 3: Phage Host Range Analysis

This library of wild-type bacteriophages was individually characterized against an E. coli panel for the ability to replicate and lyse each target. This process was expected to result in a wild-type bacteriophage panel that lyses approximately 90% of a panel of uropathogenic E. coli and displays minimal resistance during 24-hour challenge studies. From this master library, an abbreviated wild-type bacteriophage library was generated with retained predicted activity against approximately 90% of the uropathogenic E. coli panel.

Each phage was tested for lytic activity against an evolutionarily broad panel of E. coli. Briefly, phages were produced to high titer (10⁹-10¹¹ PFU/mL), filtered and left suspended in growth media. Each target host was grown to mid-log phase and incorporated in soft agar overlays to create bacterial lawns. Phages were serially diluted down to approximately 10³-10⁵ PFU/mL and 5 microliters of each dilution was spot plated on each bacterial lawn. Host sensitivity to each phage was defined as any observable zone of clearance within each spot. In some cases, this analysis includes some phages that adsorb to a target host and cause a phenomenon termed lysis-from-without rather than lysis-from-within caused by productive lytic infection. However, we intentionally chose not to exclude these data points under the assumption that lysis-from-without resulting from adsorption still permits DNA transduction even in the absence of productive lytic infection.

From this preliminary analysis, 10 phages were chosen that collectively infect 17 of 18 isolates tested (94%) in the panel, including 4 urinary pathogenic s isolated from human patients as shown in Table 2 below. Phages were isolated or tested against ECOR 14, 62, 64 or 71 as highlighted and then tested against the broader panel shown. Importantly, phage host range was considered as all productive lysis events and include events that resulted from lysis-from-without.

TABLE 2
Host range analysis of isolated bacteriophages against diverse set of E. coli stains.

| | | Phage | | | | | |
|---|---|---|---|---|---|---|---|
| | | ΦECOR71-2 | ΦECOR71-3 | ΦECOR71-4 | ΦECOR71-5 | ΦECOR71-6 | ΦECOR71-7 |
| E. coli | ECOR2 | 0 | 1 | 0 | 0 | 0 | 1 |
| Strain | ECOR5 | 0 | 1 | 0 | 1 | 1 | 1 |
| | ECOR14 | 1 | 0 | 0 | 0 | 0 | 0 |
| | (UTI Isolate) | | | | | | |
| | ECOR21 | 0 | 1 | 0 | 0 | 0 | 1 |
| | ECOR27 | 0 | 1 | 0 | 0 | 0 | 1 |
| | ECOR29 | 0 | 1 | 0 | 0 | 0 | 1 |
| | ECOR35 | 1 | 0 | 0 | 0 | 0 | 0 |
| | ECOR36 | 1 | 0 | 0 | 0 | 0 | 0 |
| | ECOR41 | 1 | 0 | 0 | 0 | 0 | 0 |
| | ECOR47 | 1 | 1 | 1 | 0 | 1 | 1 |
| | ECOR51 | 0 | 0 | 0 | 0 | 0 | 1 |
| | ECOR56 | 1 | 1 | 0 | 0 | 0 | 1 |
| | ECOR58 | 0 | 1 | 0 | 0 | 0 | 1 |
| | ECOR62 | 1 | 1 | 0 | 0 | 0 | 1 |
| | (UTI Isolate) | | | | | | |
| | ECOR64 | 0 | 1 | 1 | 1 | 1 | 1 |
| | (UTI Isolate) | | | | | | |
| | ECOR71 | 1 | 1 | 1 | 1 | 1 | 1 |
| | (UTI Isolate) | | | | | | |
| | EV36 | 1 | 1 | 0 | 0 | 0 | 1 |
| | CFT073 | 0 | 0 | 0 | 0 | 0 | 0 |
| | (UPEC Strain) | | | | | | |
| | # strains | 9/18 | 12/18 | 3/18 | 3/18 | 4/18 | 3/18 |
| | sensitive to phage | | | | | | |
| | % of strains | 50% | 67% | 17% | 17% | 22% | 72% |
| | sensitive to phage | | | | | | |

| | | | | | | # phages |
|---|---|---|---|---|---|---|
| | | Phage | | | | isolated |
| | | ΦECOR71-8 | ΦECOR71-10 | ΦECOR14-1 | ΦK1F | per strain |
| E. coli | ECOR2 | 1 | 0 | 0 | 0 | 3/10 |
| Strain | ECOR5 | 0 | 0 | 0 | 0 | 4/10 |
| | ECOR14 | 0 | 1 | 1 | 0 | 3/10 |
| | (UTI Isolate) | | | | | |
| | ECOR21 | 1 | 0 | 0 | 0 | 3/10 |
| | ECOR27 | 1 | 0 | 0 | 0 | 3/10 |
| | ECOR29 | 1 | 0 | 0 | 0 | 3/10 |
| | ECOR35 | 0 | 0 | 1 | 1 | 3/10 |
| | ECOR36 | 0 | 0 | 1 | 1 | 3/10 |
| | ECOR41 | 0 | 1 | 1 | 1 | 4/10 |
| | ECOR47 | 1 | 1 | 1 | 0 | 8/10 |
| | ECOR51 | 1 | 1 | 0 | 0 | 3/10 |
| | ECOR56 | 1 | 1 | 1 | 0 | 6/10 |
| | ECOR58 | 1 | 0 | 0 | 0 | 3/10 |
| | ECOR62 | 1 | 1 | 0 | 1 | 6/10 |
| | (UTI Isolate) | | | | | |
| | ECOR64 | 1 | 0 | 0 | 0 | 6/10 |
| | (UTI Isolate) | | | | | |
| | ECOR71 | 1 | 1 | 1 | 0 | 9/10 |
| | (UTI Isolate) | | | | | |
| | EV36 | 1 | 1 | 1 | 1 | 7/10 |
| | CFT073 | 0 | 0 | 0 | 0 | 0/10 |
| | (UPEC Strain) | | | | | |
| | # strains | 12/18 | 8/18 | 8/18 | 5/18 | |
| | sensitive to phage | | | | | |
| | % of strains | 67% | 44% | 44% | 28% | |
| | sensitive to phage | | | | | |

Bold text rows demonstrated preliminary host range coverage against uropathogenic E. coli isolates.

### Example 4: Primary Resistance to Isolate Phages

Using selected E. coli that are susceptible to multiple wild-type bacteriophages in this abbreviated library, individual bacteriophages were then assessed for ability to generate resistant clones in treatment-naïve hosts after overnight incubation with a challenge phage. Apparent resistant colonies to each individual wild-type bacteriophage were clonally isolated and re-challenged with the original bacteriophage and the others within the library. These data were used to inform which bacteriophages are likely to generate rapid primary resistance, defined as stable resistance phenotypes after re-challenge with the same bacteriophage. Stable resistance phenotypes did not exhibit obvious lysis by inspection of growth curves during 24-hour cultures in media comprising a challenge bacteriophage. Bacteriophages that generate primary resistance in >50% of clones were excluded from further study. A further characterization step then measures the sensitivity of each clone to the other wild-type bacteriophages in the abbreviated library.

All phage stocks were produced as crude lysates of the wild-type, wild-type bacteriophage in growth media by fermentation, filtration and validation of >10¹⁰ PFU/mL titers. To determine the empirical resistance profiles for each wild-type phage in the proposed 10-phage cocktail, putative resistant clones were generated for specific host: phage pairings by incubating ˜108 colony forming units of an E. coli host with a high-titer lysate (>10⁸ PFU, MOI>1.0) in a double agar overlay. After overnight culture, putative resistant clones were isolated by triple colony purification. After outgrowth, each clone was subjected to challenge with each original phage from the abbreviated 10-phage cocktail. Notably, numerous clones with apparent resistance phenotypes, defined as survival after initial challenge, apparently regained sensitivity after outgrowth in the absence of phage challenge as seen Table 3 below:

TABLE 3
Resistance profile data for selected crPhages against E. coli ECOR71.

| Phage: | | | | | | | | | | | | |
|---|---|---|---|---|---|---|---|---|---|---|---|---|
| host | | | | | | | | | | | | |
| clone | Rep | PlateID | 71-2 | 71-3 | 71-4 | 71-5 | 71-6 | 71-7 | 71-8 | 71-10 | 14-1 | KIF |
| 71-2 | 1 | ECOR71:φECOR71-2-R1 | R | S | R | R | R | S | S | S | R | S |
| 71-2 | 2 | ECOR71:φECOR71-2-R2 | R | S | R | R | R | S | S | S | S | S |
| 71-2 | 3 | ECOR71:φECOR71-2-R3 | R | S | R | R | R | S | S | S | S | T |
| 71-2 | 4 | ECOR71:φECOR71-2-R4 | R | S | R | R | R | S | S | S | S | S |
| 71-2 | 5 | ECOR71:φECOR71-2-R5 | R | S | R | R | R | S | S | S | S | S |
| 71-2 | 6 | ECOR71:φECOR71-2-R6 | R | S | S | S | S | S | S | S | S | S |
| 71-2 | 7 | ECOR71:φECOR71-2-R7 | R | S | R | R | R | S | S | S | S | S |
| 71-2 | 8 | ECOR71:φECOR71-2-R8 | R | S | R | R | R | S | S | R | R | T |
| 71-3 | 1 | ECOR71:φECOR71-3-R1 | R | S | S | R | R | S | S | S | S | T |
| 71-3 | 2 | ECOR71:φECOR71-3-R2 | R | S | S | S | S | S | S | S | S | S |
| 71-3 | 3 | ECOR71:φECOR71-3-R3 | R | S | S | R | R | S | S | S | S | S |
| 71-3 | 4 | ECOR71:φECOR71-3-R4 | R | S | S | R | S | S | R | R | R | R |
| 71-3 | 5 | ECOR71:φECOR71-3-R5 | R | S | S | R | R | S | R | S | S | S |
| 71-3 | 6 | ECOR71:φECOR71-3-R6 | R | S | S | S | S | S | S | S | S | S |
| 71-3 | 7 | ECOR71:φECOR71-3-R7 | R | S | S | R | R | S | S | R | S | S |
| 71-3 | 8 | ECOR71:φECOR71-3-R8 | R | S | S | R | R | S | S | S | S | S |
| 71-4 | 1 | ECOR71:φECOR71-4-R1 | S | S | S | S | S | S | S | S | S | S |
| 71-4 | 2 | ECOR71:φECOR71-4-R2 | S | S | S | S | S | S | S | S | S | S |
| 71-4 | 3 | ECOR71:φECOR71-4-R3 | S | S | S | S | S | S | S | S | S | S |
| 71-4 | 4 | ECOR71:φECOR71-4-R4 | S | S | S | S | S | S | R | S | S | S |
| 71-4 | 5 | ECOR71:φECOR71-4-R5 | S | S | S | S | S | S | S | S | S | S |
| 71-4 | 6 | ECOR71:φECOR71-4-R6 | S | S | S | S | S | S | S | S | S | S |
| 71-4 | 7 | ECOR71:φECOR71-4-R7 | S | S | S | S | S | S | S | S | S | S |
| 71-4 | 8 | ECOR71:φECOR71-4-R8 | S | S | S | S | S | S | S | S | S | S |
| 71-5 | 1 | ECOR71:φECOR71-5-R1 | S | S | S | S | S | S | S | S | S | S |
| 71-5 | 2 | ECOR71:φECOR71-5-R2 | S | S | S | S | S | S | S | S | S | S |
| 71-5 | 3 | ECOR71:φECOR71-5-R3 | S | S | S | S | S | S | S | S | S | S |
| 71-5 | 4 | ECOR71:φECOR71-5-R4 | S | S | S | S | S | S | T | S | S | S |
| 71-5 | 5 | ECOR71:φECOR71-5-R5 | S | S | S | S | S | S | S | S | S | S |
| 71-5 | 6 | ECOR71:φECOR71-5-R6 | S | S | S | S | S | S | S | S | S | S |
| 71-5 | 7 | ECOR71:φECOR71-5-R7 | S | S | S | S | S | S | R | S | S | S |
| 71-5 | 8 | ECOR71:φECOR71-5-R8 | S | S | S | S | S | S | R | S | S | S |
| 71-6 | 1 | ECOR71:φECOR71-6-R1 | S | S | S | S | S | S | S | S | S | S |
| 71-6 | 2 | ECOR71:φECOR71-6-R2 | S | S | S | S | S | S | S | S | S | S |
| 71-6 | 3 | ECOR71:φECOR71-6-R3 | S | S | S | S | R | S | S | S | S | S |
| 71-6 | 4 | ECOR71:φECOR71-6-R4 | S | S | S | S | S | S | R | S | S | S |
| 71-6 | 5 | ECOR71:φECOR71-6-R5 | S | S | S | S | S | S | S | S | R | S |
| 71-6 | 6 | ECOR71:φECOR71-6-R6 | S | S | S | S | S | S | S | S | S | S |
| 71-6 | 7 | ECOR71:φECOR71-6-R7 | S | S | S | S | S | S | S | S | S | S |
| 71-6 | 8 | ECOR71:φECOR71-6-R8 | S | S | S | S | S | S | S | S | S | S |
| 71-7 | 1 | ECOR71:φECOR71-7-R1 | R | X | X | X | X | X | X | S | X | X |
| 71-7 | 2 | ECOR71:φECOR71-7-R2 | R | S | S | R | R | S | S | S | S | S |
| 71-7 | 3 | ECOR71:φECOR71-7-R3 | R | S | S | R | R | S | S | S | S | S |
| 71-7 | 4 | ECOR71:φECOR71-7-R4 | R | S | R | R | R | S | S | S | S | S |
| 71-7 | 5 | ECOR71:φECOR71-7-R5 | R | S | S | R | R | S | R | R | S | S |
| 71-7 | 6 | ECOR71:φECOR71-7-R6 | R | S | R | R | R | S | S | S | S | S |
| 71-7 | 7 | ECOR71:φECOR71-7-R7 | R | S | R | R | R | S | S | S | S | S |
| 71-7 | 8 | ECOR71:φECOR71-7-R8 | R | R | S | R | R | S | R | R | R | S |
| 71-8 | 1 | ECOR71:φECOR71-8-R1 | R | S | R | X | R | X | X | X | X | X |
| 71-8 | 2 | ECOR71:φECOR71-8-R2 | R | S | R | R | R | S | T | S | S | S |
| 71-8 | 3 | ECOR71:φECOR71-8-R3 | R | S | X | R | X | R | X | X | X | X |
| 71-8 | 4 | ECOR71:φECOR71-8-R4 | R | S | R | R | R | S | S | S | T | S |
| 71-8 | 5 | ECOR71:φECOR71-8-R5 | R | R | S | R | R | S | R | R | S | S |
| 71-8 | 6 | ECOR71:φECOR71-8-R6 | R | S | R | R | R | S | S | S | S | S |
| 71-8 | 7 | ECOR71:φECOR71-8-R7 | R | S | S | R | R | S | S | S | S | S |
| 71-8 | 8 | ECOR71:φECOR71-8-R8 | R | S | S | R | R | S | R | R | R | S |
| 71-10 | 1 | ECOR71:φECOR71-10-R1 | R | S | S | S | S | S | S | S | X | S |
| 71-10 | 2 | ECOR71:φECOR71-10-R2 | R | X | X | R | X | S | X | S | X | R |
| 71-10 | 3 | ECOR71:φECOR71-10-R3 | R | S | S | S | S | S | S | S | S | S |
| 71-10 | 4 | ECOR71:φECOR71-10-R4 | R | S | S | R | S | S | S | S | S | S |
| 71-10 | 5 | ECOR71:φECOR71-10-R5 | R | S | R | R | R | S | S | R | S | S |
| 71-10 | 6 | ECOR71:φECOR71-10-R6 | R | S | R | R | R | S | S | S | S | S |
| 71-10 | 7 | ECOR71:φECOR71-10-R7 | R | S | S | S | S | S | S | X | S | S |
| 71-10 | 8 | ECOR71:φECOR71-10-R8 | R | S | S | S | S | S | S | S | S | S |
| 14-1 | 1 | ECOR71:φECOR14-1-R1 | R | S | S | R | R | R | S | S | R | X |
| 14-1 | 2 | ECOR71:φECOR14-1-R2 | R | S | R | R | R | S | S | S | S | S |
| 14-1 | 3 | ECOR71:φECOR14-1-R3 | S | S | S | S | S | S | S | S | S | S |
| 14-1 | 4 | ECOR71:φECOR14-1-R4 | R | S | S | S | S | S | S | S | S | S |
| 14-1 | 5 | ECOR71:φECOR14-1-R5 | R | S | S | S | S | S | S | S | S | S |
| 14-1 | 6 | ECOR71:φECOR14-1-R6 | R | S | S | S | S | S | S | S | S | S |
| 14-1 | 7 | ECOR71:φECOR14-1-R7 | R | S | S | R | R | S | S | S | S | S |
| 14-1 | 8 | ECOR71:φECOR14-1-R8 | R | S | S | S | S | S | S | S | S | S |
| KIF | 1 | EV36:φK1F-R1 | S | S | S | S | S | S | S | S | S | S |
| KIF | 2 | EV36:φK1F-R2 | S | S | S | S | S | S | S | S | S | S |
| KIF | 3 | EV36:φK1F-R3 | S | S | S | R | S | S | S | S | S | S |
| KIF | 4 | EV36:φK1F-R4 | S | S | S | S | S | S | S | S | S | S |
| KIF | 5 | EV36:φK1F-R5 | R | R | R | S | R | S | R | S | S | S |
| KIF | 6 | EV36:φK1F-R6 | S | T | R | R | R | R | S | S | S | S |
| KIF | 7 | EV36:φK1F-R7 | S | S | S | S | S | S | S | S | S | S |
| KIF | 8 | EV36:φK1F-R8 | S | S | S | S | S | S | S | S | S | X |

R: Resistant; S: Sensitive; T: Transient (defined initially as being sensitive followed by emergences of a resistant phenotype); X: Not determined; Bold and underlined boxes indicate results from clones which were re-challenged with the indicated bacteriophage used to isolate the original putatively resistant clone.

In other cases, individual clones were readily identified with stable phenotypic resistance to the original challenge phage. However, each of these clones, while resistant to the original wild-type phage, retained sensitivity to at least one of the phages in the panel. Only one was tested per initial phage challenge. In some instances, resistance profiles across phages and for individual phages change dependent on the tested.

### Example 5: Identification of Lead Bacteriophages for a Cocktail

Potential wild-type bacteriophages and combinations therein were evaluated using data from individual wild-type phage host range and resistance results in Table 2 and Table 3, respectively, against the following parameters: 1) phages have collective activity against approximately 90% of the clinical isolate panel or greater; 2) phages result in infection of each by at least 2 phages within the cocktail, intended to ensure sensitivity to the cocktail in the event of resistance to any single bacteriophage; 3) phages do not generate primary resistance in more than 50% of clones isolated, with primary resistance defined as emergence of escape clones after challenge with an individual phage that retain resistance to that particular phage after clonal outgrowth and re-challenge, and lastly 4) phages do not generate cross-resistance to all other phages in the cocktail, defined as emergence of escape clones after initial challenge and clonal outgrowth that display sustained resistance to the initial challenge phage and novel resistance to other phages in the proposed cocktail to which that clone was previously treatment-naïve.

Wild-type bacteriophage combinations shown in Table 2 (cumulative host range of approximately 94%) were compared to resistance profiles shown in Table 3. From these data, and based on the optimization parameters described above, five candidate bacteriophages have been identified with an approximately 94% host range coverage when tested against a panel of 18 genetically diverse E. coli isolates, with each having susceptibility to a minimum of 2 of the 5 phages. Each wild-type phage is predicted to result in less than 50% emergent clones displaying primary resistance and no clones that display resistance to all phages in the proposed cocktail. These data are summarized in Table 4, Table 5, and Table 6 and form the basis for preliminary development of a crPhage cocktail. A single crRNA expression construct is designed such that it directs the activity Type I-E and Type I-F CRISPR-Cas3 systems present in approximately 78% of E. coli genomes assessed (n=625) to target the host E. coli chromosome at multiple highly conserved loci collectively present in >99% of assessed genomes. Each individual crRNA was validated in vitro by transformation into E. coli isolates to demonstrate activity of each individual crRNA against targeted E. coli sequences. These crRNAs are then assembled into arrays that are expressed from a bacteriophage genome and processed by endogenous CRISPR-Cas systems to target the host chromosome.

The proposed wild-type bacteriophages are currently being evaluated for the absence of lysogeny, virulence genes or antibiotic resistance genes as outlined in Table 1.

TABLE 4
Host range of 5 individual bacteriophages for proposed crPhage cocktail.

| | | | | | | | # sensitive |
|---|---|---|---|---|---|---|---|
| | | | | | | | phages in |
| | | ΦECOR71-3 | ΦECOR71-7 | ΦECOR71-10 | ΦECOR14-1 | ΦK1F*** | cocktail |
| E. coli | ECOR2 | 1 | 1 | 0 | 0 | 0 | 2/5 |
| Strain | ECOR5 | 1 | 1 | 0 | 0 | 0 | 2/5 |
| | ECOR14 (UTI Isolate) | 0 | 0 | 1 | 1 | 0 | 2/5 |
| | ECOR21 | 1 | 1 | 0 | 0 | 0 | 2/5 |
| | ECOR27 | 1 | 1 | 0 | 0 | 0 | 2/5 |
| | ECOR29 | 1 | 1 | 0 | 0 | 0 | 2/5 |
| | ECOR35 | 0 | 0 | 0 | 1 | 1 | 2/5 |
| | ECOR36 | 0 | 0 | 0 | 1 | 1 | 2/5 |
| | ECOR41 | 0 | 0 | 1 | 1 | 1 | 3/5 |
| | ECOR47 | 1 | 1 | 1 | 1 | 0 | 4/5 |
| | ECOR51 | 0 | 1 | 1 | 0 | 0 | 2/5 |
| | ECOR56 | 1 | 1 | 1 | 1 | 0 | 4/5 |
| | ECOR58 | 1 | 1 | 0 | 0 | 0 | 2/5 |
| | ECOR62 (UTI Isolate) | 1 | 1 | 1 | 0 | 1 | 4/5 |
| | ECOR64 (UTI Isolate) | 1 | 1 | 0 | 0 | 0 | 2/5 |
| | ECOR71 (UTI Isolate) | 1 | 1 | 1 | 1 | 1 | 5/5 |
| | EV36 | 1 | 1 | 1 | 1 | 1 | 5/5 |
| | CFT073 (UPEC Strain) | 0 | 0 | 0 | 0 | 0 | 0/5 |
| | # strains sensitive to phage | 12/18 | 13/18 | 8/18 | 8/18 | 6/18 | 17/18 |
| | % strains sensitive to phage | 67% | 72% | 44% | 44% | 33% | 94.4% |

Bold text rows demonstrated preliminary host range coverage against uropathogenic E. coli isolates.

TABLE 5
Resistance profiles for 5 individual bacteriophages for a proposed crPhage cocktail.
(Revised from Table 2 based on ECOR71 sensitivity to ΦK1F observed in Table 3)

| Initial Challenge Phage/Apparent | | | | | | | |
|---|---|---|---|---|---|---|---|
| Resistant Clone | | | | | | | |
| | | Resistant | Sensitivity after re-challenge with indicated phage | | | | |
| Phage | | Clone | ΦECOR71-3 | ΦECOR71-7 | ΦECOR71-10 | ΦECOR14-1 | ΦK1F |
| ΦECOR71-3 | ECOR71 | 1 | Y | Y | Y | Y | Y |
| ΦECOR71-3 | ECOR71 | 2 | Y | Y | Y | Y | Y |
| ΦECOR71-3 | ECOR71 | 3 | Y | Y | Y | Y | Y |
| ΦECOR71-3 | ECOR71 | 4 | Y | Y | N | N | N |
| ΦECOR71-3 | ECOR71 | 5 | Y | Y | Y | Y | Y |
| ΦECOR71-3 | ECOR71 | 6 | Y | Y | Y | Y | Y |
| ΦECOR71-3 | ECOR71 | 7 | Y | Y | N | Y | Y |
| ΦECOR71-3 | ECOR71 | 8 | Y | Y | Y | Y | Y |
| ΦECOR71-7 | ECOR71 | 1 | Y | Y | Y | Y | Y |
| ΦECOR71-7 | ECOR71 | 2 | Y | Y | Y | Y | Y |
| ΦECOR71-7 | ECOR71 | 3 | Y | Y | Y | Y | Y |
| ΦECOR71-7 | ECOR71 | 4 | Y | Y | N | Y | Y |
| ΦECOR71-7 | ECOR71 | 5 | Y | Y | Y | Y | Y |
| ΦECOR71-7 | ECOR71 | 6 | Y | Y | Y | Y | Y |
| ΦECOR71-7 | ECOR71 | 7 | N | Y | N | N | Y |
| ΦECOR71-10 | ECOR71 | 1 | Y | Y | Y | Y | Y |
| ΦECOR71-10 | ECOR71 | 2 | Y | Y | Y | Y | Y |
| ΦECOR71-10 | ECOR71 | 3 | Y | Y | N | Y | Y |
| ΦECOR71-10 | ECOR71 | 4 | Y | Y | Y | Y | Y |
| ΦECOR71-10 | ECOR71 | 5 | Y | Y | Y | Y | Y |
| ΦECOR14-1 | ECOR71 | 1 | Y | Y | Y | Y | Y |
| ΦECOR14-1 | ECOR71 | 2 | Y | Y | Y | Y | Y |
| ΦECOR14-1 | ECOR71 | 3 | Y | Y | Y | Y | Y |
| ΦECOR14-1 | ECOR71 | 4 | Y | Y | Y | Y | Y |
| ΦECOR14-1 | ECOR71 | 5 | Y | Y | Y | Y | Y |
| ΦECOR14-1 | ECOR71 | 6 | Y | Y | Y | Y | Y |
| ΦECOR14-1 | ECOR71 | 7 | Y | Y | Y | Y | Y |
| ΦK1F | EV36 | 1 | Y | Y | Y | Y | Y |
| ΦK1F | EV36 | 2 | Y | Y | Y | Y | Y |
| ΦK1F | EV36 | 3 | Y | Y | Y | Y | Y |
| ΦK1F | EV36 | 4 | Y | Y | Y | Y | Y |
| ΦK1F | EV36 | 5 | N | Y | Y | Y | Y |
| ΦK1F | EV36 | 6 | Y | N | Y | Y | Y |
| ΦK1F | EV36 | 7 | Y | Y | Y | Y | Y |

Bold and underlined boxes indicate results from clones which were re-challenged with the indicated bacteriophage used to isolate the original putatively resistant clone.

TABLE 6
Summary data for 5 individual bacteriophages for proposed crPhage cocktail.

| | | | | Resistance Data | | | |
|---|---|---|---|---|---|---|---|
| | | | | # Primary | | | Min. # |
| Component | | Host Range | | Resistant | # Total | % Primary | phages |
| name | Phage | # s | % s | Clones* | Clones* | Resistance* | per** |
| LBx-UT01-ECΦ1 | ΦECOR71-3 | 12/18 | 66.7% | 0 | 8 | 0.0% | 2 |
| LBx-UT01-ECΦ2 | ΦECOR71-7 | 13/18 | 72.2% | 0 | 7 | 0.0% | 2 |
| LBx-UT01-ECΦ3 | ΦECOR71-10 | 8/18 | 44.4% | 1 | 7 | 14.3% | 4 |
| LBx-UT01-ECΦ4 | ΦECOR14-1 | 8/18 | 44.4% | 1 | 8 | 12.5% | 5 |
| LBx-UT01-ECΦ5 | ΦK1F | 6/18 | 33.3% | 0 | 7 | 0.0% | 4 |
| LBx-UT01 | Cocktail | 17/18 | 94.4% | | | | |

*For resistance to primary challenge phage, excluded single data points where phage sensitivity data could not be determined (N = 3).
**For cocktail susceptibility, excluded data for 6 resistant isolate as some individual phage library data points could not be determined (N = 6).

The proposed crPhage cocktail is further summarized in Table 7 below:

TABLE 7
Summary of proposed preliminary crPhage cocktail.

| crPhage name | Phage type | Replication host | Isolation source | Proposed concentration |
|---|---|---|---|---|
| LBx-UT01-ECΦ1 | Obligate | E. coli strain | Durham, NC Wastewater | 10⁹-10¹¹ PFU/mL |
| | lytic | ECOR71 | Treatment Plant | |
| LBx-UT01-ECΦ2 | Obligate | E. coli strain | Durham Wastewater | 10⁹-10¹¹ PFU/mL |
| | lytic | ECOR71 | Treatment Plant | |
| LBx-UT01-ECΦ3 | Obligate | E. coli strain | Durham Wastewater | 10⁹-10¹¹ PFU/mL |
| | lytic | ECOR71 | Treatment Plant | |
| LBx-UT01-ECΦ4 | Obligate | E. coli strain | Durham Wastewater | 10⁹-10¹¹ PFU/mL |
| | lytic | ECOR71 | Treatment Plant | |
| LBx-UT01-ECΦ5 | Obligate | E. coli strain | External source | 10⁹-10¹¹ PFU/mL |
| | lytic | EV36 | (K1F phage) | |

The composition of the crRNA arrays for the proposed crPhage cocktail are summarized in Table 8 below:

TABLE 8
Summary of individual crRNA information.

| | | Compatible | Target | Target gene | | |
|---|---|---|---|---|---|---|
| Array | crRNA name | Cas system | gene name | function | Target sequence | PAM |
| LBx-UT01- | LBx-UT01-EC- | Type I-E | acpP | Lipid | SEQ ID NO. 3 | GAG |
| EC-IEa | IE_spacer1 | | | biosynthesis | ATTCCGGACGAAGAAGCTGAGAAAATCACCAC | |
| LBx-UT01- | LBx-UT01-EC- | Type I-E | gapA1 | Glycolysis | SEQ ID NO. 4 | AGG |
| EC-IEa | IE_spacer2 | | | | TATCAACGGTTTTGGCCGTATCGGTCGCATTG | |
| LBx-UT01- | LBx-UT01-EC- | Type I-E | secY1 | Secretory | SEQ ID NO. 5 | AAG |
| EC-IEa | IE_spacer3 | | | | TGCAAACTCTGATGATGTCCAGTCAGTATGAG | |
| LBx-UT01- | LBx-UT01-EC- | Type I-E | tsf | Translation | SEQ ID NO. 6 | GAG |
| EC-IEa | IE_spacer4 | | | | AAAATGGTTGAAGGCCGCATGAAGAAATTCAC | |
| LBx-UT01- | LBx-UT01-EC- | Type I-F | csrA | Glycolysis | SEQ ID NO. 7 | CC |
| EC-IFa | IF_spacer1 | | | | AGGCTGAAAAATCCCAGCAGTCCAGTTACTA | |
| LBx-UT01- | LBx-UT01-EC- | Type I-F | ftsA | Cell division | SEQ ID NO. 8 | CC |
| EC-IFa | IF_spacer2 | | | | GTATTATTCGACGGCGGTGGGATTGCTTCAC | |
| LBx-UT01- | LBx-UT01-EC- | Type I-F | fusA | Translation | SEQ ID NO. 9 | CC |
| EC-IFa | IF_spacer3 | | | | AAAGCTGACCAGGAAAAAATGGGTCTGGCTC | |
| LBx-UT01- | LBx-UT01-EC- | Type I-F | secY | Secretory | SEQ ID NO. 10 | CC |
| EC-IFa | IF_spacer4 | | | | GCTTTATGTGTTACTCTATGCGTCTGCAATC | |

### Example 6: Optimizing the CRISPR Array for Lethality

To develop effective CRISPR-enhanced bacteriophages for use against C. difficile, various modifications were made to the CRISPR array.

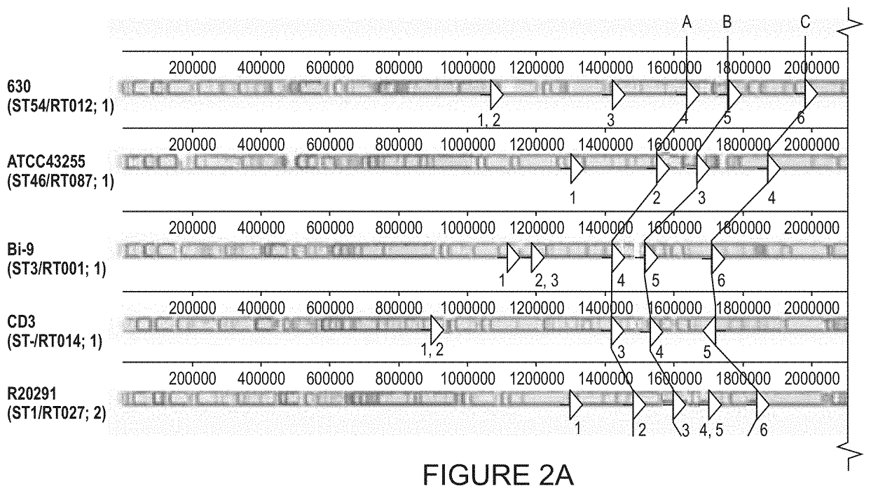

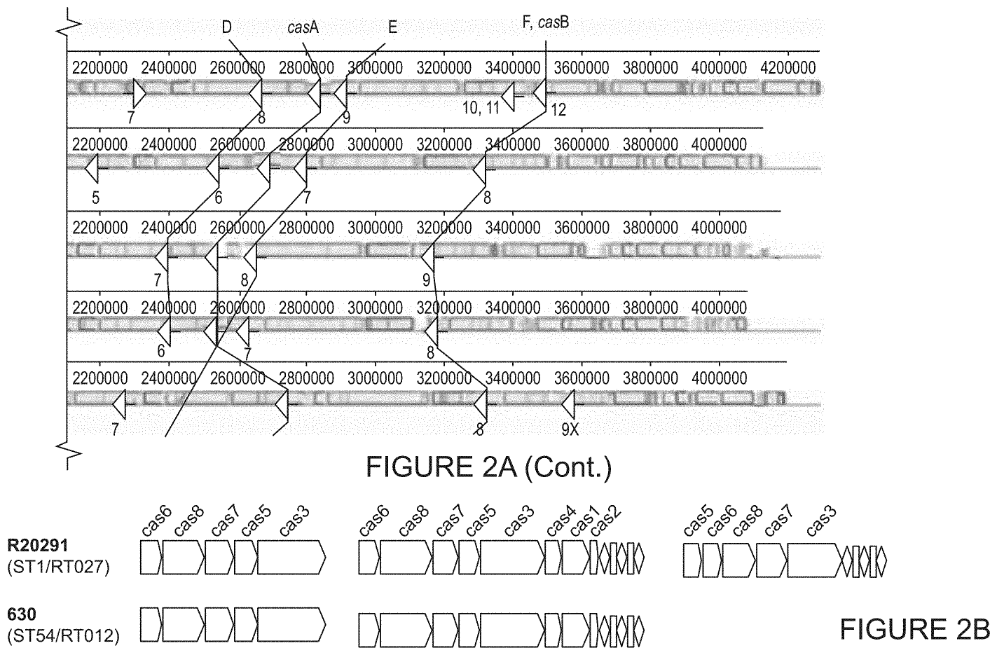

FIG. 2A shows a schematic diagram of the linear alignment of the identified CRISPR-Cas systems from within five strains of C. difficile with identification of the various CRISPR-Cas constituent components. The CRISPR-Cas system operon structures for strains 630 and R20291 are further diagrammed in FIG. 2B.

Initial crRNA arrays within the C. difficile targeting crPhages comprised a native leader sequence for a CRISPR array from C. difficile 630 or R20291 that were combined with the consensus repeat sequences from the native CRISPR arrays found in C. difficile. The spacer sequence was defined by the consensus PAM sequence for the endogenous Type I-B systems in C. difficile and complementary to three selected C. difficile target host genes including: dmsB, phi1, and phi2. Two additional configurations were tested including: a crRNA array using an endogenous enolase promotor in lieu of the native leader sequence and a crRNA array wherein the second repeat sequences was changed to facilitate IDT synthesis (R2 A>G). The configuration of the CRISPR-RNA for engineering the bacteriophage is thus leader-repeat-spacer-repeat. The engineered bacteriophage was created through homologous recombination in a native bacterial host as a lysogen using democratized plasmids for genetic manipulation of Clostridia.

Clostridium difficile strains 630 and R20291 were grown over night in brain heart infusion (BHI) medium at 3T C in an anaerobic environment and then sub-cultured into 5 mL of fresh BHI at a 1% (vol/vol) inoculum. C. difficile strains were then incubated until an OD of 0.2 before beginning a CFU reduction assay. All preparation and handling of bacteriophages was performed as described previously. To each culture, a total MOI of 10 wild-type or CRISPR phage lysate was added with a final concentration of 10 mM MgCl₂ and 1 mM CaCl₂. OD and CFU were then monitored over the course of 6 hours.

Similarly, alterations to the nucleotide composition of the repeat sequence within the CRISPR array had marginal impact on the overall lethality of the crRNA. C. difficile strain 630 consensus SEQ ID NO: 11 of 5′-GTTTTATATTAACTATATGGAATGTAAAT-3′ was varied with single point nucleotide mutations for crRNAs comprising a native leader sequence for a CRISPR array from C. difficile 630 or R20291 or an endogenous enolase gene promoter and spacers complementary for dmsB or int C. difficile host genes. Changes in the repeat sequence are anticipated to alter the hairpin secondary structure which affects the overall crRNA activity. Table 9 below summarizes the changes in the crRNA lethality for the various constructs.

TABLE 9
Alterations in the crRNA array
repeat affect crRNA lethality.

| | | | Repeat two | |
|---|---|---|---|---|
| Spacer | Promoter | Length | (Alterations underlined) | Alog |
| dmsB | Leader | 36 | SEQ ID NO. 12 | 0.15 |
| | | | GTTTTAGATTAACTATATGGAATG | |
| | | | TAAAT | |
| dmsB | Enolase | 36 | SEQ ID NO. 12 | 0.01 |
| | | | GTTTTAGATTAACTATATGGAATG | |
| | | | TAAAT | |
| dmsB | Leader | 36 | SEQ ID NO. 13 | 0.00 |
| | | | GTTTTAGATTAACTATATGGAATG | |
| | | | TAAGT | |
| int | Leader | 36 | SEQ ID NO. 14 | 0.44 |
| | | | GTTTTAGATTAACTATATGGAATG | |
| | | | TAAGT | |
| int | Leader | 36 | SEQ ID NO. 15 | |
| | | | GTTTTAGATTAACTATGTGGAATG | |
| | | | TAAAT | |
| int | Leader | 34 | SEQ ID NO. 16 | |
| | | | GTTTTAGATTAACTATATGGAATG | |
| | | | TAAGT | |
| int | Leader | 35 | SEQ ID NO. 17 | |
| | | | GTTTTAGATTAACTATATGGAATG | |
| | | | TAAGT | |
| int | Leader | 37 | SEQ ID NO. 18 | |
| | | | GTTTTAGATTAACTATATGGAATG | |
| | | | TAAGT | |
| int | Leader | 38 | SEQ ID NO. 19 | |
| | | | GTTTTAGATTAACTATATGGAATG | |
| | | | TAAGT | |

### Example 7: crPhages have Enhanced Killing Activity

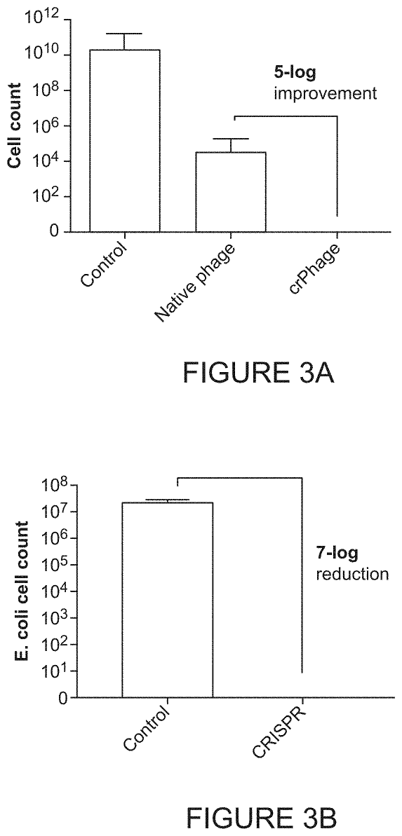

CRISPR-enhanced bacteriophages against E. coli and C. difficile were developed from distinct obligate lytic bacteriophages that contain an identical DNA sequence encoding a functional self-targeting CRISPR RNA embedded in the wild-type phage genome. As seen in FIG. 3A, treatment of an E. coli culture containing 10¹⁰ bacterial cells with the native, unmodified phage resulted in a 5-log reduction of bacterial cells by lytic activity of the phage. Treatment with the modified crPhage results in a further approximate 5-log improvement in killing activity of the E. coli. In some case, the improvement in anti-microbial activity is independent of the innate lytic activity of the phage and is a result of the anti-microbial activity of the CRISPR array itself. Treatment with the CRISPR array shows an approximate 7-log reduction in the bacterial cell population as seen in FIG. 3B.

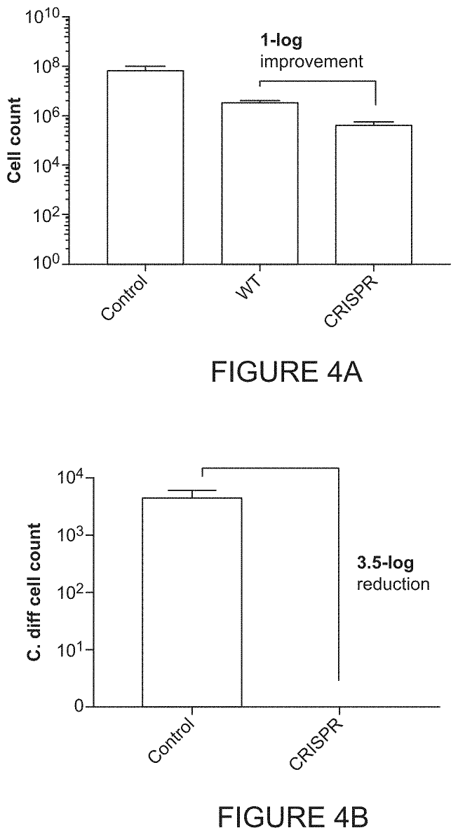

Similarly, treatment of a C. difficile culture containing approximately 10⁸ bacterial cells with the native, unmodified phage resulted in almost an approximate 1.5-log reduction of bacterial cells by lytic activity of the phage. An additional 1-log reduction in killing activity of the bacterial cells was seen when treated with the modified crPhage as seen in FIG. 4A. In some cases, the improvement in anti-microbial activity is independent of the innate lytic activity of the phage but is instead a result of the anti-microbial activity of the CRISPR array itself. Treatment with the CRISPR array only showed an approximate 3.5-log reduction in the C. difficile cell population as seen in FIG. 4B.

### Example 8: crPhages have Expanded Host Range and Associated Killing Activity

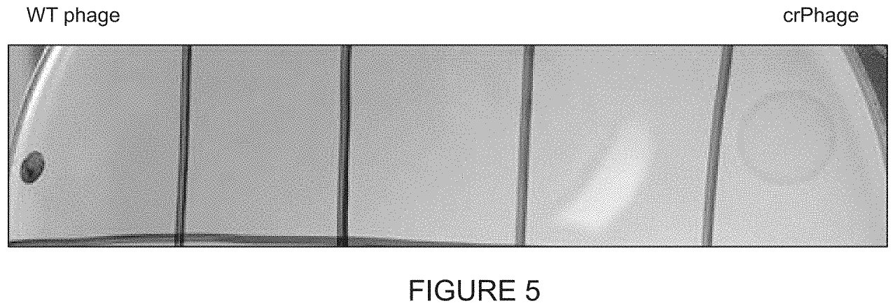

Identical concentrations of wild-type or crPhage were spotted onto an agar plate seeded with C. difficile strain 069 to identify crPhage sensitivity. Presence of phage plaques on the C. difficile bacterial lawn plate was used to identify C. difficile strains that were sensitive to killing by crPhage ϕCD146 but were insensitive to the wild-type phage ϕCD146 as seen in FIG. 5. Cell death by the crPhage ϕCD146 but not the wild-type phage ϕCD146 is indicative of bacterial cell death being independent of phage based lytic activity. Instead, it is representative of bacterial cell death by the CRISPR array to C. difficile strains that are natively insensitive to lytic phage ϕCD146 infection. The crRNA array used targeted R20291-3.

### Example 9: crPhages have Enhanced Killing Activity Over a Wide Range of C. difficile Strains

The ability of a crPhage to be effective over a panel of different strains for C. difficile was determined. The CRISPR-RNA was designed by incorporating a native leader sequence for a CRISPR array from Clostridium difficile R20291 and then combined with the consensus repeat sequences from the native CRISPR arrays found in C. difficile. The spacer sequence was defined by the consensus PAM sequence for the endogenous Type I-B systems in C. difficile and the length was determined by the most represented spacer length found in endogenous CRISPR arrays in C. difficile. The configuration of the CRISPR-RNA for engineering the bacteriophage is thus leader-repeat-spacer-repeat. The engineered bacteriophage was created through homologous recombination in a native bacterial host as a lysogen using democratized plasmids for genetic manipulation of Clostridia.

Clostridium difficile strains were grown over night in brain heart infusion (BHI) medium at 3T C in an anaerobic environment and then sub-cultured into 5 mL of fresh BHI at a 1% (vol/vol) inoculum. C. difficile strains were then incubated until an OD of 0.1 for the OD reduction assay and OD of 0.2 for the CFU reduction assay. All preparation and handling of bacteriophages was performed as described previously. To each culture, a total MOI of 10 wild-type or CRISPR phage lysate was added with a final concentration of 10 mM MgCl₂ and 1 mM CaCl₂. OD and CFU were then monitored over the course of 6 hours.

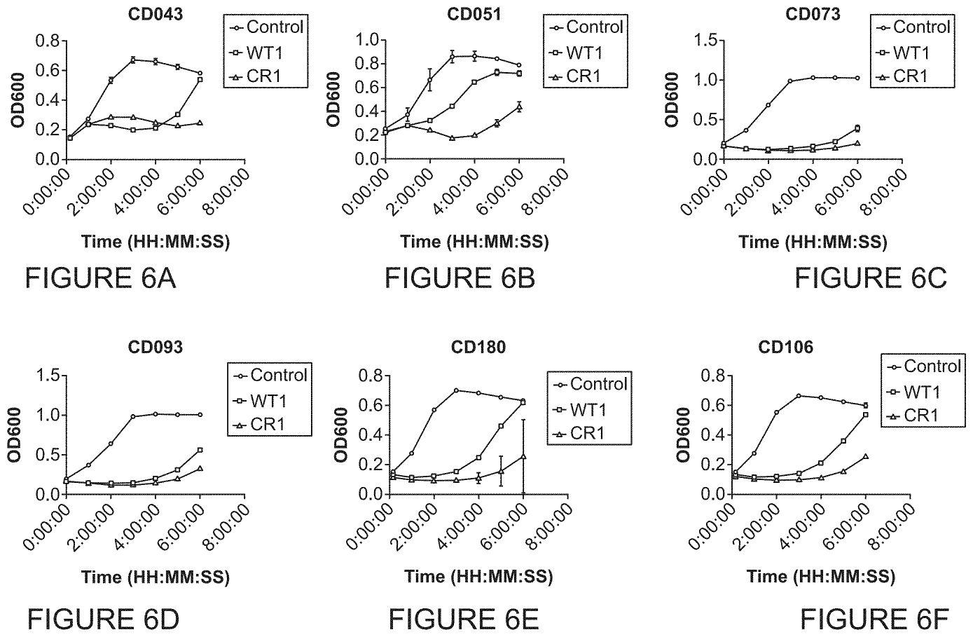

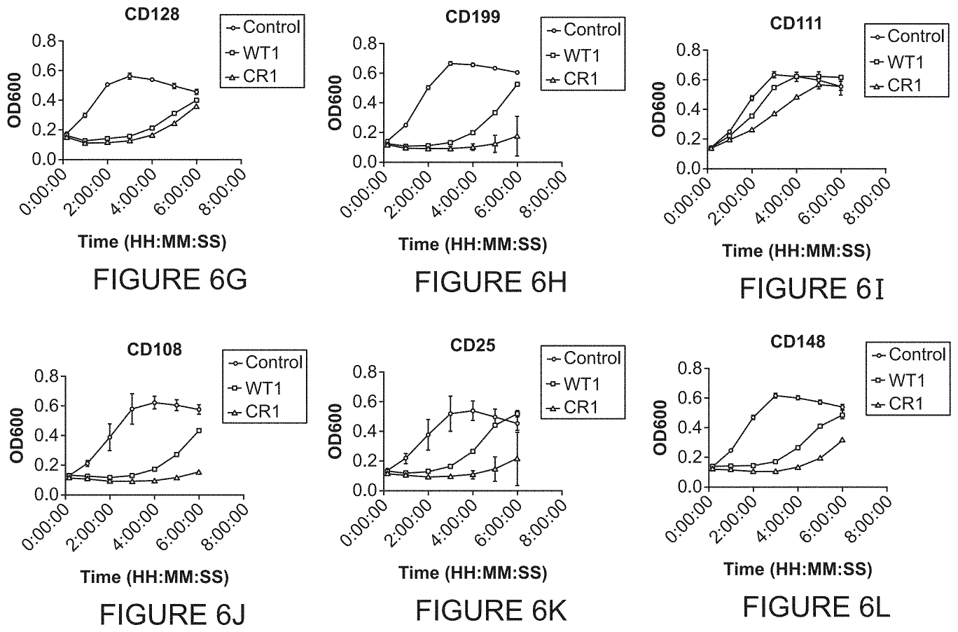

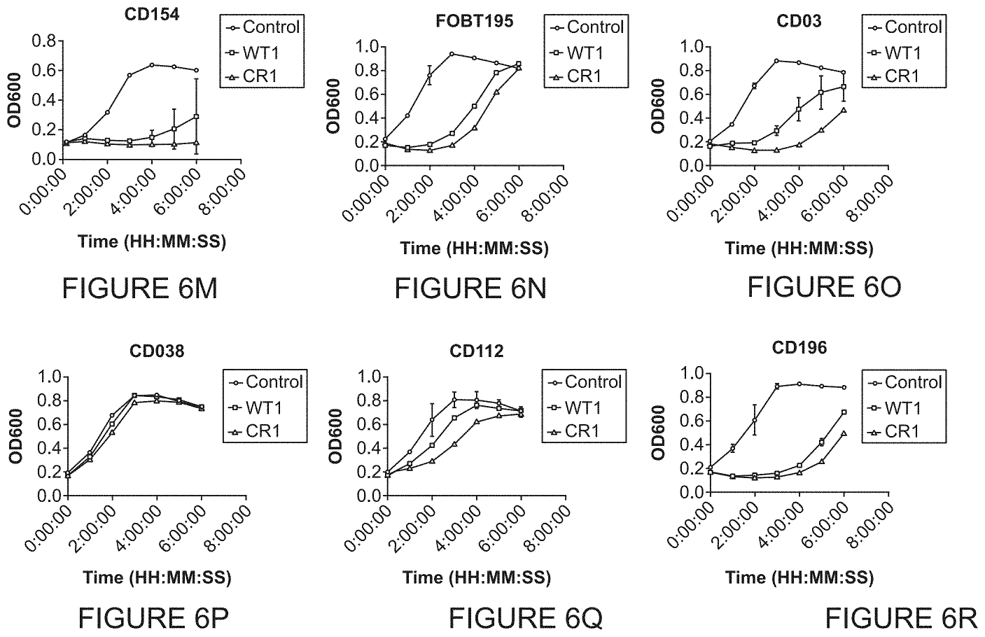

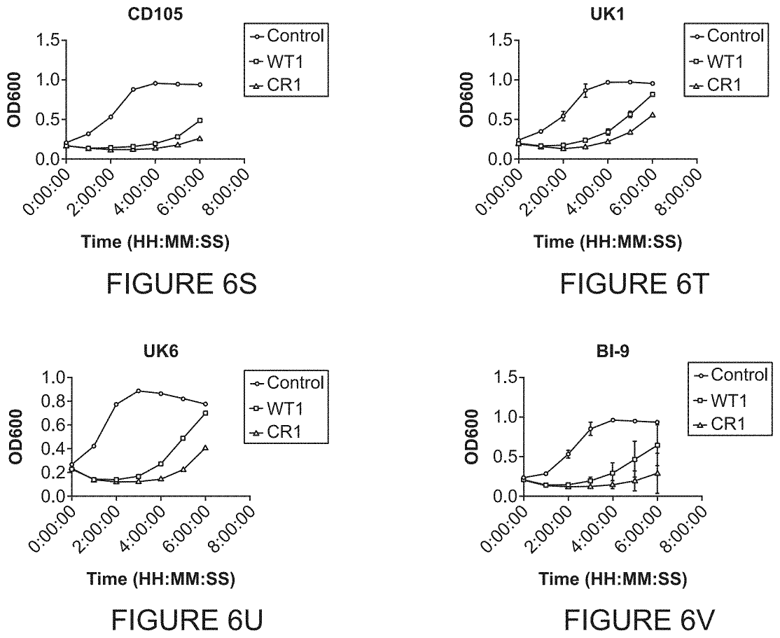

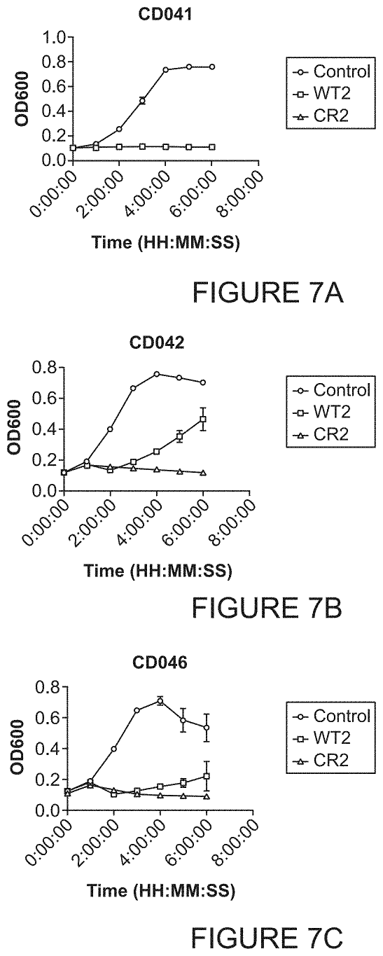

Comparison of wild-type phages against two crPhage variants was measure by optical density (OD600 nm) against C. difficile strains 1-22 for crPhage ϕCD146 as shown in FIG. 6A-FIG. 6V. crPhage ϕCD24-2, an additional variant, was likewise tested against C. difficile strains 23-25 as shown in FIG. 7A-FIG. 7C.

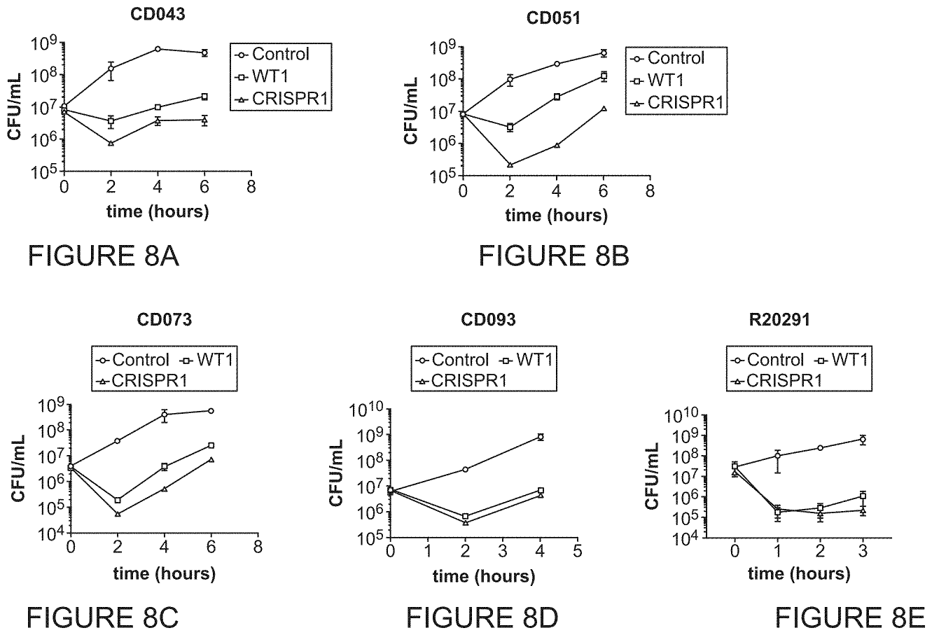

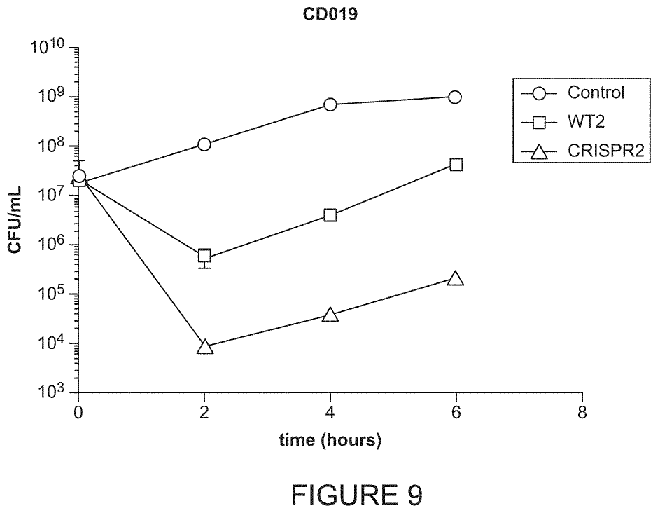

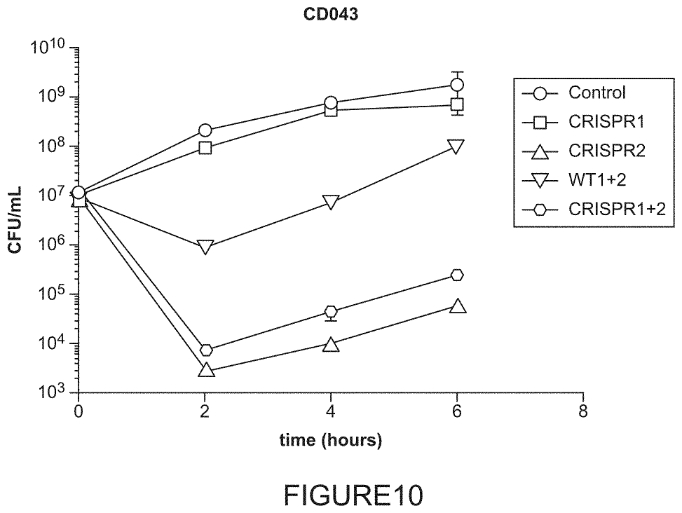

A subset of selected strains were further examined using colony forming unit (CFU) enumeration to compare the bacterial killing activity of crPhage ϕCD146 and 2. CFU assays for C. difficile strains 1˜4 against wild-type and crPhage ϕCD146 are shown in FIG. 8A-FIG. 8D. FIG. 9 shows a CFU assay for strain CD19 against wild-type and crPhage ϕCD24-2. A combinatorial comparison of crPhage ϕCD146 and crPhage ϕCD24-2 was tested in FIG. 10. A CFU assay of crPhage ϕCD146 and crPhage ϕCD24-2 anti-bacterial activity was conducted for each crPhage individually as well as the when administered together. Co-administration showed improved killing efficacy as compared to treatment with a combination of both wild-type phages together. Although wild-type phage ϕCD24-2 demonstrated the stronger anti-bacterial killing activity as compared to wild-type phage ϕCD146, when administered together, the killing combined efficacy significantly diminished. This is suggestive of the wild-type ϕCD146 phage potentially interfering with the activity of wild-type phage ϕCD24-2. However, the combination of crPhage ϕCD146 and crPhage ϕCD24-2 together show equal to slightly improved bacterial killing ability as compared to that of wild-type phage ϕCD24-2 by itself.

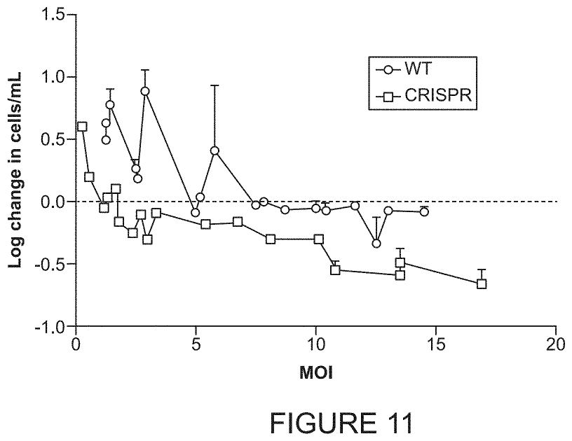

To evaluate the effects of the MOI against bacterial growth, cultures of C. difficile strain R20291 were grown and inoculated with wild-type ϕCD146 or crPhage ϕCD146 at various MOIs from 0 to 16. The combined effects of lytic activity and CRISPR array activity against R20291-3 upon the targeted host bacterium were evaluated. For both wildtype and crPhage, higher MOIs led to larger log reduction in the C. difficile. However, the crPhage ϕCD146 showed a consistent improvement in reducing the bacterial population compared to the wild-type phage ϕCD146 at all tested MOIs as showing in FIG. 11.

### Example 10: CRISPR Enhanced crPhages in Silico Design of CRISPR Array for Overcoming Resistance Rates

In order to determine potential resistance rates to CRISPR-based targeting, a mathematical model was developed to determine the frequency of mutation at any given potential target site of approximately 32 base pairs in a targeted genome:

$$\# of cells with mutations conferring survival = {( 1 - {( 1 - ( \frac{\frac{mutation rate}{genome}}{generation} ⋆ \frac{genome}{\# basepairs} ⋆ \# generations ) )}^{32} )}^{n} ⋆ ( CFU load )$$

Calculations estimating escape mutants in populations assumed the general rate of mutation for any given gene is 1 in 1000 mutations per genome per generation, that a typical bacterial genome is approximately 5×10⁶ base pairs in length, and that the total number of generations is estimated as the total length of the infection divided by the doubling time of organism. Thus, this equation estimates the total number of surviving cells as those that acquire mutations over the course of the infection in all potential spacer targets assuming an independent mutational rate for a given genome multiplied by the total number of genomes (e.g. cells) in the population.

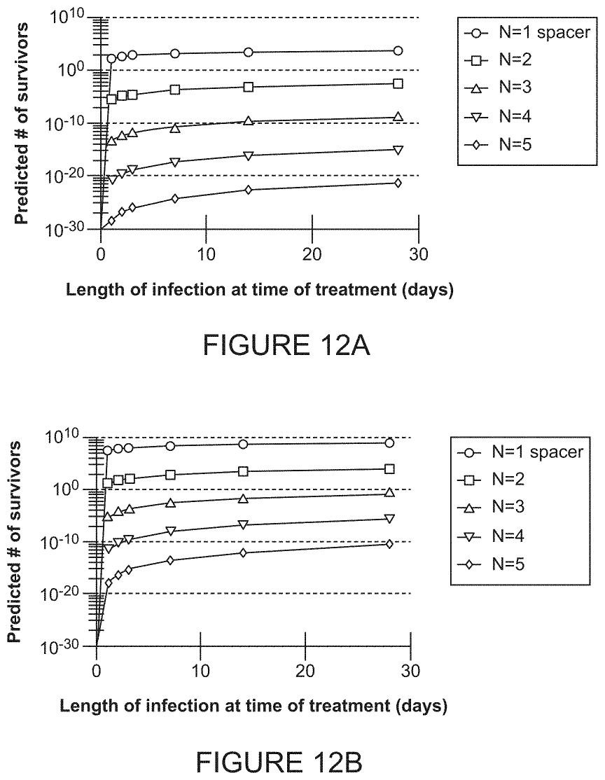

In silico prediction of number of resistant clones that emerge over time due to target site mutation as a function of the number of independent genes targeted by crRNAs. These models assume highly conservative assumptions that (1) the mutational rate is independent of gene target and (2) that all 32 bases of crRNA match for activity. Two sets of assumptions were tested to understand the impact of number of independently targeted genes on potential resistance to CRISPR. Two types of infection were modeled: acute infection rising to a total burden of 10¹⁰ CFU by doubling every 6 hours as seen in FIG. 12A or an aggressive infection to a total burden of 1014 CFU by doubling every 20 minutes as seen in FIG. 12B. Under these assumptions, both models show that 3 independent gene targets are sufficient to prevent mutational escape up to 28 days of infection length.

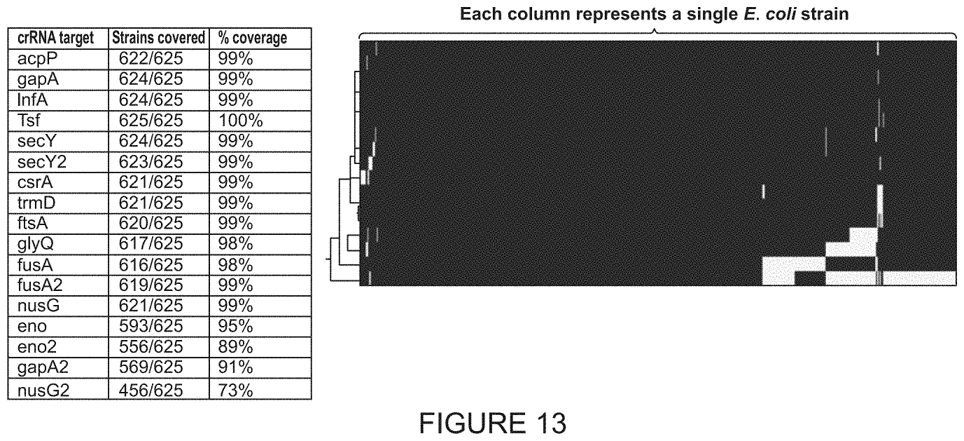

These estimates are considered to be conservative as: (1) crRNA targets are within highly conserved regions of essential genes and thus presumably be less likely to mutate than calculated here and (2) this model assumes that any single mutation in the 32 base pair crRNA target eliminates activity, contrary to data demonstrating that crRNAs tolerate 1 or more mismatches at its target site. CRISPR arrays were designed to express 4 independent crRNAs against 4 independent targets to ensure that resistance due to loss of CRISPR targets remains unlikely. To identify targets with the greatest prevalence amongst various E. coli strains, the strain coverage for each spacer target was analyzed as shown in FIG. 13. crRNA array targets include the following genes in order of highest to lowest percentage of strain coverage: Tsf (100%), cpP (99%), gapA (99%), infA (99%), secY (99%), secY′2 (99%), csrA (99%), trmD (99%), ftsA (99%), nusG (99%), fusA′2 (99%), fusA (98%), glyQ (98%), eno (95%), gapA′2 (91%), eno′2 (89%), and nusG′2 (73%).

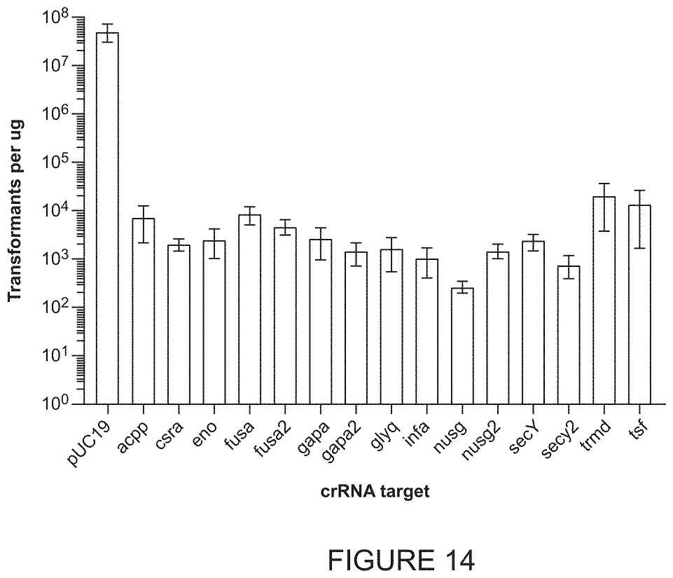

A series of individual Type I-E crRNAs targeted to conserved regions of the E. coli genome were constructed as seen in FIG. 14. crRNA targets include the following genes: acpP, csra, eno, fusA, gapA, glyQ, infA, nusG, secY, trmD, and Tsf. These individual Type I-E crRNAs constructs were tested by transformation of crRNA expression constructs into E. coli cells constitutively expressing a Type I-E CRISPR-Cas3 system with transformation efficiency calculated as transformants per microgram of input DNA and each data point is a single replicate from three independent experiments. Each tested crRNA resulted in similar observed levels of lethality when transformed into recipient E. coli. From these spacers, 4 Type I-E crRNAs were selected from each subset to assemble into final arrays.

### Example 11: crPhages Enhanced with LeuO Transcriptional Activator

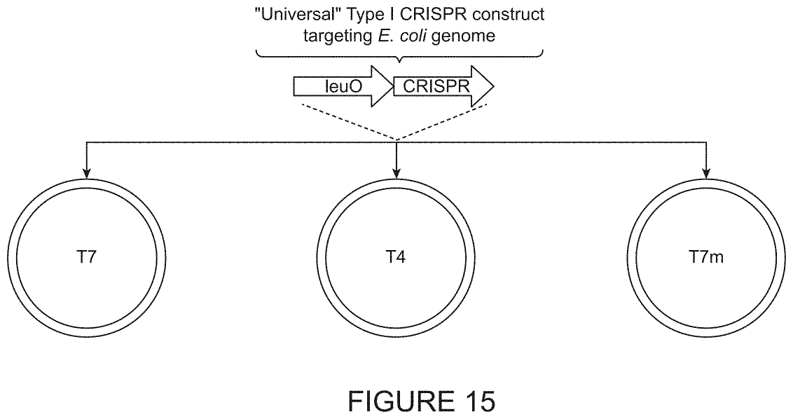

CRISPR-enhanced bacteriophages against E. coli were developed as a cocktail of up to three distinct obligate lytic bacteriophages that contain an identical DNA sequence encoding a functional self-targeting CRISPR RNA (crRNA cassette) embedded in the wild-type phage genome. Bacteriophages were engineered by one of two methods: (1) homologous recombination in E. coli cells with active phage infection or (2) by transformation of engineered phage DNA assembled outside of the cell to reconstitute active phages with engineered genomes. Each phage was engineered with a similar crRNA cassette that contained two elements: (1) a LeuO transcription factor gene derived from E. coli in front of a synthetic promoter, and (2) a repeat-spacer-repeat encoding a crRNA targeting the ftsA gene in front of a synthetic promoter. Three bacteriophage constructs were engineered: crT4, crT7, and crT7m. All three bacteriophage constructs are schematically illustrated in FIG. 15. The crRNA expression cassette used in the engineered various crPhages contains a single crRNA as shown in Table 10 below:

TABLE 10
crRNA expression cassette.

| | | | Target | Target | | |
|---|---|---|---|---|---|---|
| | crRNA | Compatible | gene | gene | | |
| Array | name | Cas system | name | function | Target sequence | PAM |
| ftsA | ftsA | Type I-E | ftsA | Cell | SEQ ID NO. 20 | AAG |
| | | | | division | AGGGTCTCACCAACTCGACGAGTCAGAATCAG | |

Bacteriophage crT4 was engineered by deleting the hoc gene and replacing with a crRNA cassette. Bacteriophage crT7 was engineered by deleting gp0.7, gp4.3, gp4.5 and gp4.7 and replacing with a crRNA cassette. Bacteriophage crT7m was engineered by deleting gp0.6, gp0.65, gp0.7, gp4.3, and gp4.5 and replacing with a crRNA cassette. Based on the observation that all phages were successfully engineered after these deletions, it was concluded that these early phage genes were non-essential for phage survival. Details of these engineered bacteriophages are summarized in Table 11 below:

TABLE 11
Summary of engineered crPhages.

| crPhage name | Phage type | Replication host | Isolation source |
|---|---|---|---|
| crT7m | Obligate | E. coli B-strain | Commercial |
| | lytic | | (ATCC T7m) |
| crT4 | Obligate | E. coli B-strain | Commercial |
| | lytic | | (ATCC T4) |
| crT7 | Obligate | E. coli B-strain | Commercial |
| | lytic | | (ATCC T7) |

Upon DNA transduction during infection, LeuO is expressed from the phage genome and subsequently upregulate expression of the endogenous Type I-E CRISPR-Cas3 operon in E. coli. Concurrently, the synthetic ftsA-targeting crRNA is expressed from the phage genome that is recognized and processed by the endogenous Type I-E CRISPR-Cas3 protein complex. This crRNA is then loaded onto a CRISPR-Cas3 complex and thereby directs the targeting and degradation of target bacterial DNA.

### Example 12: Prevalence and Distribution of CRISPR-Cas Systems in E. coli

There are a diverse range of CRISPR-Cas systems types and subtypes, with the majority (>60%) of discovered systems belonging to the Type I group that shares the unique feature of having the Cas3 signature nuclease. CRISPR-Cas3 systems are unique in that they generate single-strand nicks, followed by processive exonucleolytic degradation of targeted DNA. E. coli CRISPR-Cas systems belong to two distinct subtypes, Type I-E and Type I-F, that use this signature Cas3 nuclease for degradation.

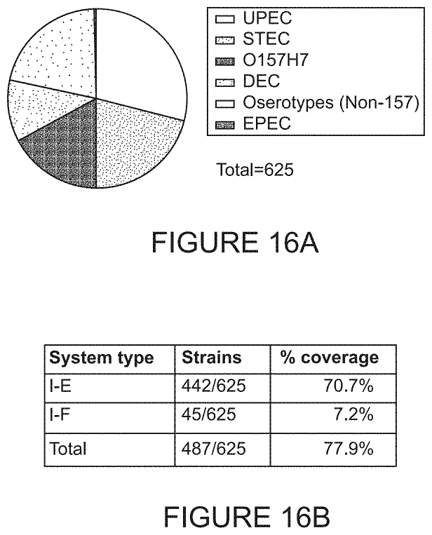

To determine an approximate distribution of CRISPR-Cas systems in E. coli, 625 publicly available E. coli genomes were analyzed, spanning a diversity of strains including: uropathogenic E. coli (UPEC), Shiga toxin producing E. coli, (STEC), O157:H7 serotype E. coli, diarrheagenic E. coli (DEC), non-157 O antigen type E. coli, and enteropathogenic E. coli (EPEC). Each genome was scanned for operons with similarity to canonical Type I-E or Type I-F E. coli CRISPR-Cas systems. FIG. 16A shows the relative amounts of each of these E. coli genomes. FIG. 16B shows that approximately 78% (487/625) of all strains already have the complete CRISPR-Cas3 system, either type I-E or type I-F. The proposed product exploits the presence of CRISPR-Cas3 proteins in the majority of E. coli by delivering only the guides and accessories required to activate endogenous CRISPR-Cas3 systems to target the genome.

### Example 13: Presence of LeuO Binding Sites Near Cas Operon in E. coli

To determine the prevalence of LeuO binding sites near the Cas operon in E. coli genome, 628 E. coli genomes were downloaded directly from NCBI with accession numbers, spanning a diversity of s including: uropathogenic E. coli (UPEC), Shiga toxin producing C. (STEC), various O-antigen: H-antigen serotype E. coli, diarrheagenic E. coli (DEC), and enteropathogenic E. coli (EPEC). Genomes were then queried for genes annotated as “CasB”. The CasB coding sequence and 5 kb flanking on either side were extracted for further annotation. Complete Cascade operons were determined by visual inspection for truncated genes in the CasABCDE or Cas3 genes. There were 401 intact Cascade-Cas3 operons detected (64% of all genomes queried). Next, 200-400 nucleotides upstream of Cas3 and downstream of Cas3 were extracted for analysis, respectively. Candidate LeuO binding sequences were retrieved and aligned to create a consensus sequence. Next, the individual and consensus sequences were queried against the Cas3 upstream and downstream sequences with a threshold nucleotide identity of 60%. Collectively, candidate LeuO binding sites were observed in 88.5% of strains containing an intact Cacade-Cas3 operon. Table 12 summarizes the search analysis of the E. coli genomes below:

TABLE 12
Summary of LeuO prevalence in E. coli genomes.

| E. coli Strain Designation | Accession No. | CRISPR | Cascade | Cas3-Cascade | LeuO site |
|---|---|---|---|---|---|
| DEC1A | NZ_AIEV00000000 | | | | |
| DEC1B | NZ_AIEW00000000 | | | | |
| DEC1C | NZ_AIEX00000000 | | | | |
| DEC1D | NZ_AIEY00000000 | | | | |
| DEC1E | NZ_AIEZ00000000 | | | | |
| DEC2B | NZ_AFJB00000000 | | | | |
| DEC2C | NZ_AIFB00000000 | | | | |
| DEC2D | NZ_AIFC00000000 | | | | |
| DEC2E | NZ_AIFD00000000 | | | | |
| DEC3A | NZ_AIFE00000000 | Yes | | Yes | Yes |
| DEC3B | NZ_AIFF00000000 | Yes | | Yes | Yes |
| DEC3C | NZ_AIFG00000000 | Yes | | Yes | Yes |
| DEC3D | NZ_AIFH00000000 | Yes | | Yes | Yes |
| DEC3E | NZ_AIFI00000000 | Yes | | Yes | Yes |
| DEC3F | NZ_AIFJ00000000 | Yes | | Yes | Yes |
| DEC4A | NZ_AIFK00000000 | Yes | | Yes | Yes |
| DEC4B | NZ_AIFL00000000 | Yes | | Yes | Yes |
| DEC4C | NZ_AIFM00000000 | Yes | | Yes | Yes |
| DEC4D | NZ_AIFN00000000 | Yes | | Yes | Yes |
| DEC4E | NZ_AIFO00000000 | Yes | | Yes | Yes |
| DEC4F | NZ_AIFP00000000 | Yes | | Yes | Yes |
| DEC5A | NZ_AIFQ00000000 | Yes | | Yes | Yes |
| DEC5B | NZ_AIFR00000000 | Yes | | Yes | Yes |
| DEC5C | NZ_AIFS00000000 | Yes | | Yes | Yes |
| DEC5D | NZ_AIFT00000000 | Yes | | Yes | Yes |
| DEC5E | NZ_AIFU00000000 | Yes | | Yes | Yes |
| DEC6A | NZ_AIFV00000000 | | | | |
| DEC6B | NZ_AIFW00000000 | | | | |
| DEC6E | NZ_AIFZ00000000 | Yes | | Yes | |
| DEC7A | NZ_AIGA00000000 | Yes | | Yes | Yes |
| DEC7B | NZ_AIGB00000000 | | | | |
| DEC7C | NZ_AIGC00000000 | Yes | | Yes | Yes |
| DEC7D | NZ_AIGD00000000 | Yes | | Yes | Yes |
| DEC7E | NZ_AIGE00000000 | Yes | | Yes | Yes |
| DEC8A | NZ_AIGF00000000 | Yes | | Yes | Yes |
| DEC8B | NZ_AIGG00000000 | Yes | | Yes | Yes |
| DEC8C | NZ_AIGH00000000 | Yes | | Yes | Yes |
| DEC8D | NZ_AIGI00000000 | Yes | | Yes | Yes |
| DEC8E | NZ_AIGJ00000000 | Yes | | Yes | Yes |
| DEC9A | NZ_AIGK00000000 | Yes | | Yes | Yes |
| DEC9B | NZ_AIGL00000000 | Yes | | Yes | Yes |
| DEC9C | NZ_AIGM00000000 | Yes | | Yes | Yes |
| DEC9D | NZ_AIGN00000000 | Yes | | Yes | Yes |
| DEC9E | NZ_AIGO00000000 | Yes | | Yes | Yes |
| DEC10E | NZ_AIGT00000000 | Yes | | Yes | Yes |
| DEC10F | NZ_AIGU00000000 | Yes | | Yes | Yes |
| DEC11A | NZ_AIGV00000000 | Yes | | Yes | Yes |
| DEC11B | NZ_AIGW00000000 | Yes | | Yes | Yes |
| DEC11C | NZ_AIGX00000000 | T | | | |
| DEC11D | NZ_AIGY00000000 | Yes | | Yes | Yes |
| DEC11E | NZ_AIGZ00000000 | Yes | | Yes | Yes |
| DEC12A | NZ_AIHA00000000 | Yes | | Yes | Yes |
| DEC12B | NZ_AIHB00000000 | Yes | | Yes | Yes |
| DEC12C | NZ_AIHC00000000 | Yes | | Yes | Yes |
| DEC12D | NZ_AIHD00000000 | Yes | | Yes | Yes |
| DEC12E | NZ_AIHE00000000 | Yes | | Yes | Yes |
| DEC13A | NZ_AIHF00000000 | Yes | | Yes | Yes |
| DEC13B | NZ_AIHG00000000 | Yes | | Yes | Yes |
| DEC13C | NZ_AIHH00000000 | Yes | | Yes | Yes |
| DEC13D | NZ_AIHI00000000 | Yes | | Yes | Yes |
| DEC13E | NZ_AIHJ00000000 | Yes | | Yes | Yes |
| DEC14A | NZ_AIHK00000000 | Yes | | Yes | Yes |
| DEC14B | NZ_AIHL00000000 | Yes | | Yes | |
| DEC14C | NZ_AIHM00000000 | Yes | | Yes | Yes |
| DEC14D | NZ_AIHN00000000 | Yes | | Yes | Yes |
| DEC15A | NZ_AIHO00000000 | Yes | | Yes | Yes |
| DEC15B | NZ_AIHP00000000 | Yes | | Yes | Yes |
| DEC15C | NZ_AIHQ00000000 | Yes | | Yes | Yes |
| DEC15D | NZ_AIHR00000000 | Yes | | Yes | Yes |
| DEC15E | NZ_AIHS00000000 | Yes | | Yes | Yes |
| EPECa12 | NZ_AKNH00000000 | Yes | | Yes | Yes |
| EPECa14 | NZ_ADUN00000000 | Yes | | Yes | Yes |
| EPEC C342-62 | NZ_AKNI00000000 | Yes | | Yes | Yes |
| O5:K4(L):H4 str. ATCC 23502 | NZ_CAPL00000000 | | | | |
| O6:H16:CFA/II str. B2C | NZ_AUZS00000000 | Yes | Yes | | Yes |
| O6:H16 str. 99-3165 | NZ_JHJW00000000 | Yes | Yes | | Yes |
| O6:H16 str. F5656C1 | NZ_JHJU00000000 | Yes | Yes | | Yes |
| O08 | NZ_AOGM00000000 | Yes | | Yes | Yes |
| O10:K5(L):H4 str. ATCC 23506 | NZ_CAPK00000000 | | | | |
| O15:H18 str. K1516 | NZ_JHJE00000000 | Yes | | Yes | Yes |
| O25:NM str. E2539C1 | NZ_JHJV00000000 | Yes | | Yes | |
| O26:H1 str. 2009C-4747 | NZ_JHGM00000000 | Yes | | Yes | Yes |
| O26:H11 str. 2009C-3612 | NZ_JHGZ00000000 | Yes | | Yes | Yes |
| O26:H11 str. 2011C-3655 | NZ_JHLN00000000 | Yes | | Yes | Yes |
| O28ac:NM str. 02-3404 | NZ_JHNY00000000 | | | | |
| O32:H37 str. P4 | NZ_AJQW00000000 | Yes | | Yes | |
| O39:NM str. F8704-2 | NZ_JHHJ00000000 | Yes | | Yes | Yes |
| O45:H2 str. 01-3147 | NZ_JHOA00000000 | | | | |
| O45:H2 str. 03-EN-705 | NZ_AGTK00000000 | | | | |
| O45:H2 str. 2009C-3686 | NZ_JHGY00000000 | | | | |
| O45:H2 str. 2009C-4780 | NZ_JHGJ00000000 | | | | |
| O45:H2 str. 2010C-3876 | NZ_JHFI00000000 | | | | |
| O45:H2 str. 2010C-4211 | NZ_JASS00000000 | | | | |
| O55:H7 str. 06-3555 | NZ_JHNL00000000 | Yes | | Yes | Yes |
| O55:H7 str. 3256-97 | NZ_AEUA00000000 | Yes | | Yes | Yes |
| O55:H7 str. USDA 5905 | NZ_AEUB00000000 | Yes | | Yes | Yes |
| O69:H11 str. 06-3325 | NZ_JHNP00000000 | Yes | | Yes | Yes |
| O69:H11 str. 07-3763 | NZ_JASN00000000 | Yes | | Yes | Yes |
| O69:H11 str. 07-4281 | NZ_JHLA00000000 | Yes | | Yes | Yes |
| O69:H11 str. 08-4661 | NZ_JHHG00000000 | Yes | | Yes | Yes |
| O69:H11 str. 2009C-3601 | NZ_JHHA00000000 | Yes | | Yes | Yes |
| O78:H12 str. 00-3279 | NZ_JFBE00000000 | | | | |
| O79:H7 str. 06-3501 | NZ_JHNM00000000 | Yes | | Yes | Yes |
| O81:NM str. 02-3012 | NZ_JHNZ00000000 | | | | |
| O91:H21 str. B2F1 | NZ_AGTI00000000 | Yes | | Yes | Yes |
| O104:H4 str. 11-02030 | NZ_AMVR00000000 | Yes | | Yes | Yes |
| O104:H4 str. 11-02033-1 | NZ_AMVS00000000 | Yes | | Yes | Yes |
| O104:H4 str. 11-02092 | NZ_AMVT00000000 | Yes | | Yes | Yes |
| O104:H4 str. 11-02093 | NZ_AMVU00000000 | Yes | | Yes | Yes |
| O104:H4 str. 11-02281 | NZ_AMVV00000000 | Yes | | Yes | Yes |
| O104:H4 str. 11-02913 | NZ_AMVX00000000 | Yes | | Yes | Yes |
| O104:H4 str. 11-03439 | NZ_AMVY00000000 | Yes | | Yes | Yes |
| O104:H4 str. 11-03943 | NZ_AMWA00000000 | Yes | | Yes | Yes |
| O104:H4 str. 11-04080 | NZ_AMVZ00000000 | Yes | | Yes | Yes |
| O104:H4 str. E112/10 | NZ_AHAV00000000 | Yes | | Yes | Yes |
| O104:H4 str. GOS1 | NZ_AFWO00000000 | Yes | | Yes | Yes |
| O104:H4 str. GOS2 | NZ_AFWP00000000 | Yes | | Yes | Yes |
| O104:H4 str. H112180280 | NZ_AFPN00000000 | Yes | | Yes | Yes |
| O104:H4 str. H112180282 | NZ_AFSO00000000 | | | | |
| O104:H4 str. ON2010 | NZ_AHZE00000000 | | | | |
| O104:H4 str. ON2011 | NZ_AHZF00000000 | | | | |
| O104:H4 str. TY-2482 | NZ_AFVR00000000 | Yes | | Yes | Yes |
| O104:H4 str. TY-2482 | NZ_AFOG00000000 | | | | |
| O104:H21 str. 94-3025 | NZ_JHJZ00000000 | | | | |
| O111:H11 str. CVM9455 | NZ_AKAX00000000 | Yes | | Yes | Yes |
| O111:H11 str. CVM9534 | NZ_AJVS00000000 | Yes | | Yes | Yes |
| O111:H11 str. CVM9545 | NZ_AJVT00000000 | Yes | | Yes | Yes |
| O111:H11 str. CVM9553 | NZ_AKAY00000000 | Yes | | Yes | Yes |
| O111:H8 str. CVM9570 | NZ_AJVU00000000 | Yes | | Yes | Yes |
| O111:H8 str. CVM9574 | NZ_AJVV00000000 | Yes | | Yes | Yes |
| O111:H8 str. CVM9602 | NZ_AKAV00000000 | Yes | | Yes | Yes |
| O111:H8 str. CVM9634 | NZ_AKAW00000000 | Yes | | Yes | Yes |
| O111:H11 str. CFSAN001630 | NZ_AMXP00000000 | Yes | | Yes | Yes |
| O111:NM str. 01-3076 | NZ_JFGU00000000 | Yes | | Yes | Yes |
| O111:NM str. 03-3484 | NZ_JHNU00000000 | Yes | | Yes | Yes |
| O111:NM str. 04-3211 | NZ_JHNS00000000 | Yes | | Yes | Yes |
| O111:NM str. 08-4487 | NZ_JHKU00000000 | Yes | | Yes | Yes |
| O111:NM str. 2009C-4006 | NZ_JHGU00000000 | Yes | | Yes | Yes |
| O111:NM str. 2009C-4052 | NZ_JHGS00000000 | Yes | | Yes | Yes |
| O111:NM str. 2010C-3053 | NZ_JHFZ00000000 | Yes | | Yes | Yes |
| O111:NM str. 2010C-3977 | NZ_JHFG00000000 | Yes | | Yes | Yes |
| O111:NM str. 2010C-4086 | NZ_JHFF00000000 | Yes | | Yes | Yes |
| O111:NM str. 2010C-4221 | NZ_JHFE00000000 | Yes | | Yes | Yes |
| O111:NM str. 2010C-4592 | NZ_JHMY00000000 | Yes | | Yes | Yes |
| O111:NM str. 2010C-4622 | NZ_JHMX00000000 | Yes | | Yes | Yes |
| O111:NM str. 2010C-4715 | NZ_JHMW00000000 | Yes | | Yes | Yes |
| O111:NM str. 2010C-4735 | NZ_JHMU00000000 | Yes | | Yes | Yes |
| O111:NM str. 2010C-4746 | NZ_JHMT00000000 | Yes | | Yes | Yes |
| O111:NM str. 2011C-3170 | NZ_JHMA00000000 | Yes | | Yes | Yes |
| O111:NM str. 2011C-3362 | NZ_JHLW00000000 | Yes | | Yes | Yes |
| O111:NM str. 2011C-3573 | NZ_JHLQ00000000 | Yes | | Yes | Yes |
| O111:NM str. 2011C-3632 | NZ_JHLO00000000 | Yes | | Yes | Yes |
| O111:NM str. 2011C-3679 | NZ_JHLM00000000 | Yes | | Yes | Yes |
| O111:NM str. K6904 | NZ_JHHN00000000 | Yes | | Yes | Yes |
| O111:NM str. K6908 | NZ_JHHM00000000 | Yes | | Yes | Yes |
| O111:NM str. K6915 | NZ_JHHL00000000 | Yes | | Yes | Yes |
| O113:H21 str. CL-3 | NZ_AGTH00000000 | Yes | | Yes | Yes |
| O118:H16 str. 07-4255 | NZ_JASP00000000 | Yes | | Yes | Yes |
| O119:H6 | NZ_BBUR00000000 | | | | |
| O119:H6 | NZ_BBUT00000000 | | | | |
| O119:H6 | NZ_BBUU00000000 | | | | |
| O119:H6 | NZ_BBUS00000000 | | | | |
| O121:H7 str. 2009C-3299 | NZ_JHHC00000000 | Yes | | Yes | Yes |
| O121:H19 str. 03-3227 | NZ_JHNX00000000 | | | | |
| O121:H19 str. 06-3003 | NZ_JHNR00000000 | | | | |
| O121:H19 str. 06-3822 | NZ_JHNH00000000 | | | | |
| O121:H19 str. 2009C-4050 | NZ_JHGT00000000 | | | | |
| O121:H19 str. 2009C-4659 | NZ_JHGN00000000 | | | | |
| O121:H19 str. 2009C-4750 | NZ_JHGL00000000 | | | | |
| O121:H19 str. 2009EL1302 | NZ_JHGH00000000 | | | | |
| O121:H19 str. 2009EL1412 | NZ_JHGG00000000 | | | | |
| O121:H19 str. 2010C-3609 | NZ_JHFM00000000 | | | | |
| O121:H19 str. 2010C-3794 | NZ_JHFL00000000 | | | | |
| O121:H19 str. 2010C-3840 | NZ_JHFK00000000 | | | | |
| O121:H19 str. 2010C-4254 | NZ_JHFC00000000 | | | | |
| O121:H19 str. 2010C-4824 | NZ_JHMO00000000 | | | | |
| O121:H19 str. 2010C-4966 | NZ_JHML00000000 | | | | |
| O121:H19 str. 2010C-4989 | NZ_JHMJ00000000 | | | | |
| O121:H19 str. 2010EL1058 | NZ_JHMG00000000 | | | | |
| O121:H19 str. 2011C-3537 | NZ_JHLR00000000 | | | | |
| O121:H19 str. 2011C-3609 | NZ_JASV00000000 | | | | |
| O121:H19 str. F6714 | NZ_JHJR00000000 | | | | |
| O121:H19 str. K5198 | NZ_JHIK00000000 | | | | |
| O121:H19 str. K5269 | NZ_JHIJ00000000 | | | | |
| O121:H19 str. MT#2 | NZ_AGTJ00000000 | | | | |
| O123:H11 str. 2009C-3307 | NZ_JHHB00000000 | Yes | | Yes | Yes |
| O127:H6 str. E2348/69 substr. CVDNalr | NZ_ASZR00000000 | | | | |
| O127:H6 str. E2348/69 substr. UMD753 | NZ_ASZS00000000 | | | | |
| O127:H27 str. C43/90 | NZ_AHAW00000000 | | | | |
| O128:H2 str. 2011C-3317 | NZ_JASU00000000 | Yes | | Yes | Yes |
| O145:H25 str. 07-3858 | NZ_JASO00000000 | Yes | | Yes | Yes |
| O145:H28 str. 2009C-3292 | NZ_JHHD00000000 | Yes | | Yes | Yes |
| O145:H28 str. 4865/96 | NZ_JHEY00000000 | Yes | Yes | | Yes |
| O145:H28 str. 4865/96 | NZ_AGTL00000000 | Yes | Yes | | Yes |
| O145:NM str. 06-3484 | NZ_JHNN00000000 | | | | |
| O145:NM str. 08-4270 | NZ_JHKV00000000 | Yes | Yes | | Yes |
| O145:NM str. 2010C-3507 | NZ_JHFW00000000 | Yes | Yes | | Yes |
| O145:NM str. 2010C-3508 | NZ_JHFV00000000 | Yes | Yes | | Yes |
| O145:NM str. 2010C-3509 | NZ_JHFU00000000 | Yes | Yes | | Yes |
| O145:NM str. 2010C-3510 | NZ_JHFT00000000 | Yes | Yes | | Yes |
| O145:NM str. 2010C-3511 | NZ_JHFS00000000 | Yes | Yes | | Yes |
| O145:NM str. 2010C-3516 | NZ_JHFR00000000 | Yes | Yes | | Yes |
| O145:NM str. 2010C-3517 | NZ_JHFQ00000000 | Yes | Yes | | Yes |
| O145:NM str. 2010C-3518 | NZ_JHFP00000000 | Yes | Yes | | Yes |
| O145:NM str. 2010C-3521 | NZ_JHFO00000000 | Yes | Yes | | Yes |
| O145:NM str. 2010C-3526 | NZ_JHFN00000000 | Yes | Yes | | Yes |
| O145:NM str. 2010C-4557C2 | NZ_JHNA00000000 | Yes | Yes | | Yes |
| O146:H21 str. 2010C-3325 | NZ_JASR00000000 | Yes | | Yes | Yes |
| O153:H2 str. 2010C-5034 | NZ_JHMH00000000 | Yes | | Yes | Yes |
| O156:H25 str. 2011C-3602 | NZ_JHLP00000000 | Yes | | Yes | Yes |
| O157:H7 | NZ_LAYW00000000 | Yes | | Yes | Yes |
| O157:H7 | NZ_LMXL00000000 | Yes | | Yes | Yes |
| O157:H7 | NZ_LCWU00000000 | Yes | | Yes | Yes |
| O157:H7 | NZ_LKAL00000000 | Yes | | Yes | Yes |
| O157:H7 | NZ_LKAK00000000 | Yes | | Yes | Yes |
| O157:H7 str. 06-3745 | NZ_JHNI00000000 | Yes | | Yes | Yes |
| O157:H7 str. 06-4039 | NZ_JHNG00000000 | Yes | | Yes | Yes |
| O157:H7 str. 07-3091 | NZ_JHNF00000000 | Yes | | Yes | Yes |
| O157:H7 str. 07-3391 | NZ_JHNE00000000 | Yes | | Yes | Yes |
| O157:H7 str. 08-3037 | NZ_JHKZ00000000 | Yes | | Yes | Yes |
| O157:H7 str. 08-3527 | NZ_JHKY00000000 | Yes | | Yes | Yes |
| O157:H7 str. 08-4169 | NZ_JHKW00000000 | Yes | | Yes | Yes |
| O157:H7 str. 08-4529 | NZ_JHHI00000000 | Yes | | Yes | Yes |
| O157:H7 str. 08BKT061141 | NZ_JJOL00000000 | | | | |
| O157:H7 str. 09BKT048303 | NZ_JJOM00000000 | | | | |
| O157:H7 str. 1044 | NZ_AERP00000000 | Yes | | Yes | Yes |
| O157:H7 str. 1125 | NZ_AERR00000000 | Yes | | Yes | Yes |
| O157:H7 str. 2009C-4258 | NZ_JHGQ00000000 | Yes | | Yes | Yes |
| O157:H7 str. 2009EL1449 | NZ_JHGF00000000 | Yes | | Yes | Yes |
| O157:H7 str. 2009EL1705 | NZ_JHGE00000000 | Yes | | Yes | Yes |
| O157:H7 str. 2009EL1913 | NZ_JHGD00000000 | Yes | | Yes | Yes |
| O157:H7 str. 2009EL2109 | NZ_JHGC00000000 | Yes | | Yes | Yes |
| O157:H7 str. 2010C-4979C1 | NZ_JHMK00000000 | Yes | | Yes | Yes |
| O157:H7 str. 2011EL-1107 | NZ_JHLK00000000 | Yes | | Yes | Yes |
| O157:H7 str. 2011EL-2090 | NZ_JHLI00000000 | Yes | | Yes | Yes |
| O157:H7 str. 2011EL-2091 | NZ_JHLH00000000 | Yes | | Yes | Yes |
| O157:H7 str. 2011EL-2092 | NZ_JHLG00000000 | Yes | | Yes | Yes |
| O157:H7 str. 2011EL-2093 | NZ_JHLF00000000 | Yes | | Yes | Yes |
| O157:H7 str. 2011EL-2094 | NZ_JHLE00000000 | Yes | | Yes | Yes |
| O157:H7 str. 2011EL-2096 | NZ_JHLD00000000 | Yes | | Yes | Yes |
| O157:H7 str. 2011EL-2097 | NZ_JHLC00000000 | Yes | | Yes | Yes |
| O157:H7 str. 2011EL-2098 | NZ_JHLB00000000 | Yes | | Yes | Yes |
| O157:H7 str. 2011EL-2099 | NZ_JHKT00000000 | Yes | | Yes | Yes |
| O157:H7 str. 2011EL-2099 | NZ_JHKT00000000 | Yes | | Yes | Yes |
| O157:H7 str. 2011EL-2101 | NZ_JHKS00000000 | Yes | | Yes | Yes |
| O157:H7 str. 2011EL-2101 | NZ_JHKS00000000 | Yes | | Yes | Yes |
| O157:H7 str. 2011EL-2103 | NZ_JHKR00000000 | Yes | | Yes | Yes |
| O157:H7 str. 2011EL-2104 | NZ_JHKQ00000000 | Yes | | Yes | Yes |
| O157:H7 str. 2011EL-2105 | NZ_JHKP00000000 | Yes | | Yes | Yes |
| O157:H7 str. 2011EL-2106 | NZ_JHKO00000000 | Yes | | Yes | Yes |
| O157:H7 str. 2011EL-2107 | NZ_JHKN00000000 | Yes | | Yes | Yes |
| O157:H7 str. 2011EL-2108 | NZ_JHKM00000000 | Yes | | Yes | Yes |
| O157:H7 str. 2011EL-2109 | NZ_JHKL00000000 | Yes | | Yes | Yes |
| O157:H7 str. 2011EL-2111 | NZ_JHKK00000000 | Yes | | Yes | Yes |
| O157:H7 str. 2011EL-2112 | NZ_JHKJ00000000 | Yes | | Yes | Yes |
| O157:H7 str. 2011EL-2113 | NZ_JHKI00000000 | Yes | | Yes | Yes |
| O157:H7 str. 2011EL-2114 | NZ_JHKH00000000 | Yes | | Yes | Yes |
| O157:H7 str. 2011EL-2286 | NZ_JHKG00000000 | Yes | | Yes | Yes |
| O157:H7 str. 2011EL-2287 | NZ_JHKF00000000 | Yes | | Yes | Yes |
| O157:H7 str. 2011EL-2288 | NZ_JHKE00000000 | Yes | | Yes | Yes |
| O157:H7 str. 2011EL-2289 | NZ_JHKD00000000 | Yes | | Yes | Yes |
| O157:H7 str. 2011EL-2290 | NZ_JHKC00000000 | Yes | | Yes | Yes |
| O157:H7 str. 2011EL-2312 | NZ_JHKB00000000 | Yes | | Yes | Yes |
| O157:H7 str. 2011EL-2313 | NZ_JHKA00000000 | Yes | | Yes | Yes |
| O157:H7 str. EC508 | NZ_ABHW00000000 | Yes | | Yes | Yes |
| O157:H7 str. EC536 | NZ_ADVC00000000 | Yes | | Yes | Yes |
| O157:H7 str. EC869 | NZ_ABHU00000000 | Yes | | Yes | Yes |
| O157:H7 str. EC1212 | NZ_AERQ00000000 | Yes | | Yes | Yes |
| O157:H7 str. EC4009 | NZ_ADMX00000000 | Yes | | Yes | Yes |
| O157:H7 str. EC4042 | NZ_ABHM00000000 | Yes | | Yes | Yes |
| O157:H7 str. EC4045 | NZ_ABHL00000000 | Yes | | Yes | Yes |
| O157:H7 str. EC4076 | NZ_ABHQ00000000 | Yes | | Yes | Yes |
| O157:H7 str. EC4113 | NZ_ABHP00000000 | Yes | | Yes | Yes |
| O157:H7 str. EC4127 | NZ_ADUZ00000000 | Yes | | Yes | Yes |
| O157:H7 str. EC4191 | NZ_ADVA00000000 | Yes | | Yes | Yes |
| O157:H7 str. EC4192 | NZ_ADUX00000000 | Yes | | Yes | Yes |
| O157:H7 str. EC4196 | NZ_ABHO00000000 | Yes | | Yes | Yes |
| O157:H7 str. EC4206 | NZ_ABHK00000000 | Yes | | Yes | Yes |
| O157:H7 str. EC4401 | NZ_ABHR00000000 | Yes | | Yes | Yes |
| O157:H7 str. EC4486 | NZ_ABHS00000000 | Yes | | Yes | Yes |
| O157:H7 str. EC4501 | NZ_ABHT00000000 | Yes | | Yes | Yes |
| O157:H7 str. F6142 | NZ_JHJT00000000 | Yes | | Yes | Yes |
| O157:H7 str. F6749 | NZ_JHJQ00000000 | Yes | | Yes | Yes |
| O157:H7 str. F6750 | NZ_JHJP00000000 | Yes | | Yes | Yes |
| O157:H7 str. F6751 | NZ_JHJO00000000 | Yes | | Yes | Yes |
| O157:H7 str. F7350 | NZ_JHJN00000000 | Yes | | Yes | Yes |
| O157:H7 str. F7377 | NZ_JHJM00000000 | Yes | | Yes | Yes |
| O157:H7 str. F7384 | NZ_JHJL00000000 | Yes | | Yes | Yes |
| O157:H7 str. F7410 | NZ_JHJK00000000 | Yes | | Yes | Yes |
| O157:H7 str. K1420 | NZ_JHJF00000000 | Yes | | Yes | Yes |
| O157:H7 str. K1792 | NZ_JHJD00000000 | Yes | | Yes | Yes |
| O157:H7 str. K1793 | NZ_JHJC00000000 | Yes | | Yes | Yes |
| O157:H7 str. K1795 | NZ_JHJB00000000 | Yes | | Yes | Yes |
| O157:H7 str. K1796 | NZ_JHJA00000000 | Yes | | Yes | Yes |
| O157:H7 str. K1845 | NZ_JHIZ00000000 | Yes | | Yes | Yes |
| O157:H7 str. K1921 | NZ_JHIY00000000 | Yes | | Yes | Yes |
| O157:H7 str. K1927 | NZ_JHIX00000000 | Yes | | Yes | Yes |
| O157:H7 str. K2188 | NZ_JHIW00000000 | Yes | | Yes | Yes |
| O157:H7 str. K2191 | NZ_JHIV00000000 | Yes | | Yes | Yes |
| O157:H7 str. K2192 | NZ_JHIU00000000 | Yes | | Yes | Yes |
| O157:H7 str. K2324 | NZ_JHIT00000000 | Yes | | Yes | Yes |
| O157:H7 str. K2581 | NZ_JHIS00000000 | Yes | | Yes | Yes |
| O157:H7 str. K2622 | NZ_JHIR00000000 | Yes | | Yes | Yes |
| O157:H7 str. K2845 | NZ_JHIQ00000000 | Yes | | Yes | Yes |
| O157:H7 str. K2854 | NZ_JHIP00000000 | Yes | | Yes | Yes |
| O157:H7 str. K4396 | NZ_JHIO00000000 | Yes | | Yes | Yes |
| O157:H7 str. LSU-61 | NZ_AEUC00000000 | Yes | | Yes | Yes |
| O157:H7 str. TW14313 | NZ_AKMD00000000 | | | | |
| O157:H43 str. T22 | NZ_AHZD00000000 | Yes | | Yes | Yes |
| O157:H− str. 493-89 | NZ_AETY00000000 | Yes | | Yes | Yes |
| O157:H− str. 493-89 | NZ_AGTG00000000 | Yes | | Yes | Yes |
| O157:H− str. H 2687 | NZ_AETZ00000000 | Yes | | Yes | Yes |
| O157:NM str. 08-4540 | NZ_JHHH00000000 | Yes | | Yes | Yes |
| O157: str. 2010EL-2044 | NZ_JHME00000000 | Yes | | Yes | Yes |
| O157: str. 2010EL-2045 | NZ_JHMD00000000 | Yes | | Yes | Yes |
| O157 str. NCCP15738 | NZ_ASHB00000000 | | | | |
| O157 str. NCCP15739 | NZ_ASHA00000000 | Yes | | Yes | Yes |
| STEC_7v | NZ_AEXD00000000 | | | | |
| STEC_B2F1 | NZ_AFDQ00000000 | Yes | | Yes | Yes |
| STEC_C165-02 | NZ_AFDR00000000 | Yes | | Yes | |
| STEC_EH250 | NZ_AFDW00000000 | Yes | | Yes | |
| STEC_MHI813 | NZ_AFDZ00000000 | Yes | | Yes | Yes |
| STEC_O31 | NZ_AFEX00000000 | Yes | | Yes | Yes |
| STEC_S1191 | NZ_AFEA00000000 | Yes | | Yes | |
| STEC O174:H8 str. 02-07607 | NZ_AQGN00000000 | Yes | | Yes | |
| STEC 29 | NZ_LNFU00000000 | Yes | | Yes | |
| STEC 66 | NZ_LNFT00000000 | Yes | | Yes | |
| STEC 168 | NZ_LNFV00000000 | Yes | | Yes | |
| STEC 169 | NZ_LNZJ00000000 | | | | |
| STEC 196 | NZ_LNZK00000000 | Yes | | Yes | |
| STEC 200 | NZ_LNZL00000000 | Yes | | Yes | Yes |
| STEC 299 | NZ_LOCR00000000 | Yes | | Yes | Yes |
| STEC 309 | NZ_LOCS00000000 | Yes | | Yes | Yes |
| STEC 329 | NZ_LOCT00000000 | Yes | | Yes | |
| STEC 343 | NZ_LDOZ00000000 | Yes | | Yes | Yes |
| STEC 370 | NZ_LOCU00000000 | Yes | | Yes | Yes |
| STEC 380 | NZ_LOCV00000000 | Yes | | Yes | Yes |
| STEC 384 | NZ_LOCW00000000 | Yes | | Yes | Yes |
| STEC 464 | NZ_LOCX00000000 | Yes | | Yes | Yes |
| STEC 477 | NZ_LOCY00000000 | Yes | | Yes | Yes |
| STEC 479 | NZ_LOCZ00000000 | Yes | | Yes | Yes |
| STEC 487 | NZ_LODA00000000 | Yes | | Yes | Yes |
| STEC 545 | NZ_LODB00000000 | Yes | Yes | | Yes |
| STEC 559 | NZ_LODC00000000 | Yes | | Yes | Yes |
| STEC 563 | NZ_LODD00000000 | Yes | | Yes | Yes |
| STEC 565 | NZ_LODE00000000 | | | | |
| STEC 605 | NZ_LFUA00000000 | Yes | | Yes | Yes |
| STEC 623 | NZ_LFUB00000000 | Yes | | Yes | Yes |
| STEC 627 | NZ_LODF00000000 | Yes | | Yes | Yes |
| STEC 645 | NZ_LODG00000000 | Yes | | Yes | |
| STEC 690 | NZ_LOFJ00000000 | | | | |
| STEC 691 | NZ_LOFK00000000 | Yes | | Yes | Yes |
| STEC 707 | NZ_LOFL00000000 | Yes | | Yes | Yes |
| STEC 709 | NZ_LOFM00000000 | Yes | | Yes | Yes |
| STEC 731 | NZ_LOFN00000000 | Yes | Yes | | Yes |
| STEC 757 | NZ_LOFO00000000 | Yes | | Yes | Yes |
| STEC 764 | NZ_LOFP00000000 | Yes | | Yes | Yes |
| STEC 793 | NZ_LOFQ00000000 | | | | |
| STEC 886 | NZ_LOFR00000000 | Yes | | Yes | Yes |
| STEC 931 | NZ_LOFS00000000 | Yes | | Yes | Yes |
| STEC 940 | NZ_LOFT00000000 | | | | |
| STEC 1117 | NZ_LOFU00000000 | Yes | | Yes | Yes |
| STEC 1161 | NZ_LOFV00000000 | Yes | | Yes | Yes |
| STEC 1178 | NZ_LOFW00000000 | | | | |
| STEC 1188 | NZ_LOFX00000000 | Yes | | Yes | Yes |
| STEC 1198 | NZ_LOFY00000000 | | | | |
| STEC 1201 | NZ_LOFZ00000000 | Yes | | Yes | Yes |
| STEC 1225 | NZ_LOGA00000000 | Yes | | Yes | Yes |
| STEC 1236 | NZ_LOGB00000000 | Yes | | Yes | Yes |
| STEC 1255 | NZ_LOGC00000000 | Yes | | Yes | Yes |
| STEC 1270 | NZ_LOGD00000000 | | | | |
| STEC 1284 | NZ_LOGE00000000 | Yes | | Yes | Yes |
| STEC 1293 | NZ_LOGF00000000 | Yes | | Yes | Yes |
| STEC 1299 | NZ_LOGG00000000 | Yes | | Yes | Yes |
| STEC 1303 | NZ_LOGH00000000 | | | | |
| STEC 1363 | NZ_LOGI00000000 | Yes | | Yes | Yes |
| STEC 1375 | NZ_LOGJ00000000 | Yes | | Yes | Yes |
| STEC 1442 | NZ_LOGK00000000 | Yes | Yes | | Yes |
| STEC 1465 | NZ_LOGL00000000 | Yes | | Yes | Yes |
| STEC 1473 | NZ_LOGM00000000 | | | | |
| STEC 1500 | NZ_LOGN00000000 | Yes | | Yes | |
| STEC 1513 | NZ_LOGO00000000 | Yes | | Yes | Yes |
| STEC 1528 | NZ_LOGP00000000 | | | | |
| STEC 1532 | NZ_LOGQ00000000 | Yes | | Yes | Yes |
| STEC 1585 | NZ_LOGR00000000 | Yes | | Yes | |
| STEC 1634 | NZ_LOGS00000000 | Yes | | Yes | Yes |
| STEC 1686 | NZ_LOGT00000000 | Yes | | Yes | Yes |
| STEC 2064 | NZ_LOJC00000000 | Yes | | Yes | |
| STEC 2074 | NZ_LOJD00000000 | Yes | | Yes | Yes |
| STEC 2075 | NZ_LGBD00000000 | Yes | | Yes | Yes |
| STEC 2110.1 | NZ_LPWW00000000 | Yes | | Yes | |
| STEC 2110.3 | NZ_LOJE00000000 | Yes | | Yes | Yes |
| STEC 2112 | NZ_LGBE00000000 | Yes | | Yes | Yes |
| STEC 2144 | NZ_LOGU00000000 | Yes | | Yes | Yes |
| STEC 2174 | NZ_LOGV00000000 | Yes | | Yes | |
| STEC 2193 | NZ_LOGW00000000 | Yes | | Yes | Yes |
| STEC 2211 | NZ_LOGX00000000 | Yes | | Yes | Yes |
| STEC 2236 | NZ_LOGY00000000 | | | | |
| STEC 2257 | NZ_LGBF00000000 | Yes | | Yes | Yes |
| STEC 2270 | NZ_LPWX00000000 | Yes | | Yes | Yes |
| STEC 2334 | NZ_LOGZ00000000 | | | | |
| STEC 2346 | NZ_LOHA00000000 | | | | |
| STEC 2359 | NZ_LOHB00000000 | Yes | | Yes | Yes |
| STEC 2363 | NZ_LPWY00000000 | Yes | | Yes | |
| STEC 2410 | NZ_LGBG00000000 | Yes | | Yes | Yes |
| STEC 2419 | NZ_LPWZ00000000 | | | | |
| STEC 2441 | NZ_LOHC00000000 | Yes | | Yes | Yes |
| STEC 2450 | NZ_LPXA00000000 | Yes | | Yes | Yes |
| STEC 2499 | NZ_LOIE00000000 | Yes | | Yes | Yes |
| STEC 2505 | NZ_LPXB00000000 | Yes | | Yes | Yes |
| STEC 2539 | NZ_LOIF00000000 | | | | |
| STEC 2564 | NZ_LOIG00000000 | | | | |
| STEC 2573 | NZ_LOIH00000000 | Yes | | Yes | Yes |
| STEC 2591 | NZ_LOII00000000 | Yes | | Yes | Yes |
| STEC 2595 | NZ_LOIJ00000000 | Yes | | Yes | |
| STEC 2620 | NZ_LPXC00000000 | Yes | | Yes | |
| STEC 2633 | NZ_LOIK00000000 | | | | |
| STEC 2667 | NZ_LGBH00000000 | Yes | | Yes | Yes |
| STEC 2708 | NZ_LOIL00000000 | | | | |
| STEC 2743 | NZ_LOIM00000000 | Yes | | Yes | Yes |
| STEC 2746 | NZ_LPXD00000000 | Yes | | Yes | Yes |
| STEC 2764 | NZ_LOIN00000000 | Yes | | Yes | |
| STEC 2770 | NZ_LPXF00000000 | Yes | | Yes | Yes |
| STEC 2788 | NZ_LOIO00000000 | Yes | | Yes | |
| STEC 2797 | NZ_LOIP00000000 | Yes | | Yes | |
| STEC 2820 | NZ_LGBQ00000000 | Yes | | Yes | Yes |
| STEC 2821 | NZ_LGBI00000000 | Yes | | Yes | Yes |
| STEC 2826 | NZ_LOJA00000000 | Yes | | Yes | Yes |
| STEC 2839 | NZ_LOJB00000000 | Yes | | Yes | |
| STEC 2841 | NZ_LOIQ00000000 | Yes | | Yes | Yes |
| STEC 2861 | NZ_LOIR00000000 | Yes | | Yes | |
| STEC 2868 | NZ_LGBJ00000000 | Yes | | Yes | Yes |
| STEC 2894.1 | NZ_LOIS00000000 | Yes | | Yes | |
| STEC 2894.2 | NZ_LOIT00000000 | | | | |
| STEC 2920 | NZ_LOIU00000000 | Yes | | Yes | Yes |
| STEC 2938 | NZ_LOIV00000000 | Yes | | Yes | Yes |
| STEC 2953 | NZ_LOIW00000000 | | | | |
| STEC 2954 | NZ_LPXE00000000 | Yes | | Yes | |
| STEC 2962 | NZ_LOIX00000000 | Yes | | Yes | |
| STEC 2980 | NZ_LOIY00000000 | Yes | | Yes | Yes |
| STEC 3031 | NZ_LOIZ00000000 | | | | |
| STEC 3039 | NZ_LPUH00000000 | Yes | | Yes | Yes |
| STEC 3055 | NZ_LPUI00000000 | Yes | | Yes | |
| STEC 3084 | NZ_LPUJ00000000 | Yes | | Yes | |
| STEC 3087 | NZ_LPUK00000000 | Yes | | Yes | |
| STEC 3094 | NZ_LPUL00000000 | Yes | | Yes | Yes |
| STEC 3098 | NZ_LPUM00000000 | Yes | | Yes | Yes |
| STEC 3106 | NZ_LPUN00000000 | Yes | | Yes | |
| upec-2 | NZ_JSLN00000000 | Yes | Yes | | Yes |
| upec-3 | NZ_JSIF00000000 | Yes | | Yes | Yes |
| upec-4 | NZ_JSHU00000000 | | | | |
| upec-7 | NZ_JSGV00000000 | | | | |
| upec-8 | NZ_JSGK00000000 | | | | |
| upec-9 | NZ_JSFZ00000000 | | | | |
| upec-10 | NZ_JSPB00000000 | | | | |
| upec-15 | NZ_JSNA00000000 | | | | |
| upec-22 | NZ_JSKT00000000 | | | | |
| upec-23 | NZ_JSKJ00000000 | | | | |
| upec-24 | NZ_JSJZ00000000 | | | | |
| upec-28 | NZ_JSIQ00000000 | | | | |
| upec-29 | NZ_JSIG00000000 | | | | |
| upec-30 | NZ_JSIE00000000 | Yes | | Yes | Yes |
| upec-31 | NZ_JSID00000000 | Yes | | Yes | Yes |
| upec-33 | NZ_JSIB00000000 | Yes | | Yes | |
| upec-34 | NZ_JSIA00000000 | | | | |
| upec-36 | NZ_JSHY00000000 | | | | |
| upec-37 | NZ_JSHX00000000 | | | | |
| upec-38 | NZ_JSHW00000000 | | | | |
| upec-39 | NZ_JSHV00000000 | | | | |
| upec-50 | NZ_JSHM00000000 | | | | |
| upec-51 | NZ_JSHL00000000 | | | | |
| upec-53 | NZ_JSHK00000000 | | | | |
| upec-54 | NZ_JSHJ00000000 | | | | |
| upec-55 | NZ_JSHI00000000 | | | | |
| upec-56 | NZ_JSHH00000000 | Yes | | Yes | |
| upec-57 | NZ_JSHG00000000 | | | | |
| upec-58 | NZ_JSHF00000000 | | | | |
| upec-59 | NZ_JSHE00000000 | | | | |
| upec-60 | NZ_JSHD00000000 | | | | |
| upec-61 | NZ_JSHC00000000 | | | | |
| upec-62 | NZ_JSHB00000000 | | | | |
| upec-64 | NZ_JSHA00000000 | | | | |
| upec-65 | NZ_JSGZ00000000 | | | | |
| upec-66 | NZ_JSGY00000000 | Yes | | Yes | Yes |
| upec-69 | NZ_JSGW00000000 | Yes | | Yes | |
| upec-70 | NZ_JSGU00000000 | | | | |
| upec-72 | NZ_JSGS00000000 | | | | |
| upec-73 | NZ_JSGRO0000000 | | | | |
| upec-74 | NZ_JSGQ00000000 | | | | |
| upec-75 | NZ_JSGP00000000 | | | | |
| upec-76 | NZ_JSGO00000000 | | | | |
| upec-77 | NZ_JSGN00000000 | | | | |
| upec-78 | NZ_JSGM00000000 | | | | |
| upec-79 | NZ_JSGL00000000 | | | | |
| upec-80 | NZ_JSGJ00000000 | | | | |
| upec-81 | NZ_JSGI00000000 | Yes | | Yes | Yes |
| upec-82 | NZ_JSGH00000000 | Yes | | Yes | |
| upec-83 | NZ_JSGG00000000 | | | | |
| upec-84 | NZ_JSGF00000000 | | | | |
| upec-85 | NZ_JSGE00000000 | | | | |
| upec-87 | NZ_JSGC00000000 | | | | |
| upec-88 | NZ_JSGB00000000 | | | | |
| upec-89 | NZ_JSGA00000000 | | | | |
| upec-90 | NZ_JSFY00000000 | | | | |
| upec-91 | NZ_JSFX00000000 | | | | |
| upec-93 | NZ_JSFV00000000 | | | | |
| upec-94 | NZ_JSFU00000000 | | | | |
| upec-95 | NZ_JSFT00000000 | Yes | | Yes | Yes |
| upec-97 | NZ_JSFS00000000 | | | | |
| upec-98 | NZ_JSFR00000000 | | | | |
| upec-99 | NZ_JSFQ00000000 | Yes | | Yes | |
| upec-100 | NZ_JSPA00000000 | | | | |
| upec-101 | NZ_JSOZ00000000 | | | | |
| upec-103 | NZ_JSOX00000000 | | | | |
| upec-104 | NZ_JSOW00000000 | Yes | | Yes | Yes |
| upec-105 | NZ_JSOV00000000 | | | | |
| upec-106 | NZ_JSOU00000000 | | | | |
| upec-107 | NZ_JSOT00000000 | | | | |
| upec-108 | NZ_JSOS00000000 | | | | |
| upec-109 | NZ_JSOR00000000 | | | | |
| upec-110 | NZ_JSOQ00000000 | | | | |
| upec-114 | NZ_JSOM00000000 | Yes | | Yes | Yes |
| upec-115 | NZ_JSOL00000000 | | | | |
| upec-116 | NZ_JSOK00000000 | | | | |
| upec-117 | NZ_JSOJ00000000 | | | | |
| upec-118 | NZ_JSOI00000000 | Yes | | Yes | Yes |
| upec-119 | NZ_JSOH00000000 | Yes | | Yes | Yes |
| upec-120 | NZ_JSOF00000000 | | | | |
| upec-121 | NZ_JSOE00000000 | Yes | | Yes | Yes |
| upec-123 | NZ_JSOC00000000 | | | | |
| upec-124 | NZ_JSOB00000000 | | | | |
| upec-125 | NZ_JSOA00000000 | | | | |
| upec-126 | NZ_JSNZ00000000 | | | | |
| upec-127 | NZ_JSNY00000000 | | | | |
| upec-128 | NZ_JSNX00000000 | | | | |
| upec-129 | NZ_JSNW00000000 | | | | |
| upec-130 | NZ_JSNV00000000 | Yes | | Yes | Yes |
| upec-131 | NZ_JSNU00000000 | | | | |
| upec-132 | NZ_JSNT00000000 | Yes | | Yes | Yes |
| upec-133 | NZ_JSNS00000000 | Yes | | Yes | |
| upec-134 | NZ_JSNR00000000 | | | | |
| upec-135 | NZ_JSNQ00000000 | | | | |
| upec-136 | NZ_JSNP00000000 | | | | |
| upec-137 | NZ_JS00000000 | Yes | | Yes | Yes |
| upec-138 | NZ_JSNN00000000 | | | | |
| upec-139 | NZ_JSNM00000000 | | | | |
| upec-140 | NZ_JSNK00000000 | | | | |
| upec-141 | NZ_JSNJ00000000 | | | | |
| upec-142 | NZ_JSNI00000000 | | | | |
| upec-143 | NZ_JSNH00000000 | | | | |
| upec-144 | NZ_JSNG00000000 | | | | |
| upec-145 | NZ_JSNF00000000 | Yes | | Yes | |
| upec-146 | NZ_JSNE00000000 | Yes | | Yes | Yes |
| upec-147 | NZ_JSND00000000 | Yes | | Yes | Yes |
| upec-148 | NZ_JSNC00000000 | | | | |
| upec-149 | NZ_JSNB00000000 | | | | |
| upec-150 | NZ_JSMZ00000000 | Yes | | Yes | Yes |
| upec-151 | NZ_JSMY00000000 | Yes | | Yes | Yes |
| upec-153 | NZ_JSMX00000000 | | | | |
| upec-154 | NZ_JSMW00000000 | | | | |
| upec-155 | NZ_JSMV00000000 | Yes | | Yes | |
| upec-156 | NZ_JSMU00000000 | | | | |
| upec-157 | NZ_JSMT00000000 | | | | |
| upec-158 | NZ_JSMS00000000 | | | | |
| upec-159 | NZ_JSMR00000000 | | | | |
| upec-161 | NZ_JSMQ00000000 | | | | |
| upec-162 | NZ_JSMP00000000 | | | | |
| upec-166 | NZ_JSMO00000000 | | | | |
| upec-169 | NZ_JSMN00000000 | | | | |
| upec-170 | NZ_JSML00000000 | Yes | Yes | | Yes |
| upec-171 | NZ_JSMK00000000 | | | | |
| upec-172 | NZ_JSMJ00000000 | | | | |
| upec-173 | NZ_JSMI00000000 | Yes | Yes | | Yes |
| upec-175 | NZ_JSMH00000000 | Yes | | Yes | Yes |
| upec-176 | NZ_JSMG00000000 | | | | |
| upec-207 | NZ_JSLE00000000 | | | | |
| upec-208 | NZ_JSLD00000000 | | | | |
| upec-209 | NZ_JSLC00000000 | | | | |
| upec-211 | NZ_JSLB00000000 | Yes | | Yes | Yes |
| upec-212 | NZ_JSLA00000000 | | | | |
| upec-213 | NZ_JSKZ00000000 | Yes | | Yes | Yes |
| upec-219 | NZ_JSKU00000000 | | | | |
| upec-220 | NZ_JSKS00000000 | | | | |
| upec-221 | NZ_JSKR00000000 | Yes | | Yes | |
| upec-225 | NZ_JSKO00000000 | | | | |
| upec-226 | NZ_JSKN00000000 | | | | |
| upec-227 | NZ_JSKM00000000 | | | | |
| upec-228 | NZ_JSKL00000000 | | | | |
| upec-229 | NZ_JSKK00000000 | | | | |
| upec-230 | NZ_JSKI00000000 | | | | |
| upec-232 | NZ_JSKG00000000 | | | | |
| upec-233 | NZ_JSKF00000000 | | | | |
| upec-235 | NZ_JSKE00000000 | | | | |
| upec-236 | NZ_JSKD00000000 | | | | |
| upec-237 | NZ_JSKC00000000 | | | | |
| upec-238 | NZ_JSKB00000000 | Yes | | Yes | Yes |
| upec-239 | NZ_JSKA00000000 | | | | |
| upec-243 | NZ_JSJX00000000 | | | | |
| upec-244 | NZ_JSJW00000000 | | | | |
| upec-248 | NZ_JSJT00000000 | | | | |
| upec-249 | NZ_JSJS00000000 | | | | |
| upec-250 | NZ_JSJQ00000000 | | | | |
| upec-251 | NZ_JSJP00000000 | | | | |
| upec-253 | NZ_JSJO00000000 | | | | |
| upec-254 | NZ_JSJN00000000 | Yes | | Yes | |
| upec-255 | NZ_JSJM00000000 | | | | |
| upec-256 | NZ_JSJL00000000 | Yes | | Yes | |
| upec-257 | NZ_JSJK00000000 | | | | |
| upec-258 | NZ_JSJJ00000000 | | | | |
| upec-259 | NZ_JSJI00000000 | | | | |
| upec-260 | NZ_JSJG00000000 | | | | |
| upec-261 | NZ_JSJF00000000 | | | | |
| upec-265 | NZ_JSJB00000000 | | | | |
| upec-266 | NZ_JSJA00000000 | | | | |
| upec-269 | NZ_JSIY00000000 | Yes | Yes | | Yes |
| upec-271 | NZ_JSIV00000000 | | | | |
| upec-273 | NZ_JSIU00000000 | Yes | Yes | | Yes |
| upec-274 | NZ_JSIT00000000 | Yes | | Yes | |
| upec-276 | NZ_JSIS00000000 | | | | |
| upec-277 | NZ_JSIR00000000 | | | | |
| upec-281 | NZ_JSIO00000000 | | | | |
| upec-282 | NZ_JSIN00000000 | | | | |
| upec-284 | NZ_JSIM00000000 | | | | |
| upec-285 | NZ_JSIL00000000 | | | | |
| upec-286 | NZ_JSIK00000000 | | | | |
| upec-287 | NZ_JSIJ00000000 | | | | |
| upec-288 | NZ_JSII00000000 | | | | |
| upec-289 | NZ_JSIH00000000 | | | | |
| Totals | 628 | 401 | 25 | 376 | 355 |
| Percentages | | 63.85 | 6.23 | 93.77 | 88.53 |

### Example 14: Expression of LeuO is Necessary to Elicit CRISPR-Cas Lethality

Most E. coli encode the necessary components for Type I CRISPR-Cas3 activity in their genome. However, the E. coli Type I-E CRISPR-Cas3 operon is regulated by histone-like nucleoid-structuring (H-NS) repression and is not expressed under normal culture conditions. LeuO acts in opposition to H-NS at overlapping promoter regions and activates gene expression. The interplay between H-NS and LeuO activity has been studied in S. typhimurium and E. coli, by examining the global transcriptional changes related to LeuO overexpression or knockout. Under conventional culturing conditions, LeuO itself is not expressed but is upregulated during starvation and stationary phase. However, the casABCDE operon in E. coli and S. typhimurium was significantly upregulated with LeuO overexpression and has predicted H-NS and LeuO binding sequences upstream of CasA. However, in the absence of a LeuO expression cassette, casABCDE expression is not sufficient to support lethality via self-targeting crRNAs.

The primary risks of delivering a LeuO expression cassette to pathogens include its impact on the non-target (non-Cas) genes within its regulon. In S. typhimurium, LeuO upregulates some genes related to pathogenicity, but it is unclear if there is a meaningful increase in expression and how this observation applies to bacteria other than S. typhimurium. In E. coli, LeuO increases or decreases resistance to certain classes of antibiotics.

To verify functionality of LeuO in CRISPR-mediated lethality in E. coli, a phagemid was designed encoding a LeuO expression cassette to overcome the wild-type repression of the endogenous CRISPR-Cas3 operon. The designed phagemid was derived from the M13 bacteriophage, which has been shown to be non-lytic so as not to confound CRISPR-Cas3 based lethality. The phagemid also encodes a CRISPR array targeting the conserved E. coli ftsA gene, whereby expression of this array activates and direct self-targeting of Type I-E E. coli CRISPR-Cas3 systems to elicit cell death.

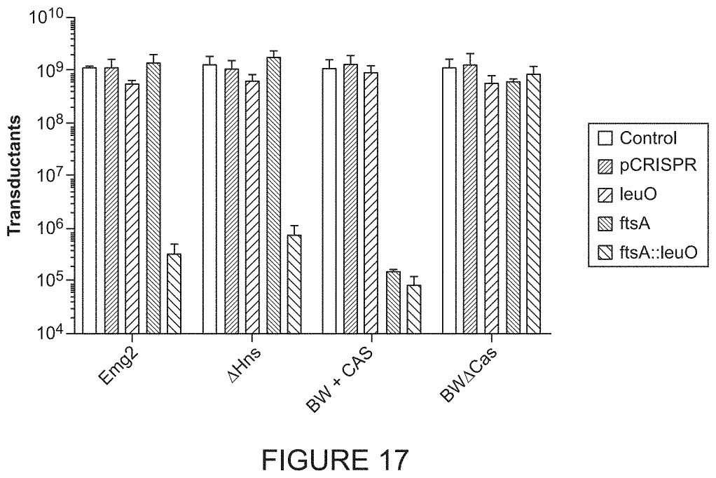

FIG. 17 shows non-lytic M13-derived phagemid delivery of CRISPR constructs using the validated ftsA spacer sequence designed to test the dependence on LeuO expression for CRISPR-mediated lethality. Phagemids were produced to titers of 10⁹ transducing units per milliliter and maintained in growth media for in vitro studies. The phagemid vector encodes the ftsA repeat-spacer array, LeuO expression cassette, and an M13-compatible origin of replication. Lethality of phagemid vectors was tested via transduction of M13 bacteriophages into a range of s including a parent EMG2 containing a wild-type H-NS repressed E. coli Type I-E CRISPR-Cas3 operon, a BW25113-derivative lacking the H-NS repression motifs in the CRISPR-Cas3 operon (Δhns), a BW25113-derivative containing an overexpressed CRISPR-Cas3 operon (BW+Cas) and a BW25113-derivative lacking Cas3 genes (BWΔCas).

Indicated E. coli were infected with 10⁹ transducing units per milliliter of each M13 phagemid and plated on selective media to recover transduced cells and count surviving colony forming units (transductants). Each was transduced with the following phagemids indicated in the legend: Control, generic M13 transduction control; pCRISPR, phagemid that constitutively expresses non-targeting crRNA; LeuO, phagemid that constitutively expresses the E. coli LeuO gene; ftsA, phagemid that constitutively expresses crRNA targeting conserved ftsA gene present in E. coli; ftsA::LeuO, phagemid constitutively expresses LeuO gene and crRNA targeting ftsA.

Co-delivery of LeuO and the ftsA-targeting spacer resulted in reductions in the range of 3.4-log (±0.04) to 4.3-log (±0.06) compared to control across each except the BWΔCas that lacks Cas3 activity, confirming that lethality is dependent on the constructs expressed from the phagemid genome. Notably, CRISPR-Cas3 lethality by expression of a ftsA-targeting spacer alone was only observed in the BW+Cas cell line, demonstrating that removal of H-NS repression alone is not sufficient to rescue significant levels of endogenous CRISPR-Cas3 targeting. Based on these data, both the ftsA spacer and LeuO are required for cell death in non-engineered, wild-type E. coli.

### Example 15: LeuO Enhanced crPhages have Improved Lethality Kinetics

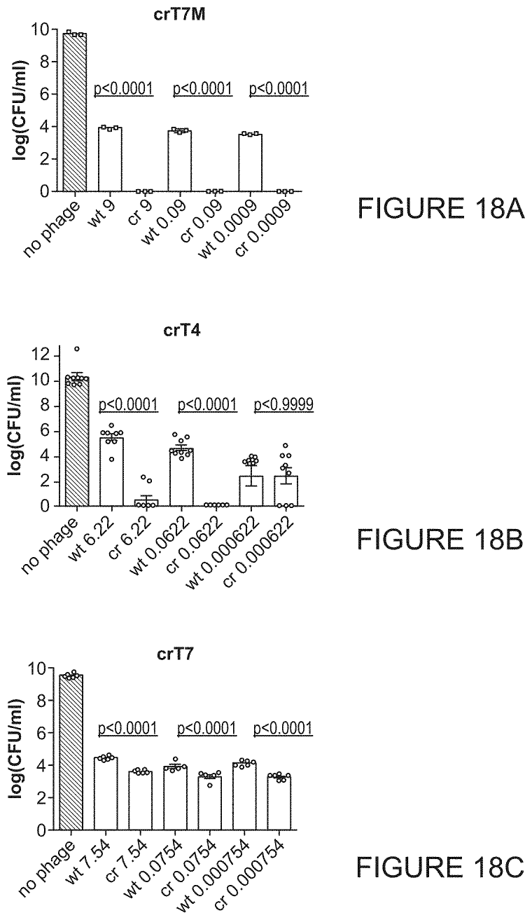

Each crPhage was systemically compared to wild-type phage to determine change in potency of CRISPR-enhanced phages compared to their respective wild-type bacteriophage. crPhages and the corresponding wild-type bacteriophage were produced, filtered, and adjusted to the same titer in growth media. Based on existing publicly available sequencing data, the three wild-type phages have significantly different genomic architectures, but are obligate lytic phages (data not shown). Target E. coli were incubated for 2 or 5 hours for crT7m, crT7 and crT4, respectively, in growth media at the indicated multiplicity-of-infection (ratio of phage to bacteria) for each phage. After incubation, cultures were immediately collected, serially diluted and plated to count surviving colonies. Significant differences were observed in CFU reduction across all three crPhage: wild-type phage comparisons as seen in FIG. 18A-FIG. 18C, including up to an approximately 4-log improvement of crT7m (FIG. 18A), approximately 4.5-log improvement of crT4 (FIG. 18B) and approximately 1-log improvement of crT7 (FIG. 18C) activities. These data suggest that CRISPR-enhanced phages eliminate the target E. coli population, in contrast to the wild-type bacteriophage, at the selected time points.

### Example 16: LeuO Enhanced crPhages In Vitro Kill Curves

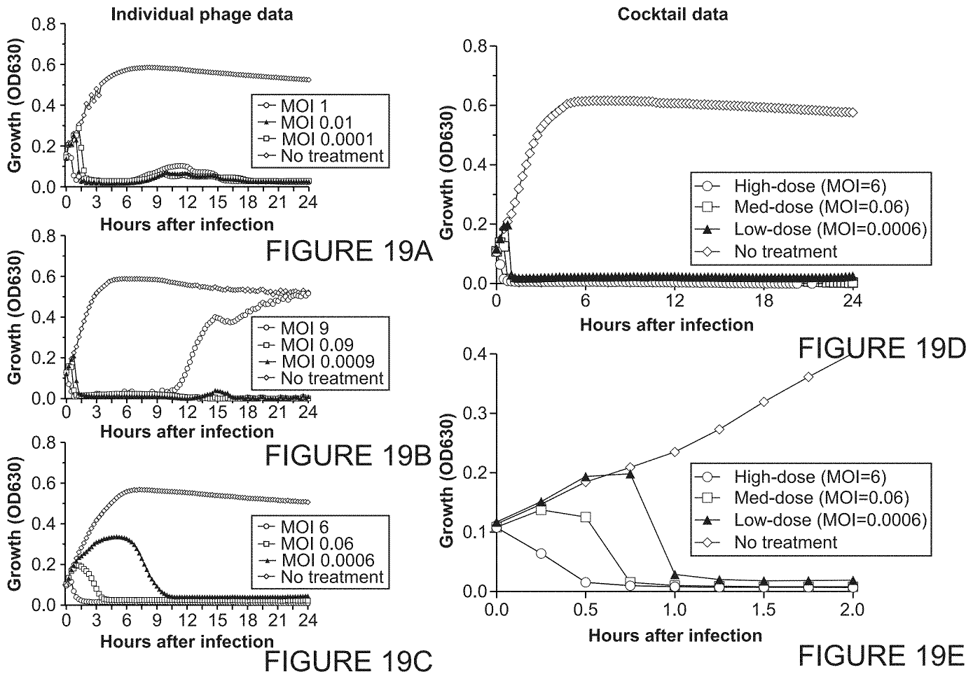

FIG. 19A-FIG. 19E shows the dose-response in vitro kill curves for each crPhage. Each crPhage was produced by standard lytic amplification; filtration and left suspended in the original growth media (LB broth). All experiments were conducted in LB broth. E. coli MG1655 was grown to mid-log phase and then mixed with the indicated multiplicity-of-infection (MOI) of each crPhage, crPhage cocktail or LB only negative control. Treated populations were grown under aerobic, shaking conditions at 37 C for 24 hours in a plate reader to monitor growth of treated populations by optical density (OD 630 nm).

E. coli MG1655 was grown to mid-log phase and treated with multiplicity-of-infection (MOI; ratio of phage to bacteria) as follows: FIG. 19A, crT7 was incubated at MOIs of 0.0001, 0.01, and 1.0; FIG. 19B, crT7m was incubated at MOIs of 0.0009, 0.09, and 9.0; and FIG. 19C, crT4 was incubated at MOIs of 0.0006, 0.06, and 6.0. Each phage was mixed in equal amounts to create a crPhage cocktail (‘Cocktail’) and was incubated at MOIs (for each crPhage) of 0.0006, 0.06, and 6.0 as seen in FIG. 19D. FIG. 19E is a zoomed in graph from FIG. 19D.

As expected, all crPhages lysed the target independent of MOI. Notably, an emergent resistant population in the crT7m high-dose population (FIG. 19B) and in the crT4 low-dose population (MOI=0.0006, FIG. 19C) was observed by 24 hours of continuous culture in the presence of the initial phage dose. However, when challenged with a cocktail of crT7, crT7m and crT4, no resistant population was observed (FIG. 19D and FIG. 19E).

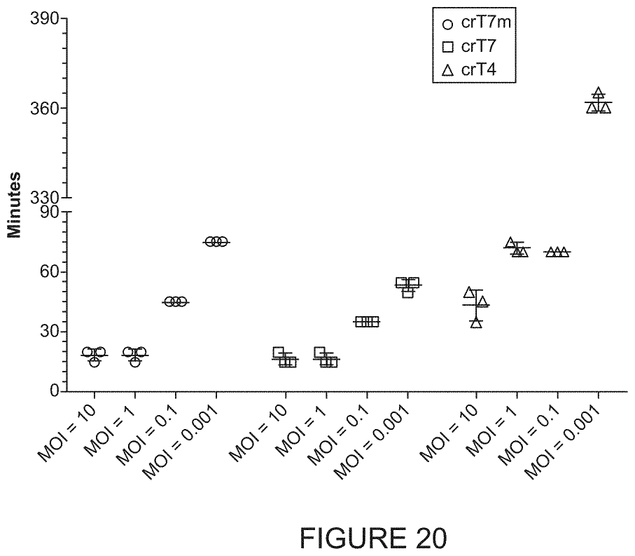

A dose-dependent relationship was observed between concentrations of crPhage and observable time-to-lysis as seen in FIG. 20 conducted to quantify time-to-lysis compared to MOI. Time-to-lysis was defined as the time at which the first derivative of the growth curve reaches zero after the bacterial population crashes due to presumed lytic phage amplification. Endonuclease degradation of host genomes is considered to be a non-lytic mechanism for killing bacteria, thus CRISPR-mediated lethality is not expected to be observed by growth curve analysis.

E. coli MG1655 was grown to mid-log phase and treated with multiplicity-of-infection (MOI; ratio of phage to bacteria) as indicated for each crPhage. Growth curves were smoothed and the first derivative of the smoothed lines were determined using the PRISM software suite. Time-to-lysis was calculated as the time where the first derivative reaches zero immediately following the initial observed population decline.

For all 3 crPhages tested, MOI in excess of 1.0 result in fastest time-to-lysis, presumably being limited by the lytic period of each phage. The observed time-to-lysis of approximately 15-20 minutes for crT7m and crT7 and 45-50 minutes for crT4 largely agree with the values known for the wild-type lytic phages T7 (˜17 minutes), T7m (˜15-20 minutes) and T4 (35 minutes), respectively.

### Example 17: LeuO Enhanced crPhages In Vivo Tolerability

Prior to survival studies in the peritonitis model, the tolerability of each crPhage was determined in vivo. crPhages were prepared for tolerability as described in Table 13 below.

TABLE 13
crPhages for in vivo tolerability studies.

| | | Batch information | | | | | Dosing information | | |
|---|---|---|---|---|---|---|---|---|---|
| Study information | | | | Production | | | Dose | # | |
| Study | Study type | Phage | Diluent | notes | PFU/mL | EU/mL | (mL) | doses | RoA |
| 035467 | Tolerability | crT7 | 0.9% saline | CF; TP | 2.0E+12 | 3 | 0.1 | 5 | i.p. |
| 035467 | Tolerability | Control | 0.9% saline | | N/A | <1 | 0.1 | 5 | i.p. |
| 035551 | Tolerability | crT7m | 0.9% saline | CF; TP | 3.7E+10 | 10 | 0.1 | 5 | i.p. |
| 035551 | Tolerability | Control | 0.9% saline | | N/A | <1 | 0.1 | 5 | i.p. |
| 035730 | Tolerability | crT4 | 1X TBS, pH 7.4 + C/M | CF; TP | 6.0E+09 | 9.6 | 0.1 | 1 | i.p. |
| 035730 | Tolerability | Control | 1X TBS, pH 7.4 + C/M | | N/A | <1 | 0.1 | 1 | i.p. |

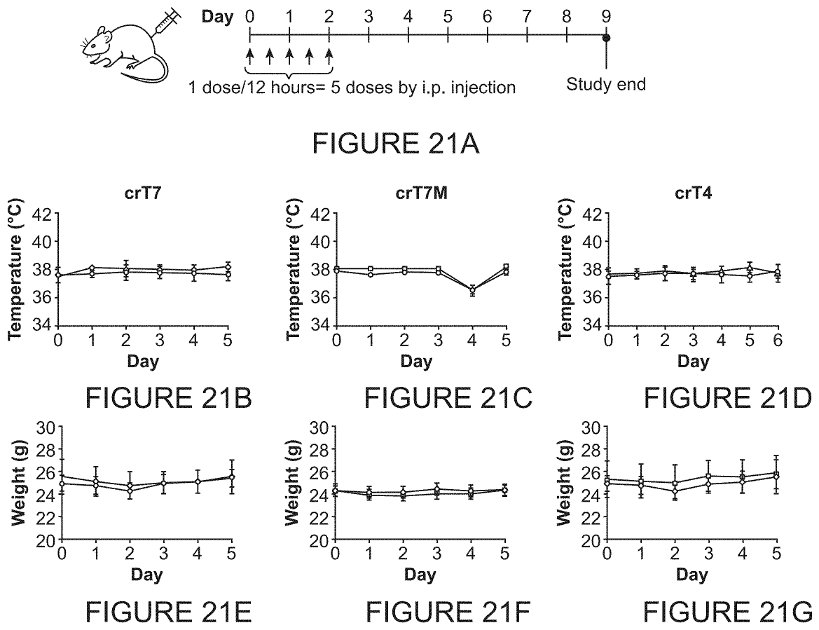

The treatment outline for tolerability is schematically shown in FIG. 21A. In the tolerability study, female CD-1 mice each received one 100-microliter dose per day by intraperitoneal injection for 5 days with 2.0×10¹¹ PFU/day/mouse of crT7, 5 days with 3.7×10⁹ PFU/day/mouse of crT7M or 1 day with 6.0×10⁸ PFU/day/mouse of crT4. crT7 and crT7m were suspended in sterile, endotoxin-free 0.9% saline, while crT4 was suspended in sterile, endotoxin-free 1× tris-buffered saline (pH 7.4) supplemented with 10 mM of each CaCl₂ and MgCl₂. No overt toxicity was observed during veterinary observation and no measurable changes in body temperature or body weight were noted after dosing with each crPhage preparation as shown in FIG. 21B-FIG. 21G.

### Example 18: LeuO Enhanced crPhages In Vivo Peritonitis Model Study

crPhages were prepared for the peritonitis model as described in Table 14 below:

TABLE 14
crPhages for an in vivo peritonitis model studies.

| | | Batch information | | | | | Dosing information | | |
|---|---|---|---|---|---|---|---|---|---|
| Study information | | | | Production | | | Dose | # | |
| Study | Study type | Phage | Diluent | notes | PFU/mL | EU/mL | (mL) | doses | RoA |
| 035552 | Peritonitis | crT7m | 0.9% saline | CF; TP | 3.7E+10 | 10 | 0.1 | 5 | i.p. |
| 035552 | Peritonitis | Control | 0.9% saline | | N/A | <1 | 0.1 | 5 | i.p. |
| 035468 | Peritonitis | crT7 | 0.9% saline | CF; TP | 2.0E+12 | 3 | 0.1 | 5 | i.p. |
| 035468 | Peritonitis | Control | 0.9% saline | | N/A | <1 | 0.1 | 5 | i.p. |
| 035730 | Peritonitis | crT4 | 1X TBS, pH 7.4 + C/M | CF; TP | 6.0E+09 | 9.6 | 0.1 | 1 | i.p. |
| 035730 | Peritonitis | Control | 1X TBS, pH 7.4 + C/M | | N/A | <1 | 0.1 | 1 | i.p. |

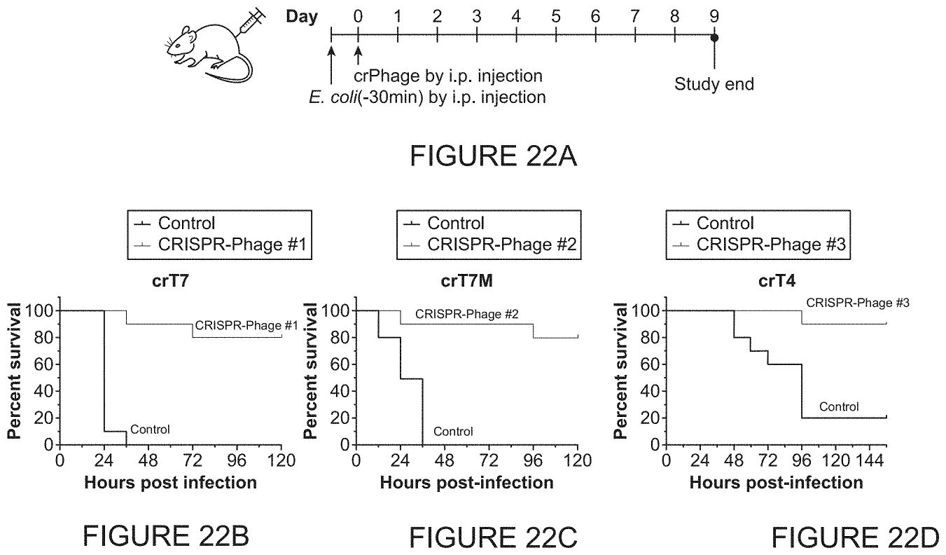

Based on the activity of three crPhages against a fecal E. coli isolate (ATCC 8739, crT7M and crT7) or lab (MG1655, crT4), the three crPhages were evaluated in a murine peritonitis model with E. coli. The treatment outline for the peritonitis model is schematically shown in FIG. 22A. Female CD-1 mice were injected intraperitoneally with a lethal dose of E. coli (˜5×10⁷ CFU/mouse of ATCC 8739) followed within 30 minutes by intraperitoneal injections of saline or crPhages. As before, crT7 and crT7m were suspended in sterile, endotoxin-free 0.9% saline, while crT4 was suspended in sterile, endotoxin-free 1×tris-buffered saline (pH 7.4) supplemented with 10 mM each CaCl₂ and MgCl₂. Single-dose administration of crPhage (2.0×10¹¹ PFU/dose of crT7, 3.7×10⁹ PFU/dose of crT7M or 6.0×10⁸ PFU/dose of crT4) resulted in significant protection in this acute, highly lethal bacterial challenge as seen in FIG. 22B-FIG. 22D. Control animals were treated with saline injections only. These data demonstrate that bacteriophages are able to infect and kill sufficient numbers of target bacteria to rescue lethal disease challenge in a relevant animal model of infection.

### Example 19: LeuO Enhanced crPhages In Vivo Bioburden Reduction in a Thigh Model

crPhages were prepared for an in vivo bioburden reduction in a thigh model as described in Table 15 below:

TABLE 15
crPhages for in vivo bioburden reduction in a thigh model studies.

| | | Batch information | | | | | Dosing information | | |
|---|---|---|---|---|---|---|---|---|---|
| Study information | | | | Production | | | Dose | # | |
| Study | Study type | Phage | Diluent | notes | PFU/mL | EU/mL | (mL) | doses | RoA |
| 035790 | Thigh | crT7m | 1X TBS, pH 7.4 | PE; CC | 2.0E+12 | <100 | 0.1 | 1 | i.m. |
| | Infection | | | | | | | | |
| 035790 | Thigh | crT4 | 1X TBS, pH 7.4 | CF; CC | 2.0E+11 | <1000 | 0.1 | 1 | i.m. |
| | Infection | | | | | | | | |
| 035790 | Thigh | crT7 | 1X TBS, pH 7.4 | PE; CC | 4.0E+12 | <100 | 0.1 | 1 | i.m. |
| | Infection | | | | | | | | |
| 035790 | Thigh | Cocktail | 1X TBS, pH 7.4 | | 1.0E+11 | <1000 | 0.1 | 1 | i.m. |
| | Infection | (crT7m/T7/T4) | | | each | | | | |
| 035790 | Thigh | Control | 1X TBS, pH 7.4 | | N/A | <1 | 0.1 | 1 | i.m. |
| | Infection | | | | | | | | |

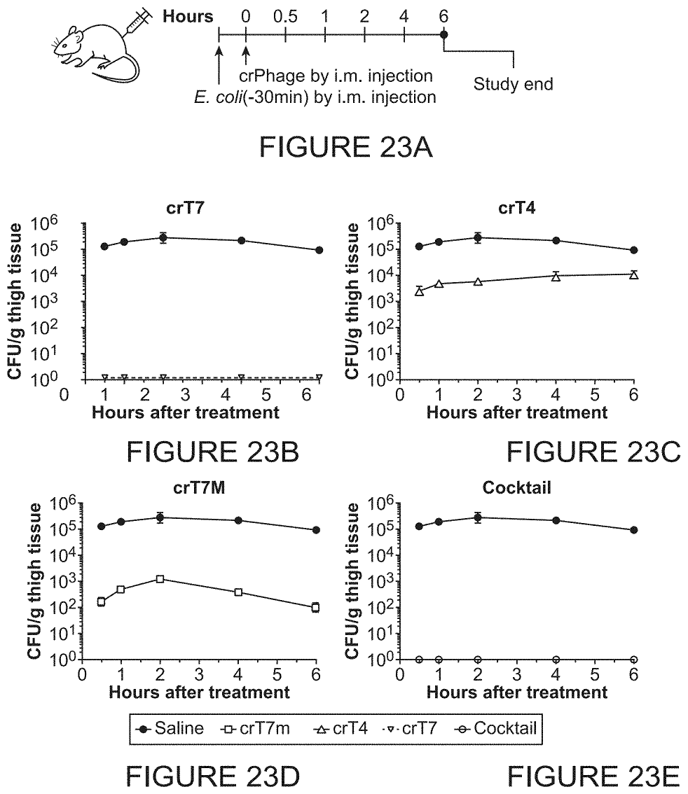

As a second demonstration of in vivo efficacy, a thigh infection model in mice was conducted to measure bioburden reduction after crPhage treatment. The treatment outline for this thigh model is schematically shown in FIG. 23A. The original crPhage stocks used in this study have a potency of 4.0×10¹² PFU/mL of crT7, 2.0×10¹² PFU/mL of crT7M, and 2.0×10¹¹ PFU/mL of crT4 with each phage suspended in sterile, endotoxin-free 1× tris-buffered saline (pH 7.4). The 3 crPhages were pooled into a cocktail with a final concentration of 1×10¹¹ PFU/mL of each phage containing an estimated endotoxin content of <10³ EU/mL.

One and four days prior to bacterial inoculation, female CD-1 mice were made neutropenic by intraperitoneal injection of 150 mg/kg cyclophosphamide into the left abdomen. Mice were inoculated with 10⁵ CFU of E. coli MG1655 by intramuscular injection into the thigh 30 minutes prior to intramuscular injection with the indicated crPhage or 1× tris-buffered saline (phage vehicle). Each individual crPhage or cocktail of 3 crPhages were administered by intramuscular injection into the same thigh with 100 microliters of crPhage solution, corresponding to a dose of 4.0×10¹¹ PFU/dose of crT7, 2.0×10¹¹ PFU/dose of crT7M, 2.0×10¹⁰ PFU/dose of crT4 or the cocktail containing 1.0×10¹⁰ PFU/dose of each phage. After injection with each crPhage, whole thigh muscles were excised at the indicated time points, homogenized and immediately diluted and plated to count surviving bacterial colonies per gram of tissue. CFU reductions measured approximately 2-log for crT4, 3-log for crT7M, and >5-log for both crT7 and the combined crPhage cocktail as seen in FIG. 23B-FIG. 23E. Taken together with the peritonitis results presented in FIG. 22B-FIG. 22D, these data demonstrate that crPhages have the potential to be highly effective antimicrobial agents in vivo.

### Example 20: LeuO Enhanced crPhages In Vivo Persistence and Distribution Studies Measured by Phage Titration

Given the unique PK/PD/ADME considerations of active phage-based products, the persistence and distribution of each crPhage in both target tissues and distal organs was evaluated to understand the kinetics of exposure in healthy animals. crPhages were prepared for an in vivo persistence and distribution study by intraurethral administration as described in Table 16 below:

TABLE 16
crPhages for in vivo persistence and distribution studies.

| | | Batch information | | | | | Dosing information | | |
|---|---|---|---|---|---|---|---|---|---|
| Study information | | | | Production | | | Dose | # | |
| Study | Study type | Phage | Diluent | notes | PFU/mL | EU/mL | (mL) | doses | RoA |
| 036239 | Persistence & | Cocktail | 1X TBS, pH 7.4 | CF | 2.0E+10 | Not | 0.05 | 1 | i.u. |
| | Distribution | (crT7m/T7) | | | each | measured | | | |
| 036239 | Persistence & | Control | 1X TBS, pH 7.4 | | N/A | <1 | 0.05 | 1 | i.u. |
| | Distribution | | | | | | | | |

Female CD-1 mice were treated with approximately 1.0×10⁹ PFU/dose/phage of a crT7/crT7m cocktail suspended in 1×tris-buffered saline (pH 7.4) by intraurethral instillation to N=3 mice per condition/time point. Intraurethral instillation was done by placement of a silicone-tipped syringe into the urethra of female mice and solution was injected directly into the bladder. Each mouse was dosed with an approximately 50 μL of vehicle or phage cocktail by intraurethral instillation while under isoflurane anesthesia. At time points of 0 (immediately following intraurethral administration), 0.5, 1, 6, 12, 24 and 72 hours post-inoculation, 3 mice per time point were sacrificed and collected bladder, kidney, blood, liver and spleen whole tissue homogenates were diluted and subjected to phage titration analysis to quantify the total combined amount of crT7 and crT7m. Means±standard error of the mean (SEM) shown are the result of 3 technical replicates from 3 animals and quantify plaque forming units per gram (bladder, kidney, liver, spleen) or per milliliter (blood) of either crT7 or crT7m (assay is not specific to either crPhage). crPhages were quantified as a pooled measurement using conventional phage titration against a known host that is susceptible to both crT7 and crT7m.

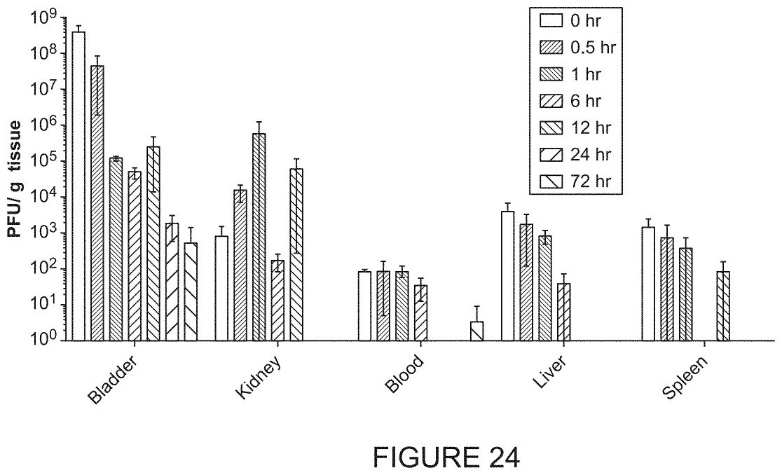

The presence of active crPhage was detected up to 72 hours after dosing as seen in FIG. 24. Apparent amplification during this time period was not observed which was expected due to the lack of a target E. coli replication host in the normal tissues and the inability to replicate in mammalian cells. Importantly, crPhage levels decreased over time in bladder and were undetectable in kidney, liver, blood and spleen by 72 hours, suggesting the absence of suitable E. coli replication hosts in treated animals results in loss of crPhage over time. Notably, significant phage titers were observed in the kidneys suggesting that intraurethral route of administration, in some cases, results in exposure in the lower and upper urinary tract. Also, significant phage titers were detected in blood, liver and spleen tissues, showing that crPhages appear to enter circulation by crossing the urothelium.

### Example 21: LeuO Enhanced crPhages In Vivo Persistence and Distribution Studies Measured by Quantitative PCR

A quantitative PCR-based method was developed and validated for the detection of each crPhage within a given cocktail (data not shown) enabling detection levels down to 50 copies per ng of total DNA in complex samples (e.g. commingled mouse blood and whole DNA). Quantitative PCR is a highly specific method to detect and quantify DNA and is theoretically able to measure the total amount of each engineered crPhage within samples as the primers are designed to recognize a specific phage genome containing an identical crRNA cassette insert. crPhages were prepared for an in vivo persistence and distribution study by oral administration as described in Table 17 below:

TABLE 17
crPhages for in vivo persistence and distribution studies.

| | | Batch information | | | | | Dosing information | | |
|---|---|---|---|---|---|---|---|---|---|
| Study information | | | | Production | | | Dose | # | |
| Study | Study type | Phage | Diluent | notes | PFU/mL | EU/mL | (mL) | doses | RoA |
| 035789 | Persistence & | Cocktail | 1X TBS, pH 7.4 | CF | 1.35E+10 | Not | 0.2 | 1 | oral |
| | Distribution | (crT7m/T7/T4) | | | each | measured | | | |
| 035789 | Persistence & | Control | 1X TBS, pH 7.4 | | N/A | <1 | 0.2 | 1 | oral |
| | Distribution | | | | | | | | |

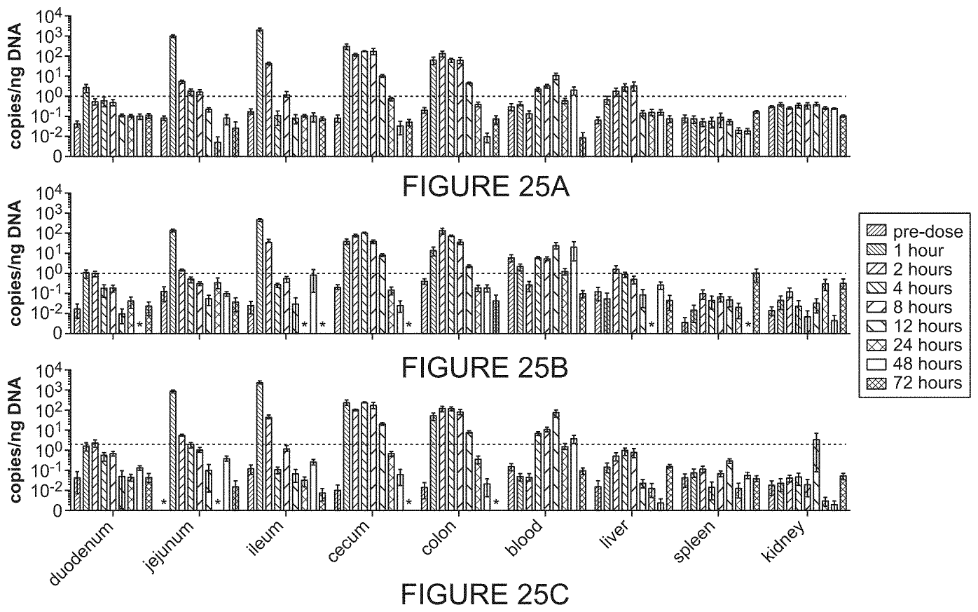

A small study was conducted in mice to determine the presence of multiple crPhages over time after a single oral administration of a crPhage solution. To mitigate potential phage degradation, mice were gavaged with 0.2 mL of 6% sodium bicarbonate to reduce stomach acid levels approximately 30 minutes prior to crPhage dosing. A single dose of 2.7×10⁹ PFU total of each crT7, crT7m and crT4 in 200 μL 1× tris-buffered saline (pH 7.4) was administered by oral gavage to N=3 mice per condition/time point. Animals were sacrificed at various time points and total DNA from whole tissue homogenates was extracted and subjected to qPCR analysis to quantify the amount of crT7 (FIG. 25A), crT4 (FIG. 25B) or crT7m (FIG. 25C) present. Means±standard error of the mean (SEM) shown are the result of 3 technical replicates from 3 animals.

As shown in FIG. 25A-FIG. 25C, it was possible to successfully detect each crPhage during transit through the GI tract across and also in feces. By 72 hours, significant quantities of the crPhage cocktail were not detectable in any tissue, demonstrating that the crPhage is successfully cleared. These data corroborate the observation of loss of crPhages over time after a single intraurethral administration as quantified by phage titration in FIG. 24. Notably, these data suggest that oral administration of crPhages results in systemic exposure, specifically in blood and liver tissues, as observed following intraurethral administration in FIG. 24.

### Example 22: LeuO Enhanced crPhages Non-GLP Toxicology Study

A test article of crPhage cocktail containing crT7 and crT7m was administered either intravenously (1.0×10¹¹ PFU/dose/phage) or by intracatheter instillation into the bladder (0.5×10¹¹ PFU/dose/phage) once daily for 7 consecutive days to female Cr1:CD-1 mice. crPhages were prepared for an the 7-day toxicology study as described in Table 18 below:

TABLE 18
crPhages for 7-day toxicology study.

| | | Batch information | | | | | Dosing information | | |
|---|---|---|---|---|---|---|---|---|---|
| Study information | | | | Production | | | Dose | # | |
| Study | Study type | Phage | Diluent | notes | PFU/mL | EU/mL | (mL) | doses | RoA |
| 035938 | 7-day | Cocktail | 1X TBS, pH 7.4 | PE; CC | 1.0E+12 each | Not | 0.1 | 7 | i.v. |
| | Toxicology | (crT7m/T7) | | | | measured | | | |
| 035938 | 7-day | Control | 1X TBS, pH 7.4 | | N/A | <1 | 0.1 | 7 | i.v. |
| | Toxicology | | | | | | | | |
| 035938 | 7-day | Cocktail | 1X TBS, pH 7.4 | PE; CC | 1.0E+12 each | Not | 0.05 | 7 | i.u. |
| | Toxicology | (crT7m/T7) | | | | measured | | | |
| 035938 | 7-day | Control | 1X TBS, pH 7.4 | | N/A | <1 | 0.05 | 7 | i.u. |
| | Toxicology | | | | | | | | |

The crPhage preparation was prepared using sterile, endotoxin-free 1× tris-buffered saline (pH 7.4). Groups 1 and 2 (9 female mice/group) were dosed with 0.1 mL of vehicle (1× tris-buffered saline, pH 7.4) or test article in a tail vein. Groups 3 and 4 (9 female mice/group) were dosed with 0.05 mL of vehicle or test article into the bladder using a catheter syringe. Group assignment and dosage levels are described in Table 19 below:

TABLE 19
Group assignment and dose levels for non-GLP toxicology study.

| | | | | | # of |
|---|---|---|---|---|---|
| | | | | | Animals for |
| Dose | # of | Test | | Dose Volume | Necropsy |
| Group | Animals | Article | Dose Route | (mL/animal) | Day 8 |
| 1 | 9 | Vehicle | Intravenous | 0.1 | 6 |
| 2 | 9 | crPhage | Intravenous | 0.1 | 6 |
| 3 | 9 | Vehicle | Intraurethral | 0.05 | 6 |
| 4 | 9 | crPhage | Intraurethral | 0.05 | 6 |

Animals were monitored for clinical signs twice daily over the duration of the study. Detailed clinical observations were performed once during the pre-dose period and prior to necropsy on Day 8. Body weights were measured once during the pre-dose period and on Days 1, 3, and 7. Food consumption was measured during the 7-day dosing period. On Day 8, six animals per group were randomly chosen for necropsy, and the remaining three were discarded without necropsy. At necropsy, body weights were collected from animals fasted for at least 4 hours and were used for calculation of organ weights relative to body weight. Clinical pathology assessments, hematology (3 mice/group) and serum chemistry parameters (3 mice/group), were performed on the day of the scheduled necropsy. Postmortem assessment included necropsy and measurement of selected organ weights. A full tissue list was collected at necropsy. Collected tissues were sectioned with one section frozen in liquid nitrogen (stored frozen at ≤−70° C.) and a second section was preserved in 10% neutral-buffered formalin.

There were no crPhage-related mortality or morbidity, no effect on body weight, and no abnormal clinical observations for either route of administration. Phage-related effects on hematology were limited to a lower hemoglobin level and decreases in other RBC mass-related parameters, increased reticulocyte counts, and decreased eosinophil count in the crPhage IV-treated group. crPhage-related effects on serum chemistry were limited to higher cholesterol and triglyceride levels in the crPhage IU-treated group. crPhage-related effects on organ weights (absolute and relative to body and brain weights) consisted of increased spleen and kidney weights and decreased lung weights in the crPhage IV-treated group.

In conclusion, once daily IV or IU administration of crPhage was well tolerated. Possible test article-related effects in the Phage IV-treated group were limited to decreases in red blood cell mass-related parameters, increased reticulocyte counts, decreased eosinophils, and increased spleen, kidney, and decreased lung weights. Possible test article-related effects in the Phage IU-treated group were limited to higher cholesterol and triglyceride levels. The clinical chemistry and organ weight results from this study should be viewed in the context of the small sample sizes used (3 for clinical chemistry and 6 for organ weights) and the possibility that these alterations are artifacts due to small sample size.

### Example 23: Identifying a LeuO Equivalent in C. difficile

The CRISPR-Cas operon in C. difficile is not regulated by LeuO and H-NS as seen in E. coli. Rather, regulation is regulated by glucose in ccpA-dependent manner in the absence of ccpA binding site. Notably, the upregulation of CD2983 has been associated with the upregulation of the Cas operon during a nutrient shift. Further, the regulation of CD2983 appears to be controlled by CodY, a global stringent response regulator. The loss of proteins similar in sequence (>40% similarity) in a type I-D system has been shown to result in increased expression off the Cas operon.

As such, it is possible that CodY and CD2983 are analogous equivalents to HNS and LeuO in C. difficile. The similarity between both systems is summarized in Table 20 below:

TABLE 20
Summary of characteristics for Cas operon regulation
in E. coli and C. difficile.

| Gram Negatives (e.g. E. coli) | Gram Positives (e.g. C. difficile) |
|---|---|
| Cas regulated by nutrient | Cas regulated by nutrient |
| conditions | conditions |
| HNS global repressor | CodY global repressor |
| LeuO is regulated by ppGpp | CodY is regulated by GTP, BCAAs |
| LeuO impacts BCAA biosynthesis | CodY impacts BCAA biosynthesis |
| LeuO contains N-term HTH | CD2983 contains N-term HTH |
| LeuO global activator | CD2983 specific regulator? |

### Example 24: Engineering and Validation of a Lysogeny Module Knockout Bacteriophage

Engineering—A plasmid containing homology arms flanking the lysogeny region in ϕCD24-2 and a counterselective crRNA targeting the lysogeny region was designed in silico and synthesized by BioBasic. The plasmid was transformed into E. coli and conjugated into C. difficile strain CD19. ϕCD24-2 was amplified on CD19 carrying the engineering plasmid. The resulting phage population was PCR screened for the presence of engineered phages. If the bulk PCR screen was positive, the lysate was plagued and individual plaques were screened for the cI repressor gene knockout. Pure engineered phage was amplified to high titer for use in validation studies.

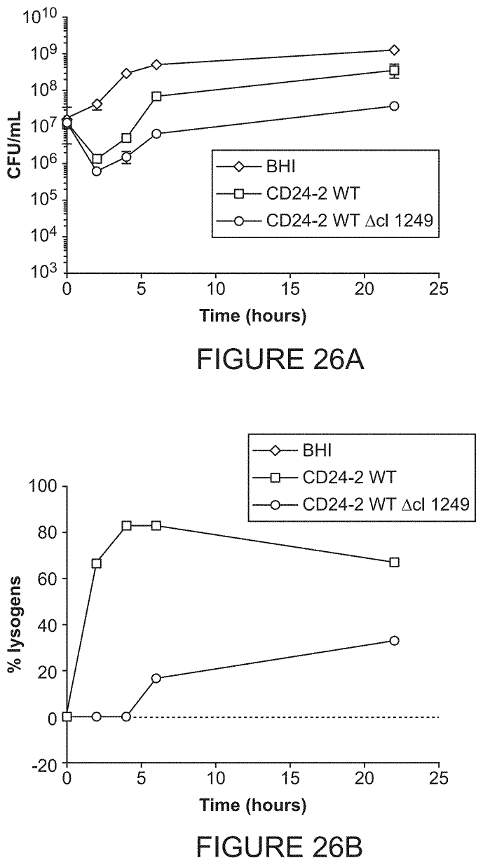

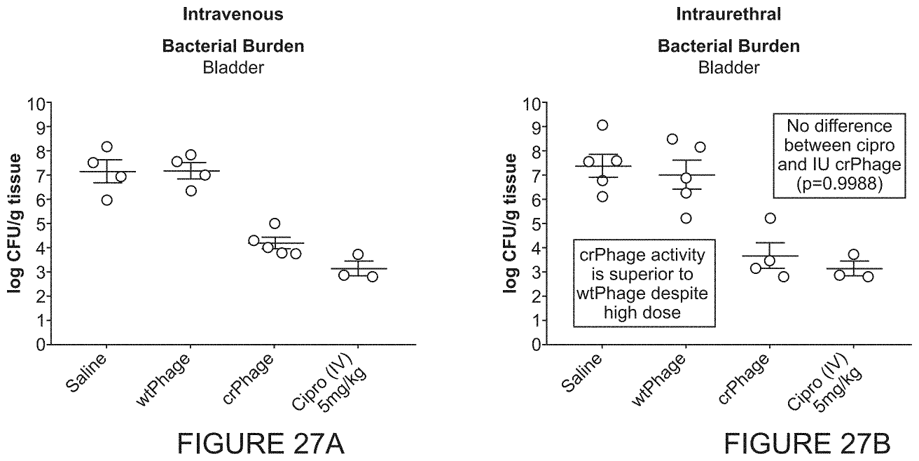

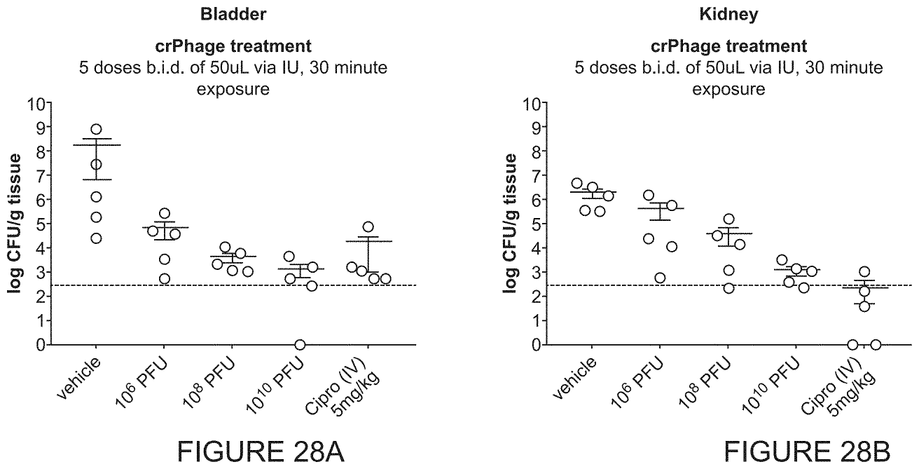

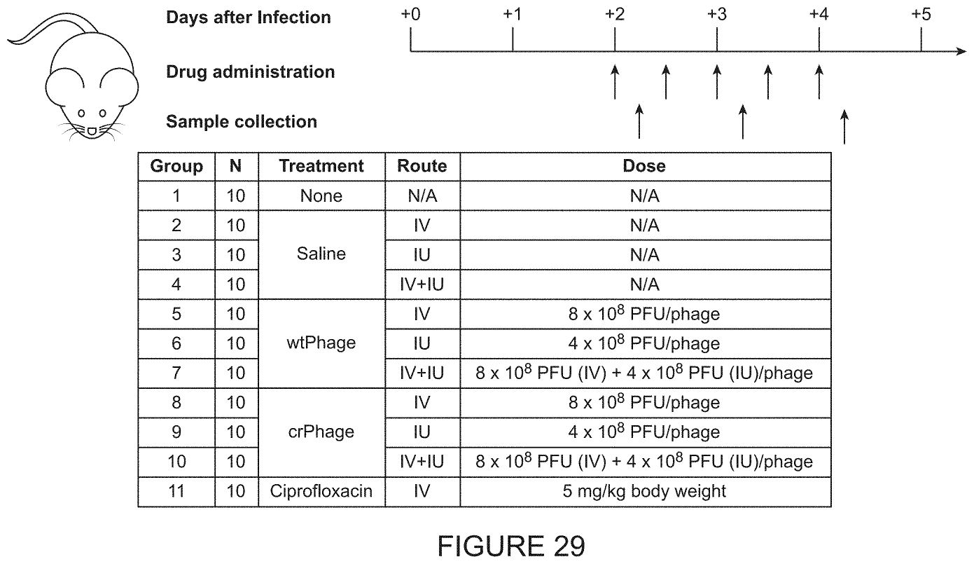

Validation—CD19 culture at mid-log phase was treated with BHI (growth medium, no treatment control), with WT CD24-2 or with ΔcI CD24-2 (i.e. a lysogeny module knockout). FIG. 26A exemplifies the number of surviving cells (CFU/mL) counted at various time points after the treatment. Surviving cells were further screened for the presence of lysogenized CD24-2. FIG. 26B exemplifies the % lysogens present at various time points after the treatment.

### Example 25: Treatment of a Microbiome-Related Disorder

To tune a subject's microbiome, a pharmaceutical composition comprising an engineered bacteriophage as described herein can be administered to the subject. The pharmaceutical composition can modulate or kill singular or plural bacterial populations within the microbiome by CRISPR-Cas activity, lytic activity, or a combination thereof.

### Example 26: UTI Efficacy Study Illustrating Reduction in E. coli in Bladder and Kidney Following Intraurethral (IU) or Intravenous (IV) Administration

Mice colonized with NC101 were treated with saline, a phage cocktail (2.4×10¹⁰ PFU/mL); or ciprofloxacin. Ciprofloxacin (1-cyclopropyl-6-fluoro-1,4dihydro-4-oxo-7-(1)-3-quinolinecarboxylic acid hydrochloride) is an antibiotic, while effective, use results in a myriad of side effect and bacterial resistance.

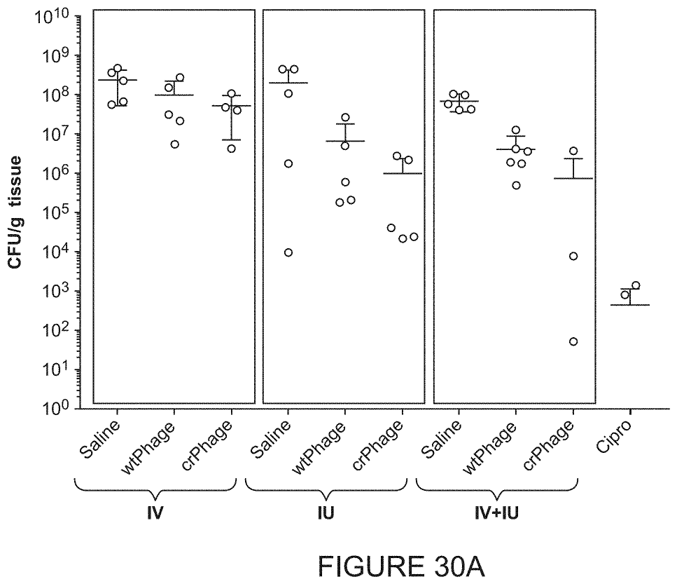

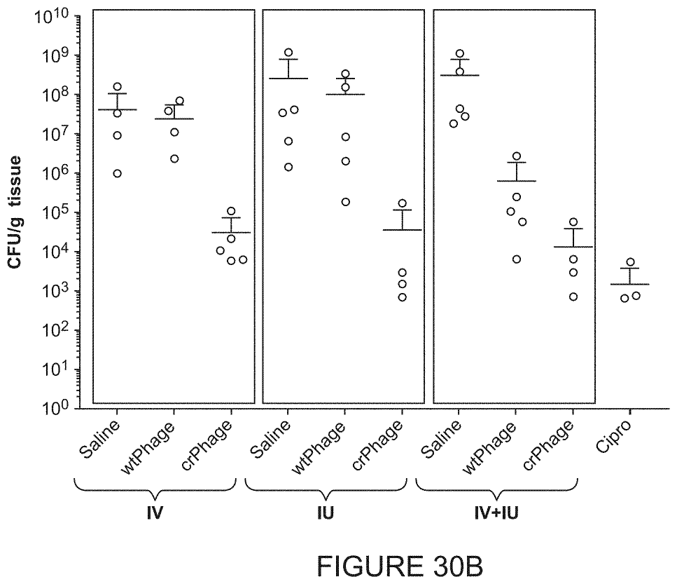

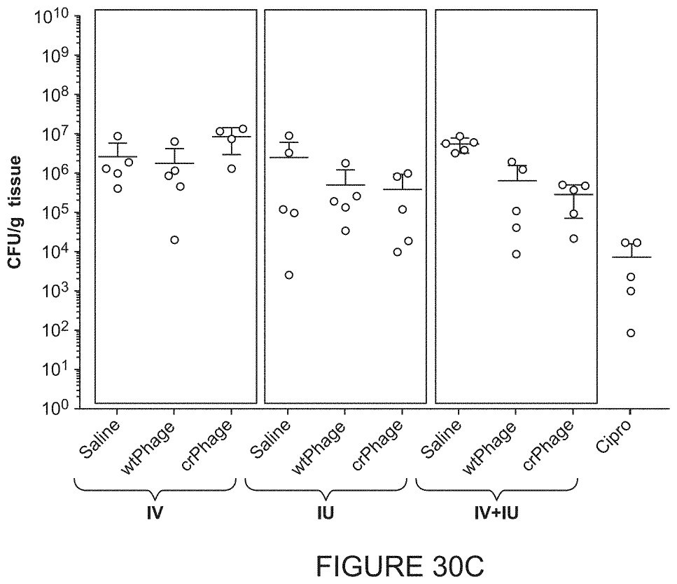

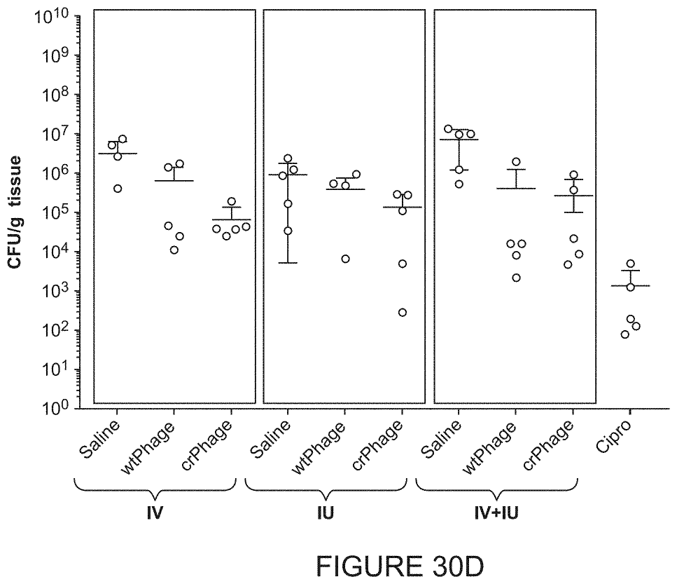

In this example, tissues were collected after a single dose or after 5 doses and analyzed for CFUs. Phage treatment reduces CFUs in both the bladder (FIG. 30A-FIG. 30B) and kidneys (FIG. 30C-FIG. 30D). The results exemplify that IU and IV delivery of phage reduces CFUs in the bladder. Further, the results exemplify that crPhage cocktail has 1.5- to 3.5-log improved kill over wtPhage cocktail at 120 h in the bladder (FIG. 30B). The results also exemplify that regardless of delivery route, at 120 h crPhage cocktail performs comparable with ciprofloxacin in the bladder.

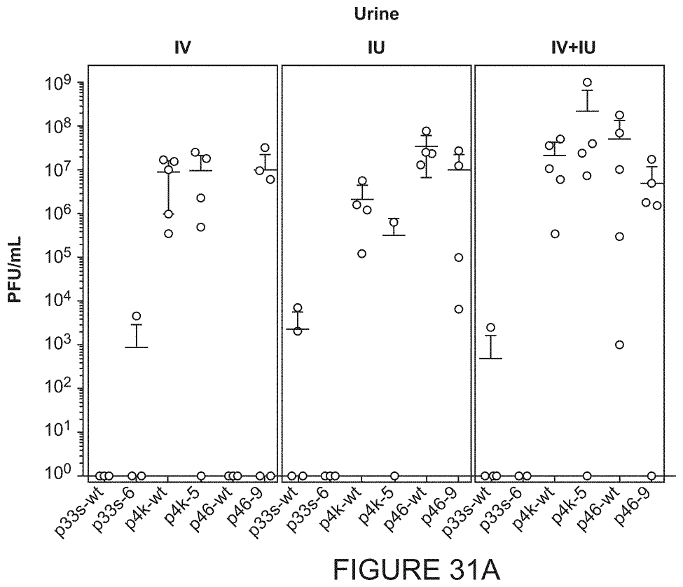

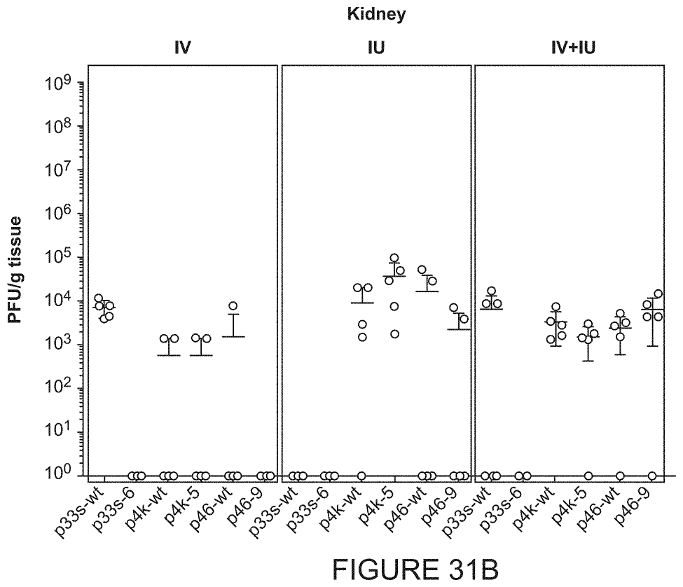

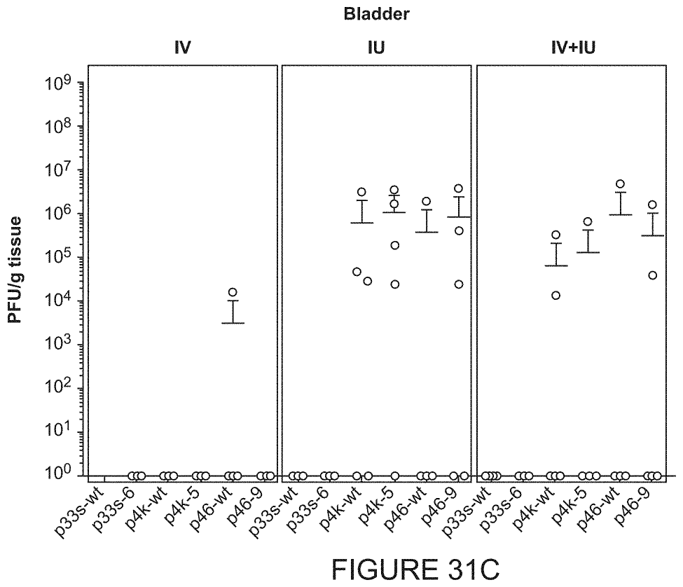

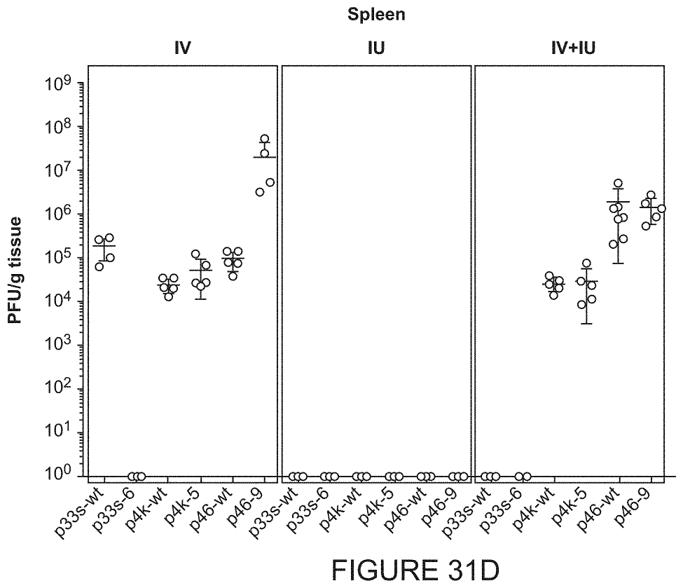

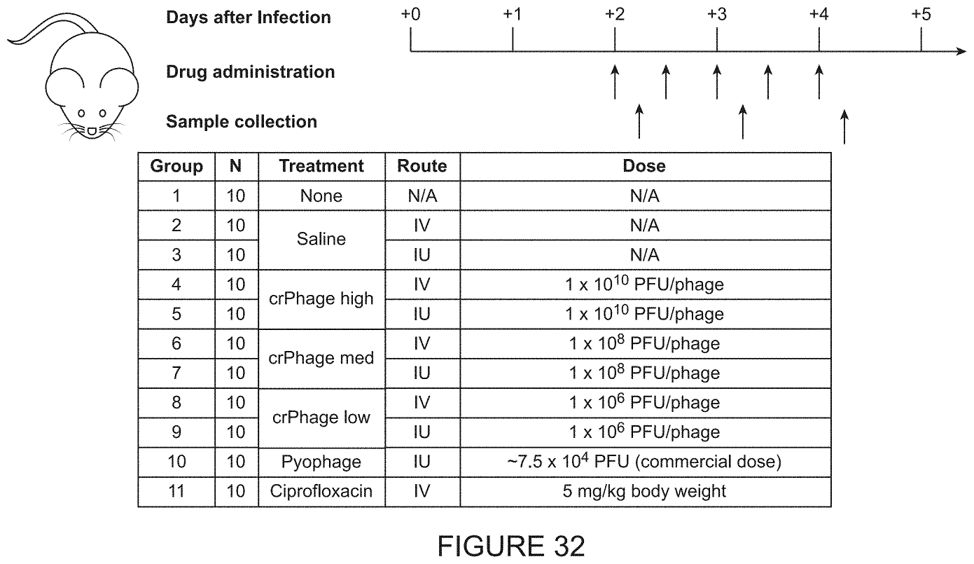

The study further illustrates route-dependent penetration of phage into different tissues and fluids, such as urine (FIG. 31A), kidney (FIG. 31B), bladder (FIG. 31C), and spleen (FIG. 31D). The results illustrate that there is measurable phage in the urine regardless of treatment route.

### Example 27: UTI Efficacy Study Comparison of Research-Grade Material Compared WT Versus Engineered Cocktail Via Different Administration Routes

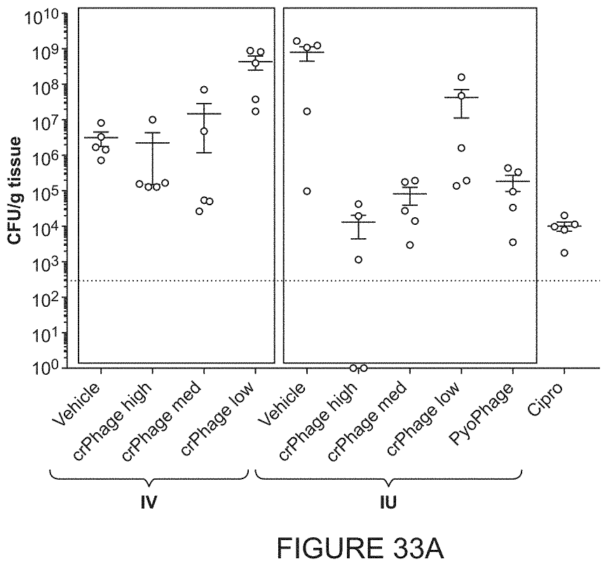

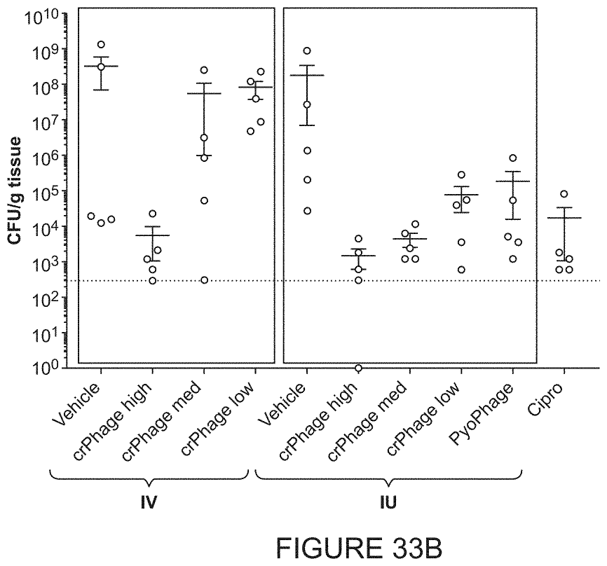

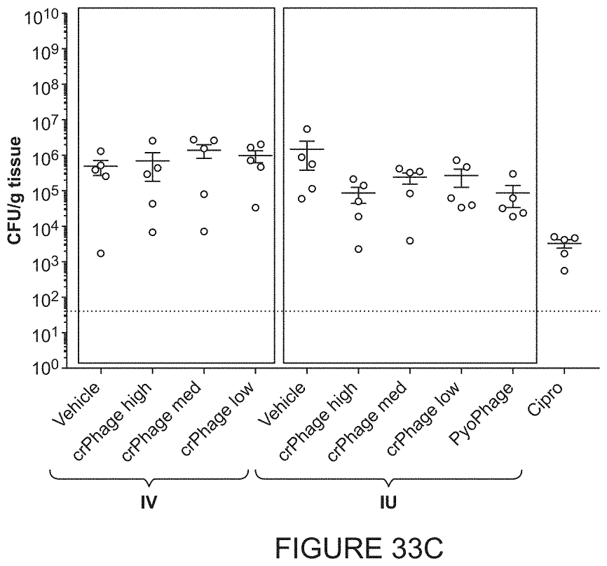

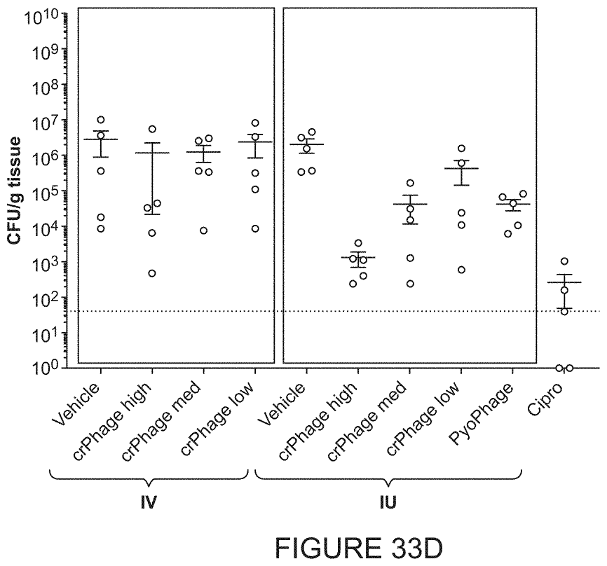

Mice colonized with NC101 were treated with saline; a phage cocktail (2.4×10¹⁰ PFU/mL); or ciprofloxacin. Tissues were collected after a single dose or after 5 doses and analyzed for CFUs. Phage treatment reduces CFUs in both the bladder (FIG. 33A-FIG. 33B) and kidneys (FIG. 33C-FIG. 33D). The results exemplify that IU and IV delivery of phage reduces CFUs in the bladder. Further, the results exemplify that high titer crPhage cocktail has 5.1-log improved kill over vehicle at 120 hour in the bladder (FIG. 33B), and that regardless of the delivery route, a high dose crPhage outperforms ciprofloxacin in the bladder at 120 hours (FIG. 33B).

### Example 28: Clinical Trial to Assess the Safety, Pharmacokinetics and Potential Efficacy of crPhage in E. coli Colonized Adults

Phase 1b—Safety, tolerability and PK study is conducted in patients with urinary tract colonization by maximum feasible dose BID via catheter instillation. Pharmacokinetics related to phage persistence time, distribution and elimination is confirmed in the bladder. Bacteriophages that are non-toxic and unable to infect human cells give high therapeutic index.

Phase 2—Non-inferiority, 2:1 randomized versus standard of care, double-blind clinical trial is conducted in adult patients with recurrent urinary tract infections (UTI) including MDR and CR strains and in patients with pyelonephritis. IV administration and dosing regimen is evaluated.

Primary endpoint: resolution of UTI symptoms at end of therapy and test of cure (TOC—7 days after end of therapy) with demonstration of bacterial pathogen reductions≤10³ CFU/mL on urine culture (Microbiological Success) measured at TOC.

Secondary endpoint: Immunogenicity (presence of anti-phage antibodies), QoL measures (pain/discomfort, etc), durability of response.

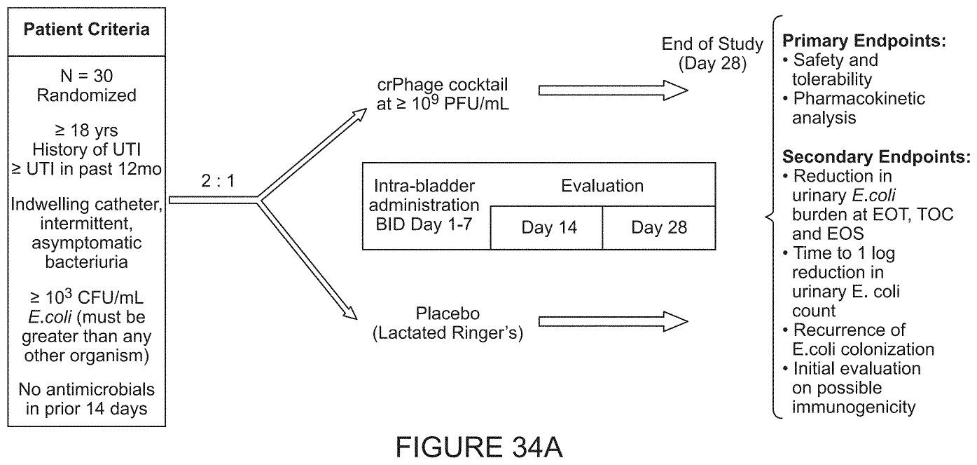

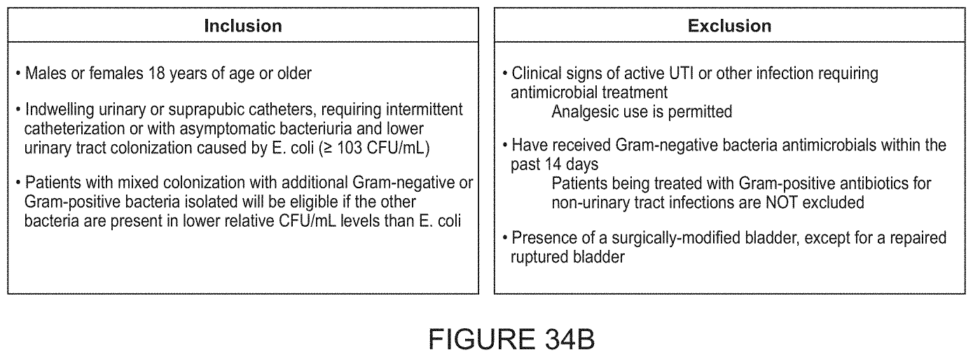

FIG. 34A is a schematic of an exemplary human study conducted in adults with reoccurring and asymptomatic E. coli colonization of the urinary tract. FIG. 34B is an exemplary study participant inclusion and exclusion criteria for the UTI Phase 1b study.

### Example 29: Phase 1b Design in CDAD Patients

Phase 1: Safety, tolerability and PK study is conducted in healthy volunteers (C. diff colonization naturally occurring). 10-14 day oral dosing regimen and 28 day follow-up is evaluated. Safety and tolerability is evaluated. PK analysis of phage and C. difficile in stool is evaluated over time.

Phase 2: Non-inferiority study, 2:1 randomized, active vs. standard of care (i.e. vancomycin), double blind study in 2nd and 3rd line CDI patients
Exploratory dose finding
Active crPhage cocktail vs vancomycin orally for 10-14 days (or, if unresolved, until resolution of symptoms)
Primary endpoint: time to resolution of diarrhea (TTROD), recurrence of CDAD (time to diarrhea) and grade of diarrhea assessed up to 8 weeks following completion of treatment period, safety and tolerability assessment, PK analysis
Secondary endpoint: immunogenicity (presence of anti-phage antibodies), additional QoL measures, durability of response

Phase 3: Efficacy study in larger patient population of first line and recurrent CDI compared to SOC (vancomycin) orally for 10-14 day dosing regimen (40 day study), sub-analysis of front line vs. recurrent patient treatment
Placebo-controlled double-blinded study targeting initial FDA interactions will determine what expanded data sets are necessary
If Phase 2 data provides sufficient evidence of efficacy and safety smaller data sets may be sufficient

### Example 30: Engineered Phages Show Increased Killing Against Both Type IE and Type IF E. coli Strains

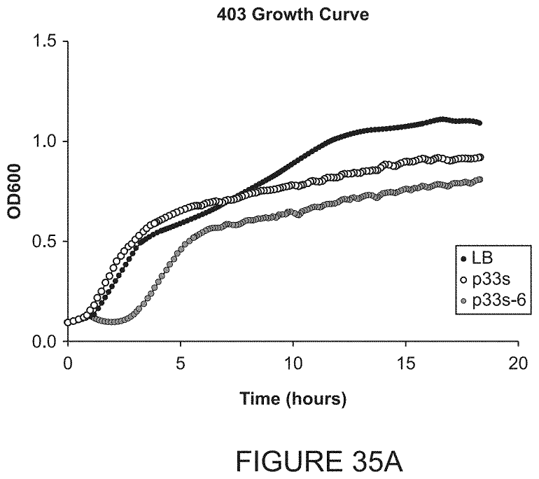

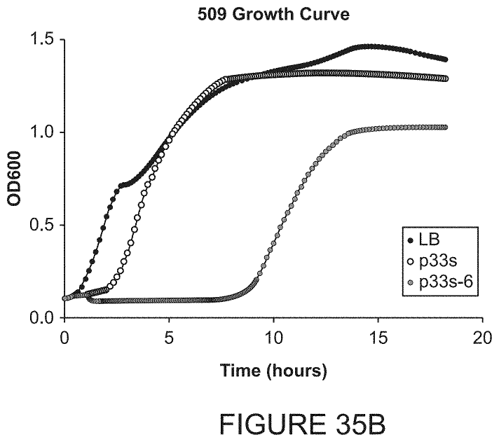

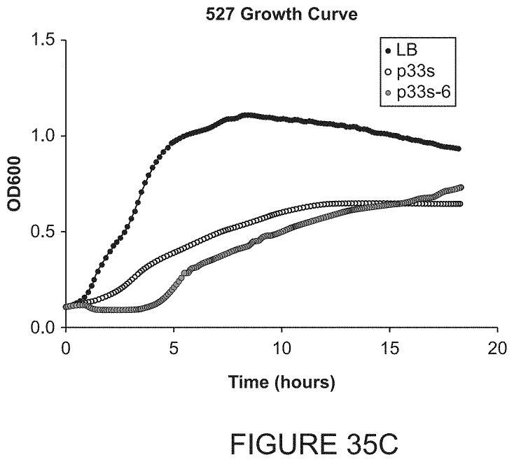

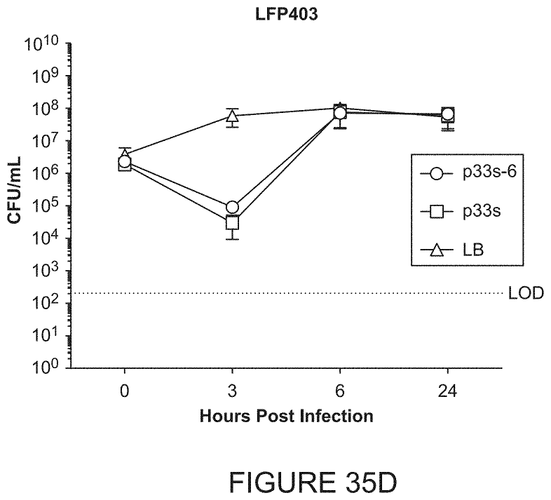

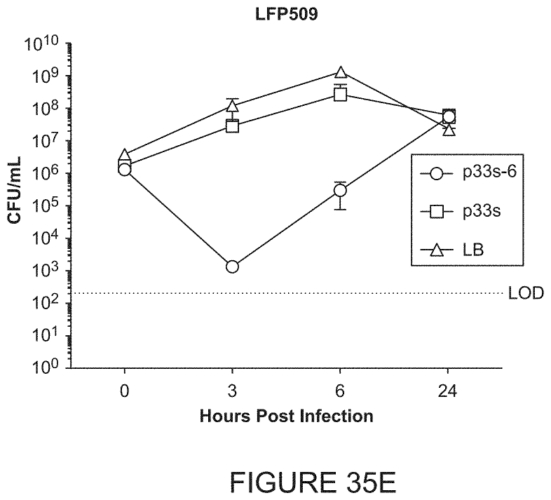

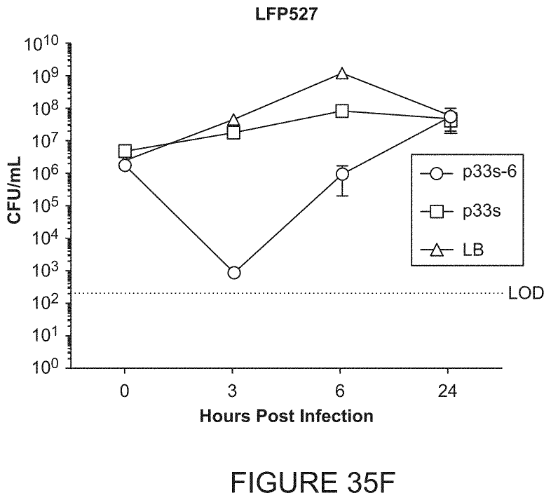

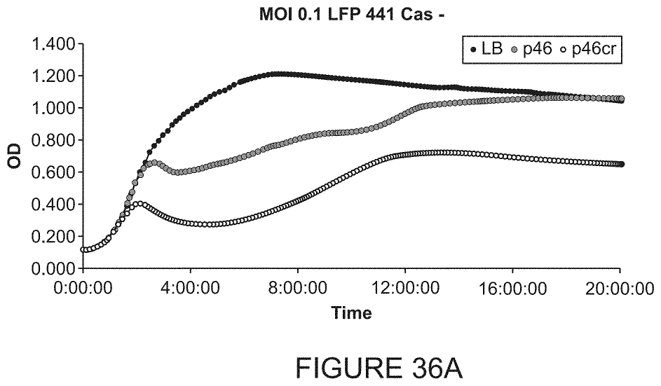

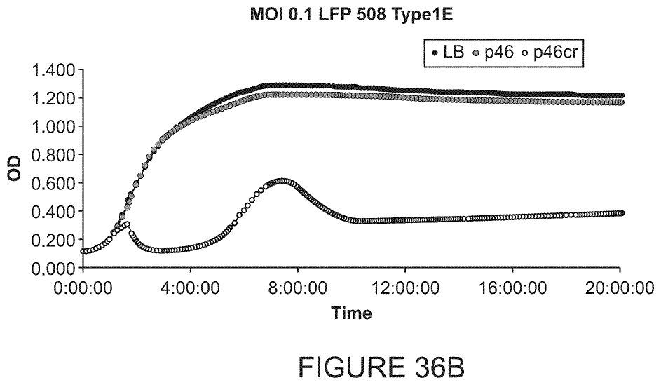

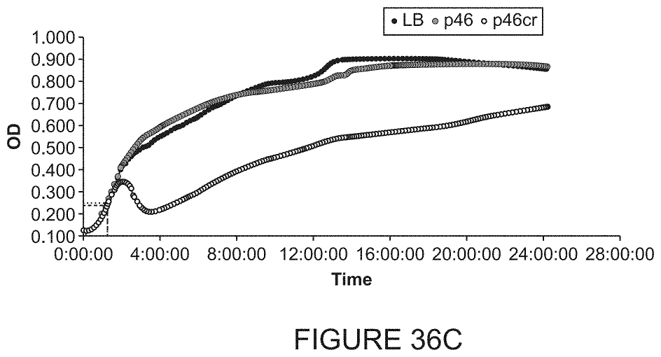

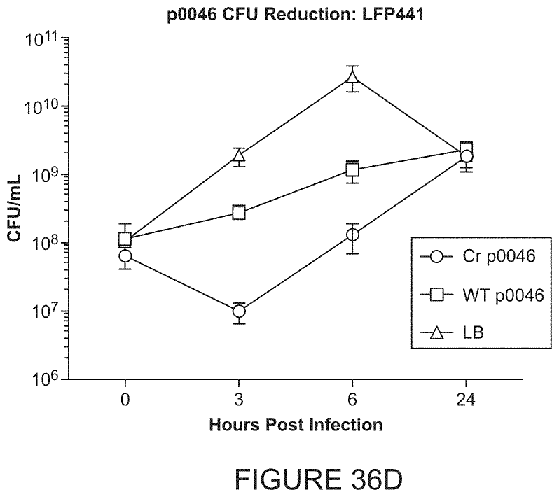

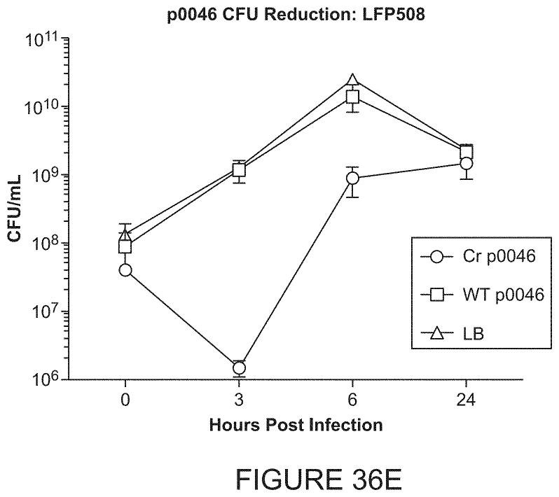

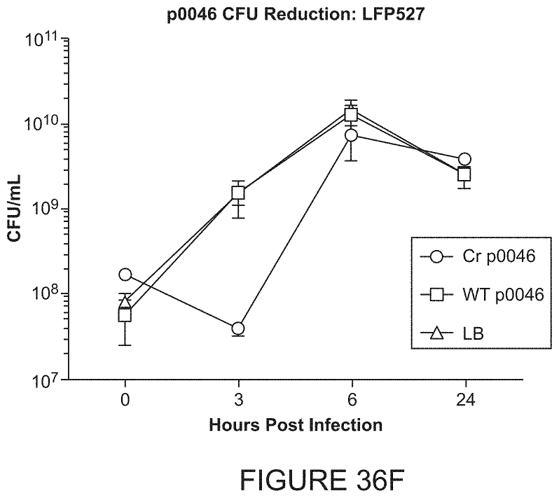

For all host range and CFU reduction experiments, the strains and phages were prepared as follows. All experiments were performed in 96 well flat bottom clear plates with total final volume of 200 uL in LB with salts (10 mM MgCl₂ and CaCl₂). Three strains were selected to compare WT and CRISPR phages. Each E. coli strain was placed in a microtiter plate either alone, with WTphage (p33s; p46), or with the crPhage (p33s-6; p46cr) at MOI 0.1. For host range experiments, the cultures were incubated at 37° C. for 20 hours in a plate reader to monitor growth of populations by optical density (OD; 600 nm). For CFU reduction experiments, the cultures were incubated at 37° C. for 24 hours in a shaking incubator and aliquots were taken at (0, 3, 6, and 24 hours). Results are exemplified in FIG. 35A-FIG. 35F, and FIG. 36A-FIG. 36F.

### Example 31: Switching Phage Cocktails Overcomes Target Bacterial Resistance in E. coli

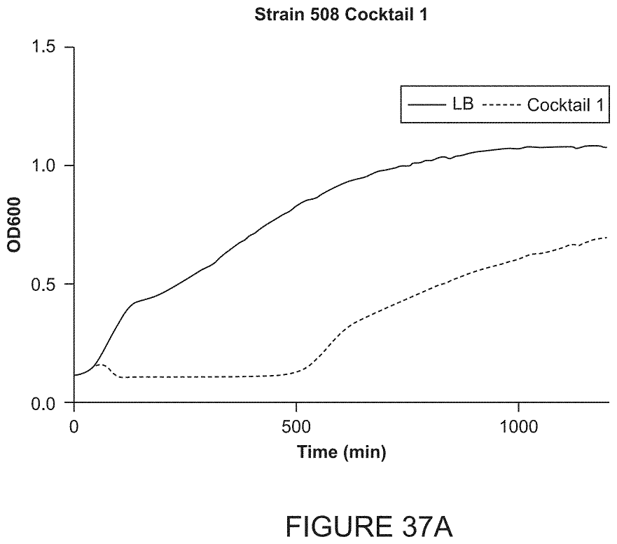

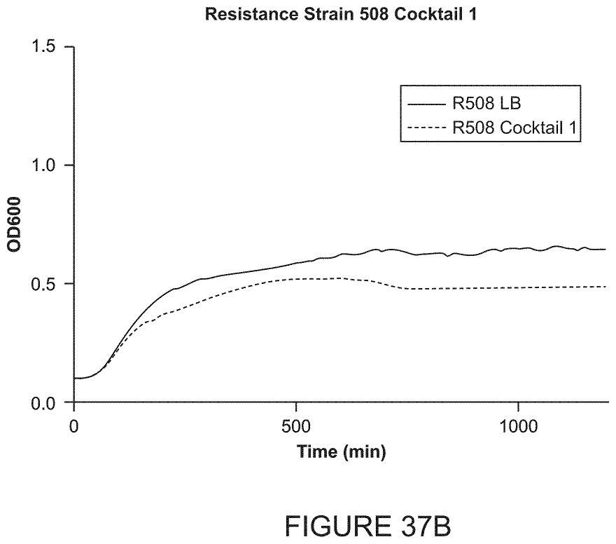

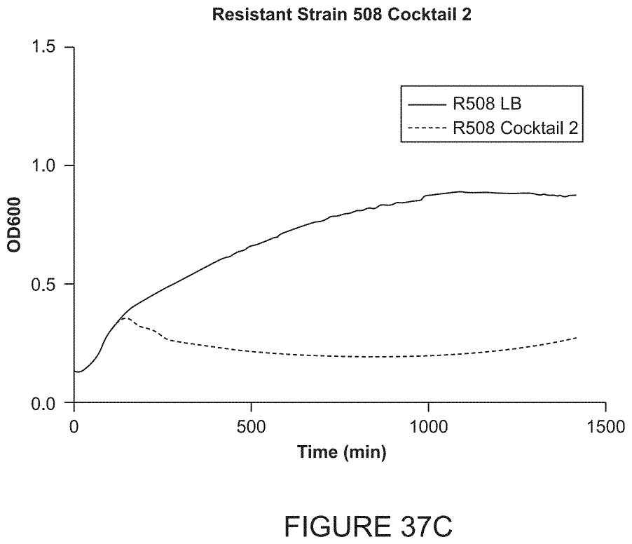

Host range experiments were setup as described above for crCocktail (cr33s, cr46, cr4k) against strains 508 and 527. After 24 hours only strain 508 yielded viable colonies. The colonies were re-streaked 3× to remove any phage. The 508 resistant strains were reevaluated with the crCocktail and WT cocktail 2 (pF0, pJ0, pJc, pE8, pE4, and pJ4) using the same host range protocol. Results are exemplifies in FIG. 37A-FIG. 37C.

### Example 32: Comparison of Wild Type Phage PB1 and CRISPR-Enhanced PB1 Against Pseudomonas aeruginosa Strains

A panel of 44 P. aeruginosa strains was mixed with either LB, PB1 or cr-PB1 at an MOI of 0.01 (˜5×10⁵ bacteria and 5×10³ phage or LB). Strains+phage or LB were grown for 20 hours at 37° C. and the optical density at 600 nm (OD₆₀₀) was measured every hour to generate a growth curve. A Riemann sum of the values from t=4 hrs to t=12 hrs were calculated for uninfected, PB1-infected and cr-PB1-infected cultures. Results are exemplified in Table 21. The values in the table represent the ratio of the area under the curve (AUC) for PB1 or cr-PB1 (PB.Engineered) compared to the uninfected control. A smaller number represents a larger decrease in optical density. Gray boxes indicate AUC ratios<0.7. Hit percentage is the percent of strains with an AUC ratio<0.7.

TABLE 21

| | PB1 | cr-PB1 |
|---|---|---|
| b1031 | 0.963297 | 0.870552 |
| b1045 | 1.717988 | 1.011726 |
| b1066 | 0.96017 | 0.98204 |
| b1046 | 0.971213 | 0.969247 |
| b1073 | 1.024263 | 1.017944 |
| b1048 | 0.976147 | 0.998046 |
| b1074 | 0.83458 | 1.072608 |
| b1050 | 0.999267 | 1.001961 |
| b1075 | 0.998144 | 1.021623 |
| b1051 | 0.954393 | 0.890835 |
| b1055 | 0.860689 | 1.192755 |
| b1076 | 0.966672 | 0.99161 |
| b1052 | 0.982928 | 0.947011 |
| b1079 | 0.350342 | 0.440958 |
| b1054 | 0.879394 | 0.987756 |
| b1084 | 1.025938 | 0.987852 |
| b1034 | 1.007372 | 1.028967 |
| b1058 | 0.953205 | 1.006118 |
| b1059 | 0.925737 | 0.983083 |
| b1035 | 0.229912 | 0.20969 |
| b1061 | 0.931473 | 0.993693 |
| b1041 | 0.970653 | 0.970961 |
| b1085 | 1.023776 | 1.001058 |
| b1126 | 0.99979 | 0.969054 |
| b1102 | 1.013489 | 0.998923 |
| b1127 | 0.136604 | 0.13795 |
| b1128 | 0.183435 | 0.164191 |
| b1109 | 0.948729 | 0.742165 |
| b1138 | 0.982688 | 0.967086 |
| b1110 | 0.261029 | 0.258299 |
| b1111 | 1.035309 | 0.969425 |
| b1118 | 1.084256 | 1.036543 |
| b1233 | 0.423587 | 0.253027 |
| b1112 | 1.05943 | 1.041742 |
| b1033 | 0.989803 | 1.002004 |
| b1117 | 1.023928 | 1.026984 |
| b1099 | 1.006169 | 1.150375 |
| b1086 | 1.019048 | 1.014256 |
| b1121 | 0.129781 | 0.126992 |
| b1090 | 1.068717 | 1.064457 |
| b1122 | 0.180161 | 0.181129 |
| b1092 | 0.54725 | 0.532722 |
| b1125 | 1.05666 | 0.986049 |
| b1100 | 0.763823 | 0.768105 |
| Hit Percentage | 18.75 | 18.75 |

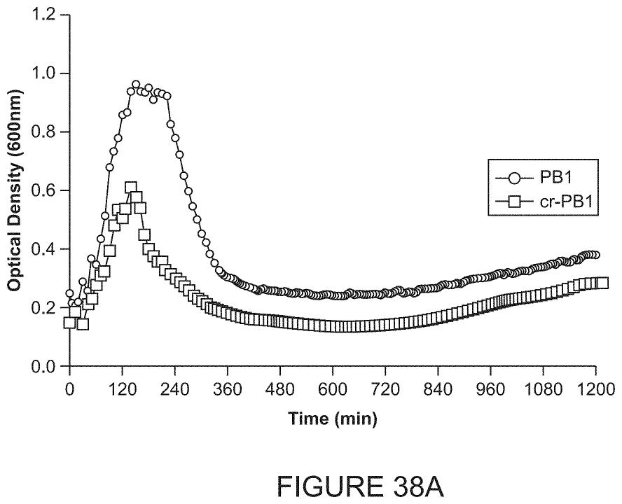

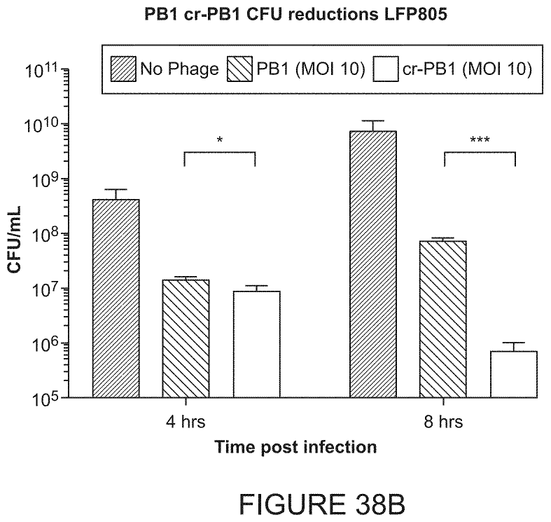

Growth curves: A stationary phase culture of LFP805 was diluted to a concentration of ˜10⁷ CFU/ml in supplemented LB (LB+10 mM MgCl₂ and 10 mM CaCl₂). Stocks of wild-type PB1 and cr-PB1 phage were diluted to a concentration of 10⁵ PFU/ml in supplemented LB. Bacterial and phage stocks were mixed 1:1 (MOI of 0.01) and grown in a plate reader with aeration at 37° C. for 20 hours. The OD₆₀₀ was measured every 10 min. Growth curves are exemplified for PB1 and cr-PB1 in FIG. 38A.

CFU Reductions: Stationary phase cultures of LFP805 were diluted to an optical density of 1.0 and 10 μL of diluted cultures were added to 180 μL of LB (˜10⁵ bacteria). PB1 and cr-PB1 were diluted to 10⁸ PFU/ml and either 10 μL of phage (10⁶ PFU) or 10 μL of LB were added. Cultures were grown with aeration at 37° C. and 10-fold dilutions were plated on LB agar at 4 hrs and 8 hours post-inoculation to determine the CFU/ml in each culture. Calculated CFU/ml at 4 hours and 8 hours are exemplified for uninfected (black bars), wild-type PB1 infected (light gray bars) or cr-PB1 phage infected cultures (medium gray bars) in FIG. 38B. CFU were compared between PB1 and cr-PB1 using a t test with Holm-Sidak comparison. *=p<0.05, ***=p<0.001

### Example 33: Plasmid Based Killing of E. coli and P. Aeruginosa by Type I CRISPR-Cas Systems

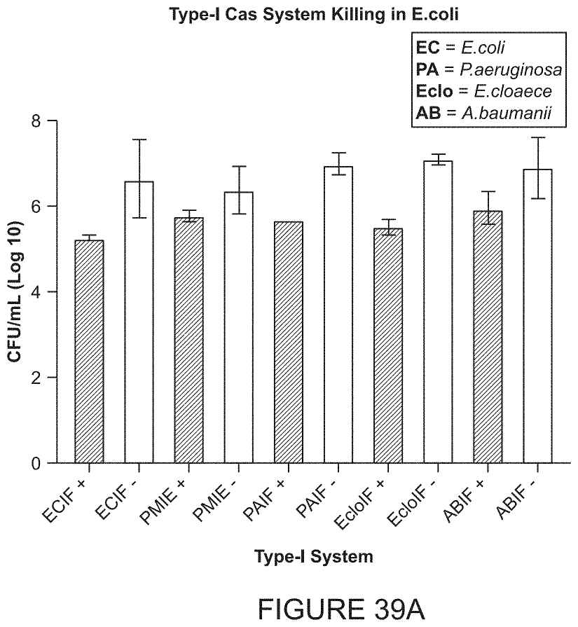

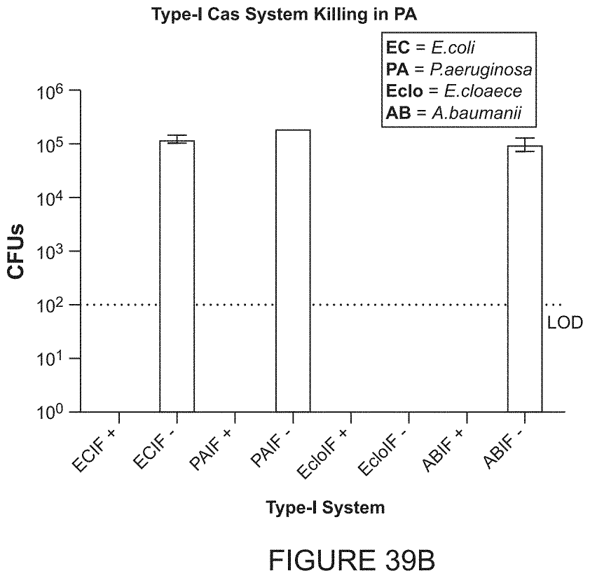

E. coli Type I testing: Five Type-I Cas systems were cloned from bacteria gDNA into pUCP19. Corresponding E. coli (BL21) targeting spacers were cloned into a second compatible plasmid (pRSF1b). B121 electrocompetent cells were transformed with each Cas system plasmid and targeting spacer or pRSF1b control. The transformations were diluted and spot plated on Kan/Carb LB plates. CFUs were counted after overnight incubation at 37 C. Results are exemplified in FIG. 39A.

P. aeruginosa Type-I Testing: Four Type-I Cas systems were cloned from bacteria gDNA into pUCP19 along with a pseudomonas targeting spacer. Electrocompetent PA01 cells were made competent using the Locus lab protocol. The cells were transformed with the Cas+spacer plasmid or Cas plasmid. The transformations were diluted and spot plated on Carb300 LB plates. CFUs were counted after overnight incubation at 37 C. Results are exemplified in FIG. 39B. Results exemplify that 3 of 4 systems were able to successfully target PA01 with a minimum 3-log reduction. 1 system showed no CFUs for either plasmid.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein are employed in practicing the disclosure. It is intended that the following claims define the scope of the disclosure and that methods and structures within the scope of these claims and their equivalents be covered thereby.

What is claimed is:

1. A recombinant obligately lytic bacteriophage having an operable lytic cycle, the recombinant obligately lytic bacteriophage comprising:
(a) a first nucleic acid sequence comprising the genome of the recombinant obligately lytic bacteriophage;
(b) a second nucleic acid sequence encoding a spacer sequence or a crRNA transcribed therefrom, wherein the spacer sequence is complementary to a target nucleotide sequence in a target bacterium; and
(c) a third nucleic acid sequence encoding a Type I Cas polypeptide;
wherein the genome of the recombinant obligately lytic bacteriophage comprises the second and third nucleic acid sequences, and
wherein the recombinant obligately lytic bacteriophage is able to replicate and lyse the target bacterium during a lytic cycle of the recombinant obligately lytic bacteriophage; and the target bacterium is a Pseudomonas bacterium.

2. The recombinant obligately lytic bacteriophage of claim 1, wherein the Pseudomonas bacterium is Pseudomonas aeruginosa.

3. The recombinant obligately lytic bacteriophage of claim 1, wherein the Pseudomonas bacterium is resistant to at least one antibiotic.

4. The recombinant obligately lytic bacteriophage of claim 3, wherein the at least one antibiotic comprises a cephalosporin, fluoroquinolone, carbapenem, colistin, aminoglycoside, vancomycin, streptomycin, or methicillin, or two or more thereof.

5. The recombinant obligately lytic bacteriophage of claim 1, wherein the Type I Cas polypeptide is exogenous to the target bacterium.

6. The recombinant obligately lytic bacteriophage of claim 1, wherein the Type I Cas polypeptide is Cas3.

7. The recombinant obligately lytic bacteriophage of claim 1, further comprising a fourth nucleic acid sequence encoding a Cascade complex.

8. The recombinant obligately lytic bacteriophage of claim 7, wherein the genome of the recombinant obligately lytic bacteriophage comprises the fourth nucleic acid sequence.

9. The recombinant obligately lytic bacteriophage of claim 7, wherein the Cascade complex is exogenous to the target bacterium.

10. The recombinant obligately lytic bacteriophage of claim 7, wherein the Cascade complex comprises a Pseudomonas Type I Cascade complex.

11. The recombinant obligately lytic bacteriophage of claim 10, wherein the Cascade complex comprises a Pseudomonas aeruginosa Type I Cascade complex.

12. The recombinant obligately lytic bacteriophage of claim 7, wherein the Cascade complex comprises:
(a) a Cas7 polypeptide, a Cas8a1 polypeptide or a Cas8a2 polypeptide, a Cas5 poly-peptide, a Csa5 polypeptide, a Cas6a polypeptide, a Cas3′ polypeptide, and a Cas3″ polypeptide having no nuclease activity (Type I-A CRISPR-Cas system);
(b) a Cas6b polypeptide, a Cas8b polypeptide, a Cas7 polypeptide, and a Cas5 poly-peptide (Type I-B CRISPR-Cas system);
(c) a Cas5d polypeptide, a Cas8c polypeptide, and a Cas7 polypeptide (Type I-C CRISPR-Cas system);
(d) a Cas10d polypeptide, a Csc2 polypeptide, a Csc1 polypeptide, a Cas6d polypeptide (Type I-D CRISPR-Cas system);
(e) a Cse1 polypeptide, a Cse2 polypeptide, a Cas7 polypeptide, a Cas5 polypeptide, and a Cas6e polypeptide (Type I-E CRISPR-Cas system); or
(f) a Csy1 polypeptide, a Csy2 polypeptide, a Csy3 polypeptide, and a Cas6f polypeptide (Type I-F CRISPR-Cas system).

13. The recombinant obligately lytic bacteriophage of claim 7, wherein the Cascade complex comprises a Type I-C Cascade complex.

14. A pharmaceutical composition comprising the recombinant obligately lytic bacteriophage of claim 1, and a pharmaceutically acceptable excipient.
